(19) United States
(12) Patent Application Publication
(10) Pub. No.: US 2016/0075732 A1
(43) Pub. Date: Mar. 17, 2016

(54) RADICAL ORBITAL SWITCHING

(71) Applicant: THE AUSTRALIAN NATIONAL UNIVERSITY, Acton, ACT (AU)

(72) Inventors: Michelle Louise Coote, Gundaroo (AU); Ganna Gryn'ova, Aranda (AU)

(73) Assignee: The Australian National University, Acton, ACT (AU)

(21) Appl. No.: 14/765,967

(22) PCT Filed: Feb. 6, 2014
(86) PCT No.: PCT/AU2014/000085

§ 371 Date: Aug. 5, 2015

(30) Foreign Application Priority Data
Feb. 6, 2013 (AU) 2013900371
Feb. 6, 2013 (AU) 2013900373

(51) Int. Cl. C07H 19/207; C07D 207/46; C07D 211/96; C07D 209/46; C07D 221/12; C07F 9/53; C07D 211/94; C07F 9/59

(52) U.S. Cl. CPC C07H 19/207; C07D 211/94; C07D 207/46; C07F 9/59; C07D 209/46; C07D 221/12; C07F 9/5325; C07D 211/96

(57) ABSTRACT

Described herein are distonic radical anion species of formula (I): RAD-L-NEG; wherein RAD is a group comprising a radical; NEG is a group comprising an anion, which is capable of bonding to a proton or other cation; L is a linker that links NEG to RAD; and the radical of RAD is not π-conjugated to the anion of NEG.

### TECHNICAL FIELD

The present invention relates to orbital switching in radicals; in particular to the application of controlled and reversible switching of the Singly Occupied Molecular Orbital (SOMO) from a high energy level (e.g. the Highest Occupied Molecular Orbital (HOMO)) to a lower energy level by direct physical or chemical means.

### BACKGROUND OF THE INVENTION

It is well known that in open-shell species, e.g. radicals, the molecular orbital (MO) accommodating the unpaired electron (the singly-occupied molecular orbital, or SOMO) is usually energetically the highest-occupied molecular orbital (HOMO).

That is, the electronic configuration of an atom or a molecule usually conforms to the aufbau principle, which literally means a ‘building-up’ principle: ‘a maximum of two electrons are put into orbitals in the order of increasing orbital energy’.

However, there are a limited number of open-shell molecules which do not conform to this ‘normal’ aufbau principle of molecular orbital occupation.

These ‘converted’ species include a radical nitronyl nitroxide (NN.) bonded to a tetrathiafulvalene (TTF) (Sugawara; Chem. Soc. Rev. 40, 3105; 2011).

However, in these compounds the control of the energetic ordering of the molecular orbitals by any simple and direct physical or chemical means is unknown. However, it is possible in those cases to remove one or more electrons from doubly-occupied molecular orbitals with energies higher than that of a SOMO by electrochemical processes, but this alters the number of electrons in the system, rather than reordering the energy levels of the existing electrons in the system, by direct physical or chemical means. Even then, there is no way to change these ‘converted’ (i.e. SOMO≠HOMO) species into the more ‘normal’ aufbau species, and as such the more regular molecular orbital configurations (e.g. SOMO=HOMO) by any simple and direct physical or chemical means.

### SUMMARY OF THE INVENTION

According to a first aspect of the invention, there is provided a structure of Formula (I):

RAD-L-NEG (I)
wherein:
   RAD is a group comprising a radical;
   NEG is a group comprising an anion, which is capable of bonding to a proton or other cation;
   L links NEG and RAD;
   the radical of RAD is not π-conjugated to the anion of NEG; and
wherein the lowest Singly-Occupied Molecular Orbital (SOMO) of RAD is lower in energy than a Doubly-Occupied Molecular Orbital (DOMO) of NEG; and wherein the SOMO of RAD is higher in energy than the DOMOs of NEG when the anion of NEG is bonded to a proton or other cation.

A radical of Formula (I) which has the ‘normal’ SOMO=HOMO arrangement can be switched to the ‘converted’ arrangement configuration (i.e. SOMO≠HOMO), by the simple removal of protons (or cations) from the anion of the NEG group.

The addition of protons (or other cations) to the structure of Formula (I) causes the radical to be ‘activated’ and so to become more reactive (activating the radical for reaction, for example for use in radical-based chemistry). Reversing the above process, i.e. removing protons or other cations stabilises the structure of Formula (I), and if suitably stabilised will have reduced reactivity, and any bond formed between the radical of Formula (I) and another moiety can be more easily broken (e.g. by homolytic cleavage), releasing both the structure of Formula (I) and any moiety it had previously been bonded to; or at least the process and/or conditions of removing the structure of Formula (I) from a bonded moiety can be made more moderate (e.g. by using lower temperatures).

In that way, the structure of Formula (I) can be used in chemical synthesis and/or in physical or chemical analysis or in sensors, by reversibly switching the levels of the molecular orbitals of the Structure of Formula (I), to take advantage of the switchable reactivity of the radical of RAD in the system of the invention.

According to a second aspect of the invention, there is provided a structure of Formula (I):

RAD-L-NEG (I)
wherein:
   RAD is a group comprising a radical;
   NEG is a group comprising an anion, which is capable of bonding to a proton or other cation;
   L links NEG and RAD;
   the radical of RAD is not π-conjugated to the anion of NEG; and
wherein, the lowest Singly-Occupied Molecular Orbital (SOMO) of RAD is lower in energy than in the corresponding structure of Formula (I) when the anion of NEG is bonded to a proton or other cation.

As with the first aspect of the invention, the ability to directly and reversibly switch/tune the energy levels of the orbitals of the radical in the structure of Formula (I) allows access to the control over the chemistry of structure of Formula (I).

According to a third aspect of the invention there is provided a structure of Formula (I):

RAD-L-NEG (I)
wherein:
   RAD is a group comprising a radical;
   NEG comprises a negative point charge, which is capable of being neutralised;
   L links NEG and RAD;
   the radical of RAD is not π-conjugated to the negative point charge of NEG; and
wherein, the lowest Singly-Occupied Molecular Orbital (SOMO) of RAD is lower in energy than in the corresponding structure of Formula (I) when the negative point charge of NEG is neutralized.

As with the previous aspects of the invention, the ability to directly and reversibly switch/tune the energy levels of the orbitals of the radical in the structure of Formula (I) allows user control of the chemistry of structure of Formula (I).

According to the third aspect of the invention, NEG is not a chemical group but rather is a functional physical equivalent, which provides the same function as the anion of NEG. That is, the functional physical equivalent comprises a negative point charge, wherein that negative point charge is capable of being neutralized, and wherein the energy level of the unpaired electron of RAD is lowered when the functional physical equivalent of NEG together with RAD-L forms the (corresponding) structure of Formula (I), and wherein the energy level of the unpaired electron of RAD is the highest energy level when the functional physical equivalent is electrically neutralized.

According to an embodiment of the third aspect of the invention, there is provided a structure of Formula (I), wherein NEG is a surface or structure capable of bearing and/or carrying a negative point charge, and where the negative point charge is capable of being neutralised.

According to an embodiment of the third aspect of the invention, there is provided a structure of Formula (I), wherein the negative point charge is selected from the group comprising an electron, an electric charge, a negative potential, or a negative coulombic charge; and where the negative point charge is capable of being neutralised by the substantial removal, dissipation or inversion of that charge.

According to an embodiment of the third aspect of the invention, there is provided a structure of Formula (I), wherein the surface or structure capable of bearing and/or carrying a negative point charge is an electrode.

According to an embodiment of the third aspect of the invention, there is provided a structure of Formula (I), wherein the surface or structure capable of bearing and/or carrying a negative point charge is metallic or non-metallic.

According to an embodiment of the third aspect of the invention, there is provided a structure of Formula (I), wherein the surface or structure capable of bearing and/or carrying a negative point charge comprises graphene.

According to an embodiment of the third aspect of the invention, there is provided a structure of Formula (I), wherein the negative point charge is a group comprising an anion, which is capable of being neutralised by bonding to a proton or other cation.

Accordingly to an embodiment of the third aspect of the invention, there is provided a radical protecting group having the structure of Formula (I); wherein the radical of RAD is capable of forming a bond to a radical to be protected to give the protected radical; and wherein the bond to the radical to be protected is weakened when the negative point charge of NEG is not neutralised.

Accordingly to an embodiment of the third aspect of the invention, there is provided a structure of Formula (I); wherein the radical of RAD is capable of forming a bond to a radical to be protected to give the protected radical; and wherein the radical to be protected is de-protectable when the negative point charge of NEG is not neutralised.

Accordingly to an embodiment of the third aspect of the invention, there is provided a use of the radical protecting group as defined previously to protect a radical, wherein the negative point charge is neutralised.

Accordingly to an embodiment of the third aspect of the invention, there is provided a process of deprotecting a radical protected with a radical protecting group as defined previously, by the removal of the cause of neutralisation from a negative point charge that has been neutralised.

Accordingly to an embodiment of the third aspect of the invention, there is provided a process of deprotecting a radical protected with the radical protecting group as defined previously, comprising the removal the negative point charge; allowing the negative point charge to dissipate; or inverting the negative point charge to a positive point charge.

A positive point charge could for example be used to have substantially the opposite effect as a negative point charge, e.g. to destabilise the radical of RAD, and/or to deprotect a radical protected by a structure of Formula (I).

Accordingly to an embodiment of the third aspect of the invention, there is provided a process of protecting a radical by reacting that radical with the radical protecting group as defined previously, wherein the negative point charge is neutralised.

Accordingly to an embodiment of the third aspect of the invention, there is provided a process of activating the radical protecting group as defined above, by neutralising the negative point charge.

When the negative point charge of NEG is neutralized the lowest Singly-Occupied Molecular Orbital (SOMO) of RAD is higher in energy than in the corresponding structure of Formula (I) when the negative point charge of NEG is not neutralized.

The present invention will be detailed hereafter primarily in respect of the first, second and fourth aspects of the invention. However, it should be understood that (where chemically and physically reasonable) references to the following aspects and embodiments where NEG comprises an anion, also includes within its meaning a NEG that comprises a negative point charge, where this takes the form of a physical functional equivalent (as detailed hereinabove in the fourth aspect of the invention). Likewise, in those situations, references to the anion of NEG bonding to a proton or other cation encompasses within its meaning neutralisation of the negative point charge (as detailed herein above). Likewise, the removal of a proton or other cation from an anion of NEG encompasses within its meaning the removal of the cause of neutralisation (i.e. de-neutralisation), thereby giving/regenerating the negative point charge.

According to a fourth aspect of the invention, there is provided a radical protecting group having the structure of Formula (I):

RAD-L-NEG (I)
wherein:
   RAD is a group comprising a radical;
   NEG is a group comprising an anion, which is capable of bonding to a proton or other cation;
   L links NEG and RAD;
   the radical of RAD is not π-conjugated to the anion of NEG; and
wherein the lowest Singly-Occupied Molecular Orbital (SOMO) of RAD is lower in energy than a Doubly-Occupied Molecular Orbital (DOMO) of NEG; and wherein the SOMO of RAD is higher in energy than the DOMOs of NEG when the anion of NEG is bonded to a proton or other cation; and
wherein the radical of RAD is capable of forming a bond to a radical to be protected when the anion of NEG is bonded to a proton or other cation; and
wherein the radical to be protected is de-protectable when the anion of NEG is not bonded to a proton or other cation.

A radical of Formula (I) which has the ‘normal’ SOMO=HOMO arrangement and where that radical is bound to a radical to be protected, could be released from that radical to be protected by causing the ‘normal’ SOMO=HOMO configuration to be switched to the ‘converted’ arrangement configuration (i.e. SOMO≠HOMO), by the simple removal of protons (or cations) from the anion of the NEG group.

The addition of protons (or other cations) to the structure of Formula (I) causes the radical to be ‘activated’ and so to become more reactive and so that it can form a bond to the radical to be protected. Reversing the above process, i.e. removing protons or other cations stabilises the structure of Formula (I), and if suitably stabilised, the bond formed between the radical to be protected and the structure of Formula (I) breaks (i.e. by homolytic cleavage), releasing both the radical to be protected and the structure of Formula (I); or at least the process and/or conditions of removing the structure of Formula (I) from the structure to be protected can be made more moderate (e.g. using lower temperatures).

When the anion of NEG is bonded to a proton or other cation the lowest Singly-Occupied Molecular Orbital (SOMO) of RAD is higher in energy than in the corresponding structure when the anion of NEG is not bonded to a proton or other cation.

In that way, the structure of Formula (I) can be used as a (reversible) protecting group in chemical synthesis and/or analysis, as indicated in this aspect of the invention.

For example, a portion of a structure can be protected with the structure of Formula (I) e.g. R-RAD-L-NEG (where the ‘R-RAD’ bond comprises the radical electron of RAD and the radical election of R), while various other reactions are conducted on the unprotected portions of that structure. When required, the structure of Formula (I) can be selectively released (e.g. by increasing the pH) from that structure, allowing that portion which had been protected to be unprotected e.g. to give R. and RAD-L-NEG (where NEG is not bonded to a proton or other cation).

The radical to be protected, once deprotected, is free to participate in further reactions. The skilled person will be aware of those reactions, typically being radical type reactions.

In view of the tunability and adaptability of the structure of Formula (I), it is possible for example that more than one protecting group of the invention could be used on the same structure to be protected, whereby each protecting group is deprotected under different reaction conditions, for example each responsive in a certain pH range or in the presence of different cations, or to progressively harsher deprotecting conditions such as increasing temperature. It is also possible that more than one protecting group can be removed (from a structure to be protected) under the same conditions. When two radical protecting groups are removed, the two resulting radicals on the same structure can be arranged such that they can recombine. The two resultant radicals could be arranged such that the cyclization of the structure bearing these radicals occurs when they are deprotected (e.g. an intramolecular Wurtz reaction).

According to an embodiment of the invention, there is provided a use of the radical protecting group as defined previously to protect a radical, wherein the anion of NEG is bonded to a proton or other cation.

According to an embodiment of the invention, there is provided a process of deprotecting a radical protected with a radical protecting group as defined previously, by the removal of the proton or other cation bonded to the anion of NEG.

According to an embodiment of the invention, there is provided a process of deprotecting a radical protected with the radical protecting group as defined previously, by increasing the pH of the reaction medium to remove the proton bonded to the anion of NEG.

According to an embodiment of the invention, there is provided a process of deprotecting a radical protected with the radical protecting group as defined previously, by the addition of a metal chelate to the reaction medium to remove metal cation bonded to the anion of NEG.

According to an embodiment of the invention, there is provided a process of deprotecting a radical protected with the radical protecting group as defined previously, by the addition of anions to the reaction medium, the added anions forming a precipitate with cations present in the reaction medium, thereby removing those cations from bonding with the anions of NEG.

According to an embodiment of the invention, there is provided a process of protecting a radical by reacting that radical with the radical protecting group as defined previously, wherein the anion of NEG is bonded to a proton or other cation.

According to an embodiment of the invention, there is provided a process of activating the radical protecting group as defined previously, by the addition of protons or other cations to the reaction medium, wherein the anion of NEG bonds to added protons or other cations.

According to an embodiment of the invention, there is provided a process of activating the radical protecting group as defined previously, by decreasing the pH of the reaction medium, to protonate the anion of NEG.

According to an embodiment of the invention, there is provided a process of deprotecting a radical protected with the radical protecting group as defined previously, by increasing the polarity of the reaction medium, such that the protons or other cations bonded to the anion of NEG dissociate from that anion.

According to an embodiment of the invention, there is provided a process of activating the radical protecting group as defined previously, by decreasing the polarity of the reaction medium, such that protons or other cations in the reaction medium are bonded to the anion of NEG.

The strong stabilizing effects of charges on radical stability, as demonstrated using accurate quantum chemistry calculations, has been validated experimentally in the gas phase using mass spectrometry, as detailed herein. The pH switch can reveal itself as a reduction in bond dissociation energy (and hence increased tendency of R—X—COO⁻ to dissociate into R.+.X—COO⁻ versus dissociation corresponding protonated compound R—X—COOH into R.+.X—COOH). Or, completely equivalently, it can reveal itself as an increased acidity of the remote carboxylic acid group in the radical form .X COOH versus the parent non-radical R—X—COOH (i.e., R—X—COOH→R—X—COO⁻+H⁺ is less favoured than .X—COOH→.X—COO⁻+H⁺). Both manifestations of this pH switch have been exploited in the experimental verification in solution, in addition to using first principles quantum chemical calculations to calculate directly the effects of solvent on the switch. The results from each of these approaches are detailed with reference to FIG. 15, FIG. 16, FIG. 17, FIG. 18 and with reference to Table 21

According to an embodiment of the invention, there is provided a process wherein one or more of the processes as defined previously are used.

According to an embodiment of the invention, there is provided a process as defined previously wherein the deprotected radical species reacts with itself, or with one or more reagents in the reaction medium.

According to an embodiment of the invention, there is provided a process as defined previously, wherein the radical reaction is selected from the group comprising: radical coupling; Wurtz reaction; nitroxide mediated polymerization; nitroxide radical coupling; double bond addition; cyclization reactions; atom abstraction; and oxidation.

According to an embodiment of the invention, there is provided a process as defined previously wherein the deprotected radical participates in a polymerization reaction.

According to an embodiment of the invention, there is provided a process as defined previously, wherein the chain extending polymer radical is capped with the radical protecting group as defined previously.

According to an embodiment of the invention, there is provided a process as defined previously, wherein the capped radical is deprotected, allowing further polymerization of that deprotected radical.

According to an embodiment of the invention, there is provided a process as defined previously wherein the polymer substrate prior to capping is different from the polymer substrate after deprotection.

According to an embodiment of the invention, there is provided a process wherein one or more of the processes as defined previously are used.

According to an embodiment of the invention, there is provided a process as defined previously wherein the polymerization reaction is a block co-polymerization.

According to an embodiment of the invention, there is provided a process as defined previously wherein the polymerization is conducted in the temperature range of up to 120° C. Preferably, in the temperature range 25 to 80° C.

According to an embodiment of the invention, there is provided a process as defined previously, wherein the reaction medium is buffered.

According to an embodiment of the invention, there is provided a process as defined previously, wherein the cation is a metal ion or metal containing ionic species or is an ammonium or phosphonium ion.

According to an embodiment of the invention, there is provided a process as defined previously, wherein the protected radical, the radical to be protected, or the radical protecting group as defined previously are detectable by a sensor.

According to an embodiment of the invention, there is provided a process as defined previously, wherein the sensor comprises a profluorescent probe.

According to the above embodiment, there is provided a process wherein the profluorescent probe comprises a nitroxide and a fluorophore (i.e. a fluorescent group), wherein the RAD group of the structure of Formula (I) comprises the nitroxide, and wherein the nitroxide radical quenches the fluorescence of the fluorophore. The skilled person is aware of suitable fluorophore groups. When the nitroxide is engaged in a bond and thus the profluorescent probe is in a closed-shell state, the fluorescence is not quenched. The fluorophore of the profluorescent probe can be part of the RAD or NEG group and/or part of L of the structure of Formula (I).

According to an embodiment of the invention, there is provided a process as defined previously, wherein the sensor is useful in determining pH; medical imaging; degree of oxidation or reduction; detecting and quantifying free radical species that may be present.

According to an embodiment of the invention, there is provided a process as defined previously, wherein the sensor is useful in determining pH. For example, when the sensor is a nitroxide profluorescent probe and when the anion of NEG is protonated (or bonded to another cation) the radical of RAD will be destabilised and will react with a suitable in situ species, such that the radical of RAD bonds to that species. In doing so the fluorescence of the corresponding fluorophore group is no longer quenched and fluorescence is therefore detectable. Calibration of the fluorescent response to change in pH (or other cation concentration) gives a sensor to measure pH (or other cation concentration). The opposite process is also possible wherein the removal of protons (or other cations) leads to removal of these from bonding to the anion of NEG, this stabilises the radical of RAD causing its release through a homolytic cleavage of its bond with R in R-RAD-L-NEG and quenching of the fluorophore group.

According to an embodiment of the invention, there is provided a process of monitoring the pH of a medium by measuring the concentration of one or more of the protected radical, the radical to be protected, or the radical protecting group as defined previously; or one or more products resulting from those species.

According to an embodiment of the invention, there is provided a process as defined previously, wherein the sensor is useful in medical imaging; measuring degree of oxidation or reduction; detecting and quantifying free radical species that may be present; detecting anions, wherein the sensor is turned on by the addition of protons (or other cations), or turned off by the removal of protons (or other cations), and wherein this process can be reversible.

According to an embodiment of the invention, there is provided a process as defined previously, wherein the sensor is useful in detecting oxidative stress in a cell.

According to an embodiment of the invention, there is provided a process as defined previously, wherein the sensor is useful in detecting oxidative stress in non-biological systems, such as in machinery (e.g. aircraft parts), or similar object subjected to oxidative stress.

According to an embodiment of the invention, there is provided a process as defined previously, wherein one or more of the above defined profluorescent probes are used to detect oxidative stress in polymers. For example, when a polymer starts to degrade via radical mechanisms, a profluorescent probe (in radical form) can trap the forming radical species, and the fluorescence will occur. The radical form of the profluorescent probe can be released in response to a change in pH.

According to an embodiment of the invention, there is provided a paint incorporating one or more of the above defined profluorescent probes, or a polymer as defined above which comprises the profluorescent probes.

According to an embodiment of the invention, there is provided an anion sensor which results from a structure of Formula (I) forming, where the anion to be detected constitutes the anion of NEG, L is a through space interaction from NEG to RAD. The anion sensor may for example comprise a profluorescent probe which could be located on the RAD group. In this example, as the anion concentration (of the anion to be detected) increases, statistically the formation of the structure of Formula (I) will also increase (that is RAD and NEG are more likely to become close enough to form a structure of Formula (I)). When a structure of Formula (I) is formed the radical of RAD is stabilised by the proximate anion. This can result in the radical of RAD disassociating from a protected group. When the radical of RAD is disassociated from the protected group (to give a non-bonded radical), the profluorescene of the fluorophore group on RAD would be quenched. Suitable calibration would be needed to index the fluorescence to the concentration of anions to be detected. A related process could also be used wherein suitable anions are trapped within a semipermeable membrane together with a RAD group (e.g. which has a fluorophore group) to form a probe; and where the concentration of those anions within the semipermeable membrane is altered by osmotic processes and in so doing the profluorescent response of the probe is likewise changed (in the manner explained above). When such a profluorescent probe is at equilibrium with the medium being measured there will be no further change in the fluorescence of the probe. Again, a simple calibration would be needed to determine the response of the probe under the conditions to be measured. The skilled person would be aware of how such a calibration could be done.

According to an embodiment of the invention, there is provided a process as defined previously, wherein the radical to be protected, or the RAD-L-NEG group, is an antioxidant.

According to an embodiment of the invention, there is provided a process as defined previously, wherein the radical to be protected, or the RAD-L-NEG group, is an industrial antioxidant, inclusive of light stabilisers.

According to an embodiment of the invention, there is provided a process as defined previously, wherein the radical to be protected is a biologically active antioxidant.

According to an embodiment of the invention, there is provided a use of the radical to be protected and/or the radical protecting group as defined previously, as a radical scavenger.

According to an embodiment of the invention, there is provided a process as defined previously, wherein one or more of the radical to be protected or the radical protecting group; a resultant metabolite thereof; or a resultant product thereof is biologically active.

According to an embodiment of the invention, there is provided a process as defined previously wherein biologically active species is released in the body.

According to an embodiment of the invention, there is provided a process as defined previously, wherein biologically active species is released in the body in response to a change in physiological conditions, and in particular as the result of increase in pH.

According to an embodiment of the invention, there is provided a process as defined previously,

wherein the biologically active species is selected from the group:
nitric oxide;
nitrogen dioxide;
a structure comprising a nitric oxide and/or nitrogen dioxide radical; or non-steroidal anti-inflammatory drug (NSAID).

According to an embodiment of the invention, there is provided a process of oxidation of a structure of Formula (I) as defined previously, by the removal of an electron to give a biradical species.

According to an embodiment of the invention, there is provided a process of oxidation of Formula (I) as defined previously, wherein the negative point charge has been neutralised, and wherein oxidation removes an unpaired electron.

According to an embodiment of the invention, there is provided a process of oxidation of a structure of Formula (I) as defined previously, to remove an electron to give a biradical species, wherein the reaction is conducted in vacuum, gas phase, a low polarity solvent or in the solid state.

According to an embodiment of the invention, there is provided a process of oxidation of Formula (I), as defined previously, wherein NEG is bonded to a proton or other cation, and wherein oxidation removes an unpaired electron.

According to an embodiment of the invention, there is provided a process wherein one or more of the processes as defined previously are used.

According to an embodiment of the invention, there is provided a process as defined previously, wherein the species to be oxidised in claim 53 and the species to be oxidized in claim 54 are interconvertible by the introduction or removal of a negative point charge.

According to an embodiment of the invention, there is provided a process as defined previously, wherein the species to be oxidised to a biradical species as defined previously and the species to be oxidised to remove an unpaired electron as defined previously are interconvertible by the addition or removal of protons or other cations.

According to an embodiment of the invention, there is provided a process as defined previously, for use in molecular electronic applications, inclusive of solar cells.

According to a fifth aspect of the invention, there is provided a use of computational means to identify a structure of Formula (I):

RAD-L-NEG (I)

wherein RAD, NEG and L are defined in accordance with anyone of the previous aspects of the invention.

This aspect of the invention allows for the screening of candidate structures by various methods of calculations inclusive of single-reference Hartree-Fock (HF), Density Functional Theory (DFT) and post-HF ab initio (e.g., MP2, CC) methods, including their combinations (high-level composite methods of G3 and G4 families) and multi-reference methods (e.g. MCSCF, MRPT2, CASSCF). These various methods would be readily appreciated by the skilled person.

The use of calculations in this aspect of the invention allows for structures demonstrating the properties of the orbital switching of the invention to be identified and/or designed. Such calculations could include a determination of the stabilisation of the radical in the SOMO≠HOMO state and the acidity of the anion of NEG.

The skilled person is aware of the various ways in which radical stability can be assessed. For example, this may be measured in terms of bond dissociation energy of RAD-R where R is a leaving group R., such as methyl (.CH₃).

Calculations can be performed on a single molecule, part of a molecule, complex or could be done on a composite structure incorporating one or more elements which when taken together make up the structure of Formula (I).

According to an embodiment of the invention, there is provided the resultant structure according to the method as defined previously.

According to a sixth aspect of the invention, there is provided a method of stabilising a radical bearing structure, comprising the introduction of negative point charge to that structure, wherein the resultant structure formed is Formula (I):

RAD-L-NEG (I)

wherein RAD, NEG and L are defined in accordance with the third aspects of the invention.

According to a seventh aspect of the invention, there is provided a method of stabilising a radical bearing structure, comprising the incorporation of an anion into that structure, wherein the resultant structure formed is Formula (I):

RAD-L-NEG (I)
wherein:
   RAD is a group comprising the radical;
   NEG is a group comprising the anion, which is capable of bonding to a proton or other cation;
   L links NEG and RAD;
   the radical of RAD is not π-conjugated to the anion of NEG; and
      wherein RAD, NEG and L are defined in accordance with anyone of the previous aspects of the invention.

According to this aspect of the invention there is provided a method in which an anion is incorporated into a structure such that the structure formed is of Formula (I).

For example an existing structure could be modified to include an anion in an arrangement such that an orbital switching structure of the invention is obtained. This might be for example used to make a superior agent for use in radical polymerization reactions, such as in nitroxide mediated polymerization. Such reagent could be designed to have lower activation thresholds.

According to an embodiment of the invention, there is provided the resultant structure according to the method as defined previously.

According to an eighth aspect of the invention, there is provided a method of decreasing the pKₐ of a conjugate acid bearing structure, comprising the incorporation of a radical into that structure, wherein the resultant structure is Formula (I):

RAD-L-NEG (I)

wherein RAD, NEG and L are defined in accordance with anyone of the previous aspects of the invention.

According to this aspect of the invention there is provided a method in which a radical is incorporated into a structure such that the structure formed is of Formula (I).

For example a known structure could be modified to include a radical in an arrangement such that an orbital switching structure of the invention is obtained. This might be for example used to make an acid group present in the structure more acidic, or to make superior agent for use in radical polymerization reactions, such as in nitroxide mediated polymerization. Such reagent could be designed to have lower activation thresholds.

According to an embodiment of the invention, there is provided the resultant structure according to the method as defined previously.

According to a ninth aspect of the invention, there is provided a method of lowering the energy level of the Singly-Occupied Molecular Orbital of RAD, by the removal of the cause of neutralisation from a point charge of NEG that has been neutralised, in a structure of Formula (I):

RAD-L-NEG (I)

wherein RAD, NEG and L are defined in accordance with the third aspects of the invention.

According to a tenth aspect of the invention, there is provided a method of raising the energy level of a Doubly-Occupied Molecular Orbitals of NEG above the energy level of the Singly-Occupied Molecular Orbital of RAD, by the removal of protons and/or other cations from the anion of NEG, in a structure of Formula (I):

RAD-L-NEG (I)

wherein RAD, NEG and L are defined in accordance with anyone of the previous aspects of the invention.

The use of this method could be employed were it is desirable to switch the orbitals from the ‘converted’ electronic configuration to the ‘normal’ aufbau-type configuration. This may for example be used to activate a structure of Formula (I) to a more reactive state. For example, where the stabilised radical of Formula (I) is in solution but substantively inactive under the reaction conditions, treatment with proton acid could protonate the anion of NEG causing the orbitals to revert to the more normal aufbau electronic configuration. The radical is thus destabilised, and is so more reactive, and for example could be used to react with other radicals in solution or to cap or protect a reactive group.

Equally, it is contemplated that the reverse of this process is within the scope of the invention, wherein the protons and/or other cations are removed from the anion of NEG of the structure of Formula (I), with the purpose of switching the orbitals from the ‘normal’ aufbau-type configuration to the ‘converted’ configuration, and as such, this reverse process is considered to be within the scope of the above aspect of the invention. In that way for example, a group capped or protected with the structure of Formula (I) can be deprotected.

According to an embodiment of the invention, there is provided the resultant structure according to the method as defined previously.

According to a eleventh aspect of the invention, there is provided a method of modifying a biological macromolecule comprising the incorporation of a radical into that biological macromolecule, wherein the resultant structure formed is Formula (I):

RAD-L-NEG (I)
wherein:
   RAD is a group comprising the radical;
   NEG is a group comprising an anion, which is capable of bonding to a proton or other cation;
   L links NEG and RAD;
   the radical of RAD is not π-conjugated to the anion of NEG; and
      wherein after the modification, the lowest Singly-Occupied Molecular Orbital (SOMO) of RAD is lower in energy than in the corresponding structure of Formula (I) when the anion of NEG is bonded to a proton or other cation.

According to a twelfth aspect of the invention, there is provided a method of modifying a biological macromolecule comprising the incorporation of a radical into that biological macromolecule, wherein the resultant structure formed is Formula (I):

RAD-L-NEG (I)
wherein:
   RAD is a group comprising the radical;
   NEG is a group comprising an anion, which is capable of bonding to a proton or other cation;
   L links NEG and RAD;
   the radical of RAD is not π-conjugated to the anion of NEG; and
      wherein after the modification, the lowest Singly-Occupied Molecular Orbital (SOMO) of RAD is lower in energy than a Doubly-Occupied Molecular Orbital (DOMO) of NEG; and wherein the SOMO of RAD is higher in energy than the DOMOs of NEG when the anion of NEG is bonded to a proton or other cation.

According to this aspect of the invention there is provided the modification of a biological macromolecule (such as an enzyme) to provide a structure of Formula (I). In this method the biological macromolecule could be structurally changed by chemical reaction (addition, substitution, elimination, removal, rearrangement or some other modification) such that the group to be added is added to the biological macromolecule. Also for example, a sub-unit of the biological unit could be swapped for a new sub-unit bearing the required group. The skilled person will be aware of ways in which this could be done. Biologically driven transformations are also envisioned, in which the modification is brought about for example by an enzyme. Again, the skilled person will be aware of ways in which this could be done. Considered within the meaning of the radical of RAD is a radical precursor, such that the radical precursor is added to the biological macromolecule, and wherein the radical of RAD is released in situ (in the body in situ would be understood to be in vivo). For example an Activating Enzyme might release the radical from the radical precursor in situ.
According to a thirteenth aspect of the invention, there is provided a biological macromolecule comprising the incorporation of an anion into a biological macromolecule, wherein the resultant structure formed is Formula (I):

RAD-L-NEG (I)
wherein:
   RAD is a group comprising a radical;
   NEG is a group comprising the anion, which is capable of bonding to a proton or other cation;
   L links NEG and RAD;
   the radical of RAD is not π-conjugated to the anion of NEG; and
      wherein after the modification, the lowest Singly-Occupied Molecular Orbital (SOMO) of RAD is lower in energy than in the corresponding structure of Formula (I) when the anion of NEG is bonded to a proton or other cation.

According to a fourteenth aspect of the invention, there is provided a method of modifying a biological macromolecule comprising the incorporation of an anion into a biological macromolecule, wherein the resultant structure formed is Formula (I):

RAD-L-NEG (I)
wherein:
   RAD is a group comprising a radical;
   NEG is a group comprising the anion, which is capable of bonding to a proton or other cation;
   L links NEG and RAD;
   the radical of RAD is not π-conjugated to the anion of NEG; and
      wherein after the modification, the lowest Singly-Occupied Molecular Orbital (SOMO) of RAD is lower in energy than a Doubly-Occupied Molecular Orbital (DOMO) of NEG; and wherein the SOMO of RAD is higher in energy than the DOMOs of NEG when the anion of NEG is bonded to a proton or other cation.

According to this aspect of the invention there is provided the modification of a biological macromolecule (such as an enzyme) to provide a structure of Formula (I). In this method the biological macromolecule could be structurally changed by chemical reaction (addition, substitution, elimination, removal, rearrangement or some other modification) such that the group to be added is added to the biological macromolecule. Also for example, a sub-unit of the biological unit could be swap for a new sub-unit bearing the required group. The skilled person will be aware of ways in which this could be done. Biologically driven transformations are also envisioned, in which the modification is brought about for example by an enzyme. Again, the skilled person will be aware of ways in which this could be done.

According to a fifteen aspect of the invention, there is provided a method of modifying a biological macromolecule comprising the incorporation of a radical and an anion into that biological macromolecule, wherein the resultant structure formed is Formula (I):

RAD-L-NEG (I)
wherein:
   RAD is a group comprising the radical;
   NEG is a group comprising the anion, which is capable of bonding to a proton or other cation;
   L links NEG and RAD;
   the radical of RAD is not π-conjugated to the anion of NEG; and
      wherein after the modification, the lowest Singly-Occupied Molecular Orbital (SOMO) of RAD is lower in energy than in the corresponding structure of Formula (I) when the anion of NEG is bonded to a proton or other cation.

According to an sixteenth aspect of the invention, there is provided a method of modifying a biological macromolecule comprising the incorporation of a radical and an anion into that biological macromolecule, wherein the resultant structure formed is Formula (I):

RAD-L-NEG (I)
wherein:
   RAD is a group comprising the radical;
   NEG is a group comprising the anion, which is capable of bonding to a proton or other cation;
   L links NEG and RAD;
   the radical of RAD is not π-conjugated to the anion of NEG; and
      wherein after the modification, the lowest Singly-Occupied Molecular Orbital (SOMO) of RAD is lower in energy than a Doubly-Occupied Molecular Orbital (DOMO) of NEG; and wherein the SOMO of RAD is higher in energy than the DOMOs of NEG when the anion of NEG is bonded to a proton or other cation.

According to this aspect of the invention there is provided the modification of a biological macromolecule (such as an enzyme) to provide a structure of Formula (I). In this method the biological macromolecule could be structurally changed by chemical reaction (addition, substitution, elimination, removal, rearrangement or some other modification) such that the groups to be added are added to the biological macromolecule. Also for example, a sub-unit of the biological unit could be swap for a new sub-unit bearing the required group. The skilled person will be aware of ways in which this could be done. Biologically driven transformations are also envisioned, in which the modification is brought about for example by an enzyme. Again, the skilled person will be aware of ways in which this could be done.
According to a seventeenth aspect of the invention, there is provided a method of modifying a biological macromolecule, wherein the resultant structure formed is Formula (I):

RAD-L-NEG (I)
wherein:
   RAD is a group comprising a radical;
   NEG is a group comprising an anion, which is capable of bonding to a proton or other cation;
   L links NEG and RAD;
   the radical of RAD is not π-conjugated to the anion of NEG; and
      wherein after the modification, the lowest Singly-Occupied Molecular Orbital (SOMO) of RAD is lower in energy than in the corresponding structure of Formula (I) when the anion of NEG is bonded to a proton or other cation.

According to a eighteenth aspect of the invention, there is provided a method of modifying a biological macromolecule, wherein the resultant structure formed is Formula (I):

RAD-L-NEG (I)
wherein:
   RAD is a group comprising a radical;
   NEG is a group comprising an anion, which is capable of bonding to a proton or other cation;
   L links NEG and RAD;
   the radical of RAD is not π-conjugated to the anion of NEG; and
      wherein after modification, the lowest Singly-Occupied Molecular Orbital (SOMO) of RAD is lower in energy than a Doubly-Occupied Molecular Orbital (DOMO) of NEG; and wherein the SOMO of RAD is higher in energy than the DOMOs of NEG when the anion of NEG is bonded to a proton or other cation.

According to this aspect of the invention there is provided the modification of a biological macromolecule (such as an enzyme) to provide a structure of Formula (I). In this method the biological macromolecule could be structurally changed by chemical reaction (addition, substitution, elimination, removal, rearrangement or some other modification) such that the existing structure of the biological macromolecule is modified. For example a new bond could be formed within the structure of the biological macromolecule such that two formerly distant parts of the biological macromolecule are brought closer together (or to tether an Activating Enzyme (or fragment thereof) to part of the enzyme to be activated) to give a structure of Formula (I). In the same way a part or sub-unit of the biological macromolecule could be removed or substituted such that two formerly distant parts of the biological macromolecule are brought closer together (i.e. to brought into closer proximity) to give a structure of Formula (I). The skilled person will be aware of ways in which this could be done. These transformations could be chemically or biologically driven. The skilled person will be aware of ways in which this could be done.

According to a nineteenth aspect of the invention, there is provided a method of modifying a biological macromolecule, such that the resultant biological macromolecule will form a complex with a substrate, wherein that resultant substrate-complex formed is Formula (I):

RAD-L-NEG (I)
wherein:
   RAD is a group comprising a radical;
   NEG is a group comprising an anion, which is capable of bonding to a proton or other cation;
   L links NEG and RAD;
   the radical of RAD is not π-conjugated to the anion of NEG; and
      wherein after the modification, the lowest Singly-Occupied Molecular Orbital (SOMO) of RAD is lower in energy than in the corresponding structure of Formula (I) when the anion of NEG is bonded to a proton or other cation.

According to a twentieth aspect of the invention, there is provided a method of modifying a biological macromolecule, such that the resultant biological macromolecule will form a complex with a substrate, wherein that resultant substrate-complex formed is Formula (I):

RAD-L-NEG (I)
wherein:
   RAD is a group comprising a radical;
   NEG is a group comprising an anion, which is capable of bonding to a proton or other cation;
   L links NEG and RAD;
   the radical of RAD is not π-conjugated to the anion of NEG; and
      wherein after modification, the lowest Singly-Occupied Molecular Orbital (SOMO) of RAD is lower in energy than a Doubly-Occupied Molecular Orbital (DOMO) of NEG; and wherein the SOMO of RAD is higher in energy than the DOMOs of NEG when the anion of NEG is bonded to a proton or other cation.

According to this aspect of the invention there is provided the modification of a biological macromolecule (such as an enzyme) such that the resultant biological macromolecule will form a complex with a substrate, wherein that resultant substrate-complex formed is Formula (I). In this way, it is the combination in three dimensional space of the biological macromolecule and the substrate that forms the structure of Formula (I). For example, the biological macromolecule (so modified) may contain one of the RAD/NEG groups and the substrate may contain the other RAD/NEG group. The Linker may be the body of the biological macromolecule or simply one or more hydrogen bonds (or a combination of the biological macromolecule, substrate and hydrogen bonds). It may however, be that it is the substrate that brings the units of RAD and NEG (of the modified biological macromolecule) together in space to form the structure of Formula (I) i.e. when the substrate is bound.

In this method the biological macromolecule could be structurally changed by chemical reaction (addition, substitution, elimination, removal, rearrangement or some other modification) such that the group to be added is added to the biological macromolecule. Also for example, a sub-unit of the biological unit could be swap for a new sub-unit bearing the required group. The skilled person will be aware of ways in which this could be done. Biologically driven transformations are also envisioned, in which the modification is brought about for example by an enzyme. Again, the skilled person will be aware of ways in which this could be done.

According to a twenty first aspect of the invention, there is provided a method of modifying a substrate, such that the resultant modified substrate will form a complex with a biological macromolecule, wherein that resultant substrate-complex formed is Formula (I):

RAD-L-NEG (I)
wherein:
   RAD is a group comprising a radical;
   NEG is a group comprising an anion, which is capable of bonding to a proton or other cation;
   L links NEG and RAD;
   the radical of RAD is not π-conjugated to the anion of NEG; and
      wherein after the modification, the lowest Singly-Occupied Molecular Orbital (SOMO) of RAD is lower in energy than in the corresponding structure of Formula (I) when the anion of NEG is bonded to a proton or other cation.

According to a twenty second aspect of the invention, there is provided a method of modifying a substrate, such that the resultant modified substrate will form a complex with a biological macromolecule, wherein that resultant substrate-complex formed is Formula (I):

RAD-L-NEG (I)
wherein:
   RAD is a group comprising a radical;
   NEG is a group comprising an anion, which is capable of bonding to a proton or other cation;
   L links NEG and RAD;
   the radical of RAD is not π-conjugated to the anion of NEG; and
      wherein after modification, the lowest Singly-Occupied Molecular Orbital (SOMO) of RAD is lower in energy than a Doubly-Occupied Molecular Orbital (DOMO) of NEG; and wherein the SOMO of RAD is higher in energy than the DOMOs of NEG when the anion of NEG is bonded to a proton or other cation.

According to this aspect of the invention there is provided the modification of a substrate (i.e. one that is involved in a biological process) such that a biological macromolecule will form a complex with that modified substrate, wherein that resultant substrate-complex formed is Formula (I). In this way, it is the combination in three dimensional space of the biological macromolecule and the substrate that forms the structure of Formula (I). For example, the substrate (so modified) may contain one of the RAD/NEG groups and the biological macromolecule may contain the other RAD/NEG group. The Linker may be the body of the biological macromolecule or simply one or more hydrogen bonds (or a combination of the biological macromolecule, substrate and hydrogen bonds). It may however, be that it is the modified substrate that brings the units of RAD and NEG together in space to form the structure of Formula (I) i.e. when the substrate is bound.

In this method the substrate could be structurally changed by chemical reaction (addition, substitution, elimination, removal, rearrangement or some other modification) such that the group to be added is added to the substrate. The skilled person will be aware of ways in which this could be done. Biologically driven transformations are also envisioned, in which the modification is brought about for example by an enzyme. Again, the skilled person will be aware of ways in which this could be done.

According to a twenty third aspect of the invention, there is provided a method of modifying a biological macromolecule and a substrate, such that the resultant modified biological macromolecule and substrate will form a complex, wherein that resultant substrate-complex formed is Formula (I):

RAD-L-NEG (I)
wherein:
   RAD is a group comprising a radical;
   NEG is a group comprising an anion, which is capable of bonding to a proton or other cation;
   L links NEG and RAD;
   the radical of RAD is not π-conjugated to the anion of NEG; and
      wherein after the modification, the lowest Singly-Occupied Molecular Orbital (SOMO) of RAD is lower in energy than in the corresponding structure of Formula (I) when the anion of NEG is bonded to a proton or other cation.

According to a twenty fourth aspect of the invention, there is provided a method of modifying a biological macromolecule and a substrate, such that the resultant modified biological macromolecule and substrate will form a complex, wherein that resultant substrate-complex formed is Formula (I):

RAD-L-NEG (I)
wherein:
   RAD is a group comprising a radical;
   NEG is a group comprising an anion, which is capable of bonding to a proton or other cation;
   L links NEG and RAD;
   the radical of RAD is not π-conjugated to the anion of NEG; and
      wherein after modification, the lowest Singly-Occupied Molecular Orbital (SOMO) of RAD is lower in energy than a Doubly-Occupied Molecular Orbital (DOMO) of NEG; and wherein the SOMO of RAD is higher in energy than the DOMOs of NEG when the anion of NEG is bonded to a proton or other cation.

According to this aspect of the invention there is provided the modification of a substrate (i.e. one that is involved in a biological process) and a biological macromolecule such that a biological macromolecule will form a complex with that substrate, wherein that resultant substrate-complex formed is Formula (I). In this way, it is the combination in three dimensional space of the biological macromolecule and the substrate that forms the structure of Formula (I). For example, the substrate (so modified) may contain one of the RAD/NEG groups and the biological macromolecule (so modified) may contain the other RAD/NEG group. The Linker may be the body of the biological macromolecule or simply one or more hydrogen bonds (or a combination of the biological macromolecule, substrate and hydrogen bonds). It may however, be that it is the modified substrate that brings the units of RAD and NEG together in space to form the structure of Formula (I) i.e. when the substrate is bound.

In this method the biological macromolecule and/or substrate could be structurally changed by chemical reaction (addition, substitution, elimination, removal, rearrangement or some other modification) such that the group to be added is added to the biological macromolecule or substrate. Also for example, a sub-unit of the biological unit could be swapped for a new sub-unit bearing the required group. The skilled person will be aware of ways in which this could be done. Biologically driven transformations are also envisioned, in which the modification to the biological macromolecule or substrate is brought about for example by an enzyme. Again, the skilled person will be aware of ways in which this could be done.

According to an embodiment of the invention, there is provided a method of modifying a biological macromolecule as defined previously, wherein that modification is to the active site of that biological macromolecule.

According to an embodiment of the invention, there is provided a method of modifying a biological macromolecule or substrate as defined previously, wherein that modification changes the stability of the radical to alter the reactivity of the modified enzyme. For example the stability of the radical of RAD is changed such that (i) an Activating Enzyme (such as Pyruvate Formate Lyase Activating Enzyme (PFL-AE)) will act on the radical precursor to form the radical of RAD in situ, or such that the radical of RAD is formed more or less readily in situ, or that the Activating Enzyme cannot form the radical of RAD in situ; (ii) the radical of RAD will not participate in one or more further biochemical reactions (i.e. the enzyme is turned off in part, or turned off completely); (iii) the radical of RAD will participate in one or more further biochemical reaction (i.e. the enzyme is not fully turned off). In these examples, the stability of the radical of RAD can be altered by changing the spatial arrangement (e.g. separation) of the anion of NEG to the radical of RAD in three dimensional space. The anion of NEG can be arranged in three dimensional space (e.g. by the placement of this on residues proximate to RAD) such that the stabilising effect on the radical is altered. An optimum or desired through-space distance might first be estimated through calculations. Further calculations could be used to determine which residues could be modified to give the calculated separation. Without being bound by theory, the stabilising through-space effect appears to be dependent on an inverse of the distance between the anion of NEG and the radical of RAD.

According to an embodiment of the invention, there is provided a method of modifying a biological macromolecule or substrate as defined previously, wherein that modification destroys or prevents the formation of a structure of Formula (I). Such a modification could be used to stop the activity of an enzyme which relies on the formation of a structure of Formula (I). For example, this modification could be used to turn off the activity of an enzyme. Such a modification of the biological macromolecule or substrate could be the removal of one or more of the radical of RAD or the anion of NEG, or wherein the said modification prevents the radical of RAD and the anion of NEG from acting through-space together to provide the orbital conversion, i.e. give SOMO≠HOMO configuration as defined previously. For example, in the wild type of Pyruvate Formate Lyase (PFL) the G734 glycine residue could be altered to prevent hydrogen abstraction (by the wild type Pyruvate Formate Lyase Activating Enzyme (PFL-AE)) to give the radical of RAD, or for example any anions on NEG could be changed to be non-anionic. For example, the carboxylic group of the aspartate on D16 on the wild type of PFL-AE could be replaced with a non-anionic group.

PFL can be useful in the catalyses of the condensation of CoA and pyruvate to form acetyl-CoA and formate (e.g. see FIG. 13).

The reversal of the above process (i.e. to add a radical, radical precursor, or anion or anion precursor) could also be conducted, according to the aspects of the invention defined hereinabove, for example to restore the activity of PFL which has been turned off.

According to an embodiment of the invention, there is provided a modified biological macromolecule, and/or modified substrate as defined previously.

According to an embodiment of the invention, there is provided use of a modified biological macromolecule, and/or modified substrate as defined previously.

According to a twenty fifth aspect of the invention, there is provided a method of medical treatment of a medical condition, the method comprising the administration of a therapeutically effective amount of a compound comprising a structure of Formula (I), as defined previously.

According to a twenty sixth aspect of the invention, there is provided a compound comprising a structure of Formula (I), as defined previously, for use in therapy.

According to a twenty seventh aspect of the invention, there is provided a use of a compound comprising a structure of Formula (I), as defined previously, for the manufacture of a medicament for the treatment and/or prevention of a medical condition.

According to an embodiment of the invention, there is provided the resultant structure according to the method as defined previously.

According to a twenty eighth aspect of the invention, there is provided a use of a compound comprising a structure of Formula (I), as defined previously, for the treatment of a medical condition.

According to a twenty ninth aspect of the invention, there is provided a method, use, process, structure, protecting group, metabolite, biological macromolecule or substrate as defined previously, substantially as hereinbefore described with reference to any one of the examples or the description.

According to an embodiment of the invention, there is provided a method, use, process, structure or protecting group as defined previously, wherein the radical of RAD is stabilised.

The skilled person is aware of the various ways in which radical stability can be assessed. For example, this may be measured in terms of bond dissociation energy of RAD-R where R is a leaving group R., such as methyl (.CH₃).

According to an embodiment of the invention, there is provided a method, use, process, structure or protecting group as defined previously, wherein the radical of RAD is delocalised.

According to an embodiment of the invention, there is provided a method, use, process, structure or protecting group as defined previously, wherein the radical of RAD is stabilised by a captodative effect (i.e. simultaneous resonance with a lone-pair donor and a π-acceptor, as for example on the backbone of a protein or a peptide).

According to an embodiment of the invention, there is provided a method, use, process, structure or protecting group as defined previously, wherein the radical of RAD is in conjugation with L.

According to an embodiment of the invention, there is provided a method, use, process, structure or protecting group as defined previously, wherein RAD comprises a steric and/or electronically stabilised radical.

According to an embodiment of the invention, there is provided a method, use, process, structure or protecting group as defined previously, wherein the radical of RAD is electronically and/or sterically stabilised by groups which are proximate to that radical.

According to an embodiment of the invention, there is provided a method, use, process, structure or protecting group as defined previously, wherein RAD comprises an electronically stabilised radical.

According to an embodiment of the invention, there is provided a method, use, process, structure or protecting group as defined previously, wherein RAD comprises a radical selected from the group:

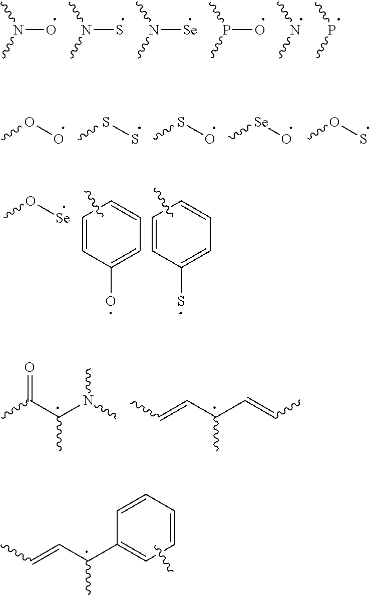

DNA/RNA-base based radical, or is an amino acid based radical.

According to an embodiment of the invention, there is provided a method, use, process, structure or protecting group as defined previously, wherein the DNA/RNA-base is selected from guanine (G) adenine (A), cytosine (C), thymine (T) or uracil (U).

According to an embodiment of the invention, there is provided a method, use, process, structure or protecting group as defined previously, wherein RAD comprises a radical group selected from the group comprising
2,2′,6,6′-tetramethylpiperidine-N-oxyl (TEMPO),
2,2′,5,5′-tetramethylpyrrolidine-N-oxyl (PROXYL),
2,2,5,5-tetramethyl-4-phenyl-3-azahexane-3-oxyl (TIPNO),
N,N-(1,1-dimethylethyl-1)-(1-diethyl-phosphono-2,2-dimethyl-propyl-1)-N-oxyl (SG1), guanine-based or glycyl-based radical.

According to an embodiment of the invention, there is provided a method, use, process, structure or protecting group as defined previously, wherein the anion of NEG is destabilised.

According to an embodiment of the invention, there is provided a method, use, process, structure or protecting group as defined previously, wherein the negative charge of the anion of NEG is not delocalised to any groups outside of that anion.

According to an embodiment of the invention, there is provided a method, use, process, structure or protecting group as defined previously, wherein anion of NEG is not in π-conjugation with L.

According to an embodiment of the invention, there is provided a method, use, process, structure or protecting group as defined previously, wherein NEG comprises a sterically and/or electronically destabilised anion.

According to an embodiment of the invention, there is provided a method, use, process, structure or protecting group as defined previously, wherein anion of NEG is electronically and/or sterically destabilised by groups which are proximate to that anion.

According to an embodiment of the invention, there is provided a method, use, process, structure or protecting group as defined previously, wherein the anion of NEG is destabilised by an electron rich group (e.g. a carbonyl group) which is proximate to the anion of NEG, in particular where that anion is a carboxylate group, for example in a structure comprising the group —CO—CO₂⁻.

According to an embodiment of the invention, there is provided a method, use, process, structure or protecting group as defined previously, wherein NEG comprises an electronically destabilised anion.

According to an embodiment of the invention, there is provided a method, use, process, structure or protecting group as defined previously, wherein NEG comprises an anion selected from the group comprising

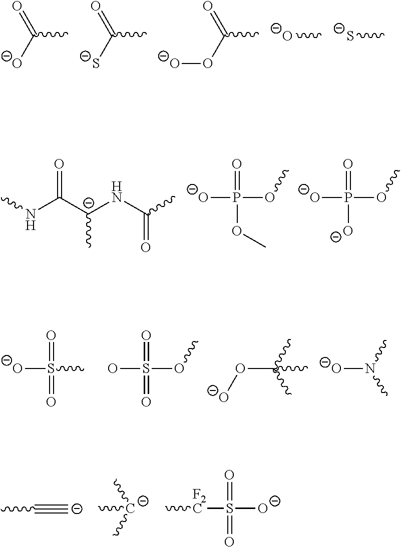

According to an embodiment of the invention, there is provided a method, use, process, structure or protecting group as defined previously, wherein NEG comprises an anion which is a carboxylate group, and wherein that carboxylate group is a constituent part of an amino acid residue. The skilled person is aware of such amino acid residues (e.g. residue based on any one of: alanine, arginine, asparagine, aspartate, cysteine, glutamine, glutamate, glycine, histidine, isoleucine, leucine, lysine, methionine, phenylalanine, proline, serine, threonine, tryptophan, tyrosine and valine; or a modified or unnatural amino acid).

According to an embodiment of the invention, there is provided a method, use, process, structure or protecting group as defined previously, wherein NEG comprises an anion which is a carboxylate group, and wherein that carboxylate group is a constituent part of an amino acid residue, wherein that residue is an aspartate, or the anion of NEG comprises a pyruvate structure.

According to an embodiment of the invention, there is provided a method, use, process, structure or protecting group as defined previously, wherein NEG comprises an anion which is a phosphate, and wherein that phosphate is a mono, di or triphosphate.

According to an embodiment of the invention, there is provided a method, use, process, structure or protecting group as defined previously, wherein L comprises conjugation to RAD.

According to an embodiment of the invention, there is provided a method, use, process, structure or protecting group as defined previously, wherein L does not comprise π-conjugation to NEG.

According to an embodiment of the invention, there is provided a method, use, process, structure or protecting group as defined previously, wherein L is comprised of two or more portions, and wherein at least two of said portions are non-covalently bonded together.

According to an embodiment of the invention, there is provided a method, use, process, structure or protecting group as defined previously, wherein the non-covalent bonding is hydrogen bonding or electrostatic bonding.

According to an embodiment of the invention, there is provided a method, use, process, structure or protecting group as defined previously, wherein the one or more portions comprise one or more DNA/RNA-bases, amino acids, peptides, cofactors, enzymes, enzyme fragments, activating enzymes, biological macromolecules or enzyme substrates, wherein said portions are naturally occurring, modified from those naturally occurring, or are synthetic.

According to an embodiment of the invention, there is provided a method, use, process, structure or protecting group as defined previously, wherein L is comprised of two or more portions, and wherein at least two of said portions bond to one or more metal centres.

According to an embodiment of the invention, there is provided a method, use, process, structure or protecting group as defined previously, wherein the metal centre is selected from the group comprising alkali metal cations and transition metal cations.

Alkali metal cations for example including lithium, sodium and potassium.

Transitional metals for example including those which are mono-, di- or tri-cationic.

According to an embodiment of the invention, there is provided a method, use, process, structure or protecting group as defined previously, wherein L links more than one NEG and/or RAD groups.

According to an embodiment of the invention, there is provided a method, use, process, structure or protecting group as defined previously, wherein L comprises one or more polymeric portions.

According to an embodiment of the invention, there is provided a method, use, process, structure or protecting group as defined previously, wherein L comprises one or more of: a bond, a hydrogen bond; a non-covalent bond; an electrostatic bond; metal bonding; alkyl, cyclic alkyl, aryl, alkene, alkyne, heterocyclic, heteroaromatic, sugar, metal complex, or is a through-space interaction.

According to an embodiment of the invention when L is a through-space interaction, the separation of an atom bearing the anion of NEG and an atom bearing the radical of RAD is sufficient to allow the formation of a structure of Formula (I). That is, at this separation, the lowest Singly-Occupied Molecular Orbital (SOMO) of RAD is lower in energy than a Doubly-Occupied Molecular Orbital (DOMO) of NEG; and wherein the SOMO of RAD is higher in energy than the DOMOs of NEG when the anion of NEG is bonded to a proton or other cation.

According to an embodiment of the invention, there is provided a method, use, process, structure or protecting group as defined previously, wherein L is selected from the group comprising: a bond; phenyl; biphenyl; C₁ to C₂₀ alkyl, C₃ to C₂₀ cycloalkyl, RNA-based sugar; DNA-based sugar.

According to an embodiment of the invention, there is provided a method, use, process, structure or protecting group as defined previously, wherein an atom bearing the negative charge of the anion of NEG and an atom bearing the unpaired electron of the radical of RAD are separated by a through-space distance of about 15 Å or less.

According to an embodiment of the invention, there is provided a method, use, process, structure or protecting group as defined previously, wherein an atom bearing the negative charge of the anion of NEG and an atom bearing the unpaired electron of the radical of RAD are separated by a through-space distance of about 10 Å or less.

According to an embodiment of the invention, there is provided a method, use, process, structure or protecting group as defined previously, wherein an atom bearing the negative charge of the anion of NEG and an atom bearing the unpaired electron of the radical of RAD are separated by a through-space distance of about 5 Å or less.

According to an embodiment of the invention, there is provided a method, use, process, structure or protecting group as defined previously, wherein an atom bearing the negative charge of the anion of NEG and an atom bearing the unpaired electron of the radical of RAD are separated by a through-space distance of about 5 to 15 Å.

According to an embodiment of the invention, there is provided a method, use, process, structure or protecting group as defined previously, wherein an atom bearing the negative charge of the anion of NEG and an atom bearing the unpaired electron of the radical of RAD are separated by a through-space distance of about 5 to 10 Å.

According to an embodiment of the invention, there is provided a method, use, process, structure or protecting group as defined previously, wherein an atom bearing the negative charge of the anion of NEG and an atom bearing the unpaired electron of the radical of RAD are separated by a through-space distance of about 10 to 15 Å.

According to an embodiment of the invention, there is provided a method, use, process, structure or protecting group as defined previously, wherein an atom bearing the negative charge of the anion of NEG and an atom bearing the unpaired electron of the radical of RAD are separated by a through-space distance of about 5 to 7 Å.

According to an embodiment of the invention, there is provided a method, use, process, structure or protecting group as defined previously, wherein the Formula (I) comprises an ionic resin, wherein the resin may comprise negative or positively charged groups.

According to an embodiment of the invention, there is provided a method, use, process, structure or protecting group as defined previously, but wherein the anion of NEG is a neutral functional equivalent, which provides the same function as the anion of NEG. For example, the neutral functional equivalent could comprise an electron lone pair, wherein that lone pair is capable of bonding to a proton or other cation; and wherein the lowest Singly-Occupied Molecular Orbital (SOMO) of RAD is lower in energy than a Doubly-Occupied Molecular Orbital (DOMO) of the neutral functional equivalent of NEG; and wherein the SOMO of RAD is higher in energy than the DOMOs of the neutral functional equivalent of NEG when the lone pair of the neutral functional equivalent of NEG is bonded to a proton or other cation.

For example, the neutral functional equivalent of NEG could be selected from the group: carbenes, stable carbenes (e.g. N-heterocyclic carbenes and diaminocarbenes), phosphines or amines. When the neutral functional equivalent is reacted with a proton (or other cation) the resultant group is positively charged. Such deactivation of the lone pair(s) can have an effect the stability of the radical.

Such neutral functional equivalents (of the anion of NEG) offer SOMO≠HOMO to SOMO=HOMO switching under quite different reaction conditions than the anion of NEG. For example, strong bases such as LDA (lithium diisopropylamide) are required to deprotonate stable carbenes, and these stable carbenes are known to reversibly coordinate to alkali metals such as lithium, sodium and potassium.

According to an embodiment of the invention, there is provided a method, use, process, structure or protecting group as defined previously, wherein the anion of NEG is not a chemical group but rather is a functional physical equivalent, which provides the same function as the anion of NEG. That is, the functional physical equivalent comprises a negative point charge, wherein that negative point charge is capable of being neutralized, and wherein the energy level of the unpaired electron of RAD is lowered when the functional physical equivalent of NEG together with RAD-L forms the (corresponding) structure of Formula (I), and wherein the energy level of the unpaired electron of RAD is the highest energy level when the functional physical equivalent is electrically neutralized.

The negative point charge could be simply electronically neutralized. For example, an electrode could comprise the negative point charge, and wherein a surface could comprise a plurality of such electrodes. Where the electrode comprises a negative point charge, that point charge could be neutralized by turning off the electrode or by reversing the polarity of the electrode. The skilled person would appreciate the ways in which an electrode could be used to manipulate the nature of such a negative point charge.

Also, for example the effect of the negative point charge may be tuned by the modification of the proximity of the point charge to RAD through space.

It is also conceived that the UV, IR, ESR and Mass Spectra will be different in the SOMO≠HOMO and SOMO=HOMO forms of the structure of Formula (I). Such differences could be used in the quantification of such species, detection of orbital conversion in them, and/or could be used to alter the range in which a functional group is normally detectable into a more useful detectable range.

According to an embodiment of the invention, there is provided a method, use, process, structure or protecting group as defined previously, wherein the structure of Formula (I) is selected from the group comprising:

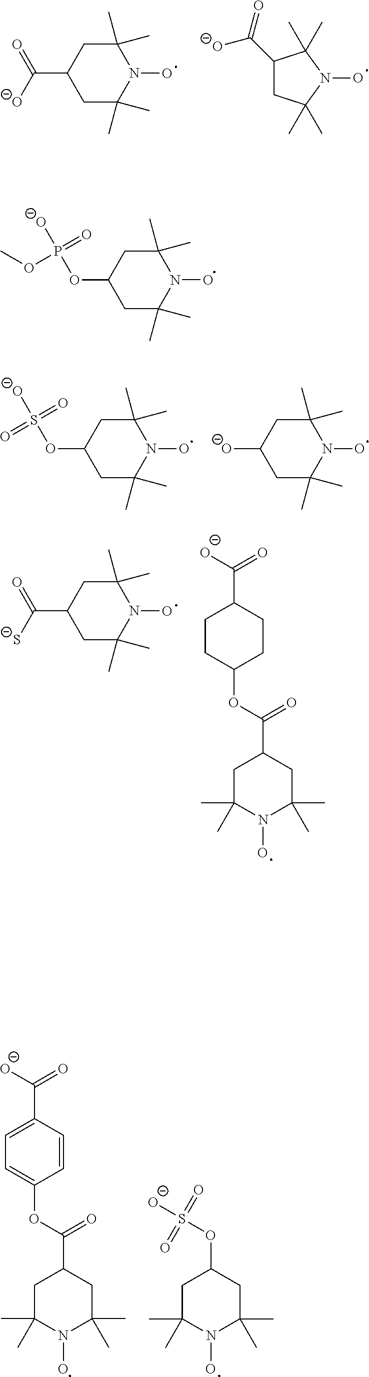

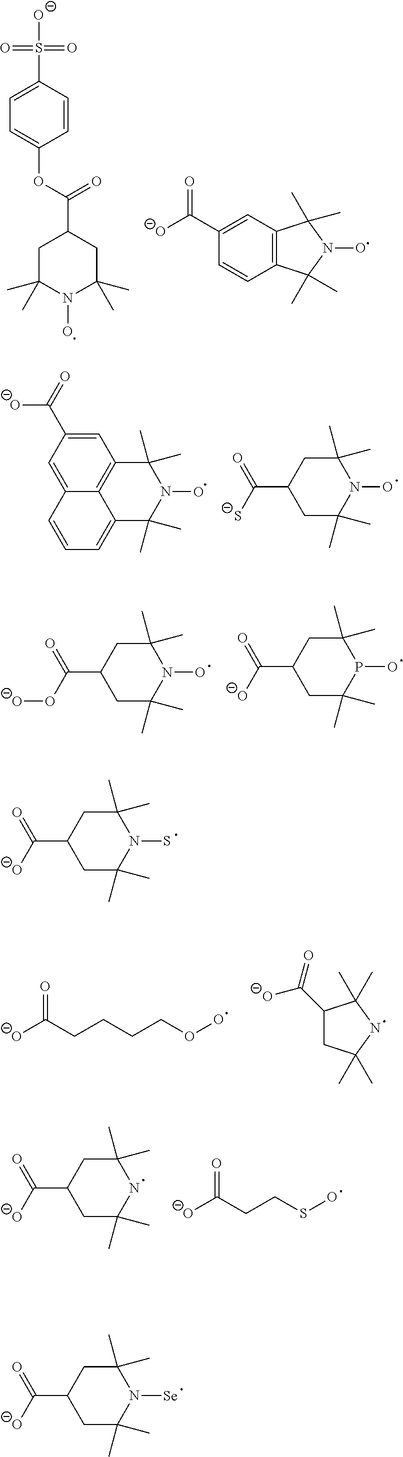

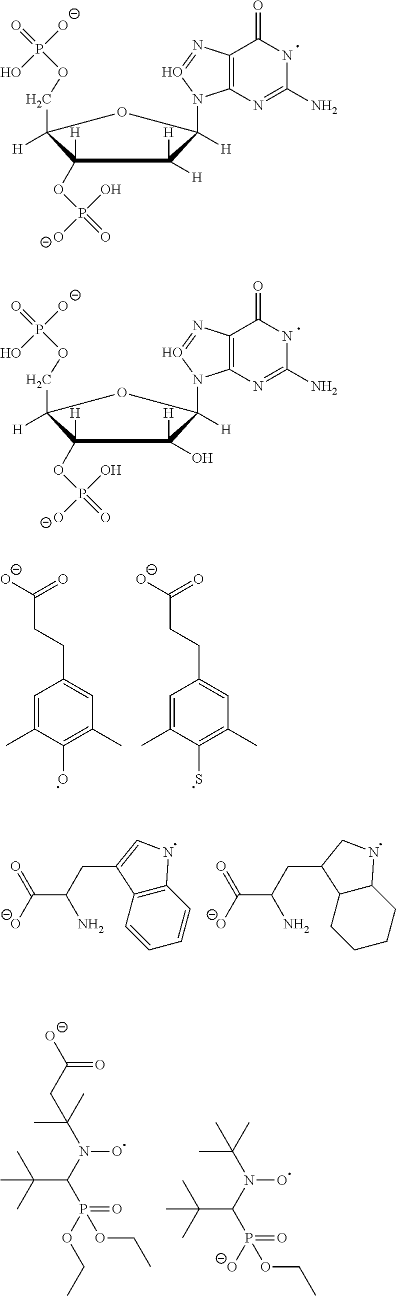

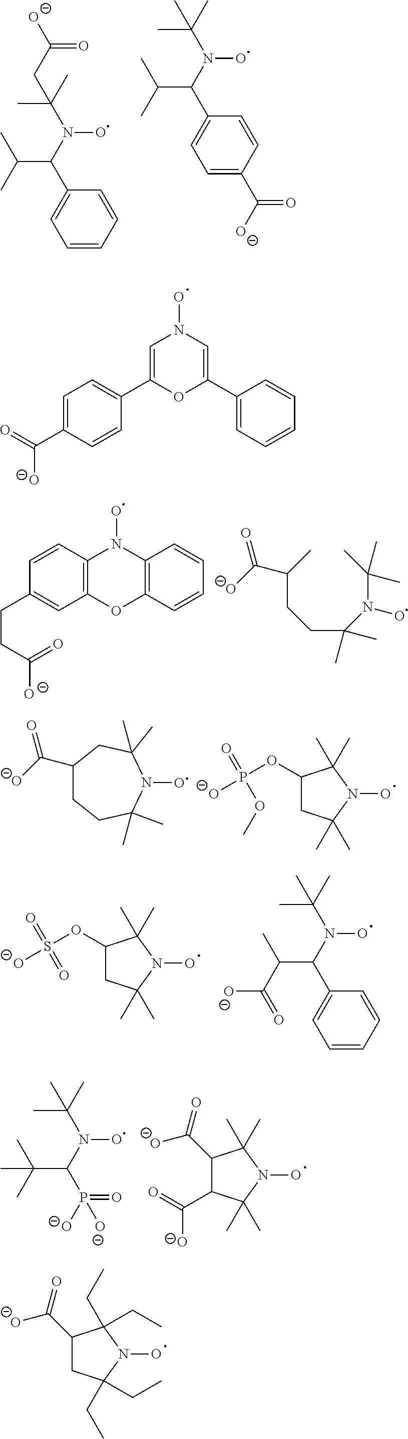

According to an embodiment of the invention, there is provided a method, use, process, structure or protecting group as defined previously, wherein the structure of Formula (I) is selected from the group comprising intermediates and/or products of the oxidative degradation of lipids, phospholipids, amino acids, peptides, nucleic acids or nucleotides.

According to an embodiment of the invention, there is provided a method, use, process, structure or protecting group as defined previously, wherein the structure of Formula (I) is selected from the group comprising the intermediates and/or products of the oxidative degradation of lipids, phospholipids, wherein such species comprise a structure selected from:

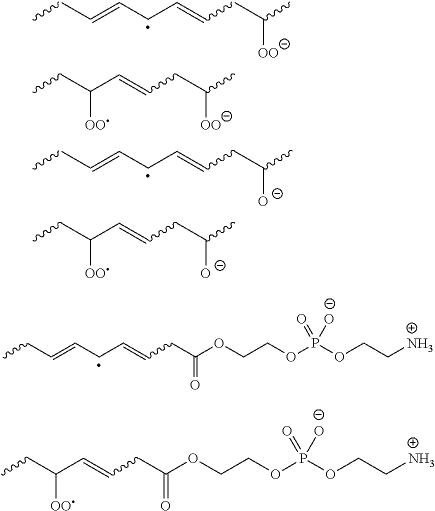

### BRIEF DESCRIPTION OF THE DRAWINGS

A preferred embodiment of the present invention will now be described, by way of an example only, with reference to the accompanying drawings wherein:

FIG. 1 is a schematic summary of the chemical aspects and molecular orbitals considered in the present invention.

FIG. 2 shows distonic radical anions with the orbital conversion.

FIG. 3 shows pH-switching of radical stability.

FIG. 4 shows the assessment of the σ-assistance effect.

FIG. 5 shows experimental evidence of the BDE-switching by pH-induced orbital conversion.

FIG. 6 shows SOMO-HOMO conversion in various substrates.

FIG. 7 shows a SEC traces of PMA-Br (▪), PMA-Br with HOOC-TEMPO.

FIG. 8 shows a SEC traces of PMA-Br (▪), PMA-Br with HOOC.

FIG. 9 shows a Gaussian simulation of PMA-Br with HOOC-TEMPO.

FIG. 10 shows SEC traces of purified PMA-ON—COOH.

FIG. 11 shows a Gaussian simulation of PMA-ON—COOH for 6 hours.

FIG. 12 shows a Gaussian simulation of PMA-ON—COOH for 20 hours.

FIG. 13 (a) shows a proposed mechanism for PFL (activated by PFL-EA) in the catalytic condensation of CoA and pyruvate to from acetyl-CoA and formate; (b) shows a summary of the overall transformation.

FIG. 14 shows an exemplary schematic potential energy profile of reactions with and without switching in the transition states and products.

FIG. 15 plots the pH switch in solution (various solvents) versus the pH switch in the gas phase.

FIG. 16 shows proof of pH switching in low polarity solution.

FIG. 17 shows a repeat of the experiment as detailed in FIG. 16 except that a base 1,8-diazabicylo[5.4.0]undec-7-ene (DBU, 33 nM) was included.

FIG. 18 shows pH switching in aqueous solution.

### DETAILED DESCRIPTION

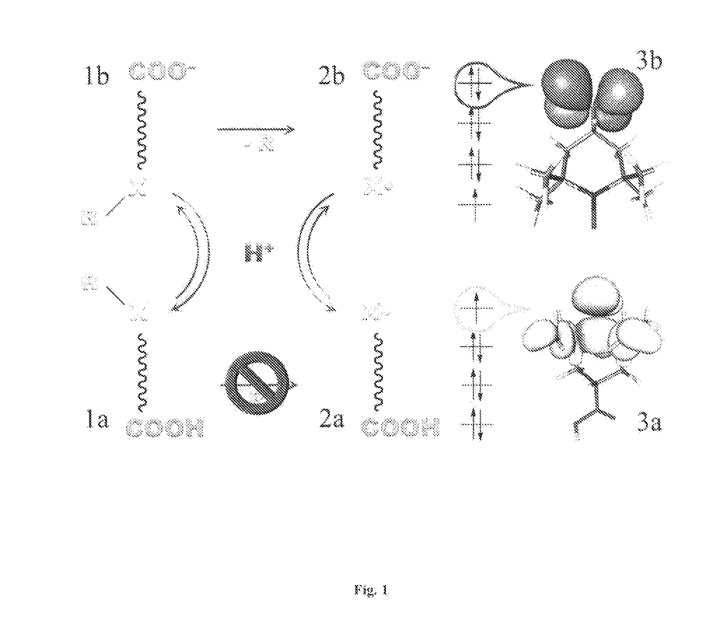

FIG. 1 shows a schematic summary of the chemical aspects and molecular orbitals considered in the present invention. Starting in the bottom left corner of the figure there is shown a molecule in which an acid group (COOH) is linked to a group X by a linker, and group X is bonded to a group R. The acid group broadly conforms to the NEG group of the invention, the linker structure broadly conforms to the L group of the invention and the X group (when in the unbound state, i.e. .X) corresponds to the group RAD.

It can be seen that when the molecule in the bottom left corner is protonated the molecule does not readily dissociate into the radical shown as .X—COOH. This is because the radical is not stable and would not readily dissociate. However, when a proton is removed from the acid a new species can form i.e. R—X—COO⁻. However, by contrast, R—X—COO⁻ can dissociate to give the radicals R. and .X—COO⁻, this is because the radical formed .X—COO⁻ is stabilised by the orbital ‘conversion’, i.e. wherein the singly-occupied molecular orbital is not the highest-occupied molecular orbital (i.e. SOMO≠HOMO). In that regard, a representation of the molecular orbitals is shown next to the .X—COO⁻ species, together with an energy level diagram of the orbitals. In that orbital diagram of .X—COO⁻ it can be seen that there is an unfilled orbital (i.e. shown as [symbol] ) which is not the highest orbital, in disconformity with the established aufbau principle. If a proton is then added to .X—COO⁻ species, a new species .X—COOH is formed. Again, a representation of the molecular orbitals is shown next to that species together with an energy level diagram of the orbitals. In this case it can be seen that an unfilled orbital (i.e. shown as +) has become the HOMO, and this is caused by the addition of a proton. That is, the proton has acted to change the electronic stricture of the species and hence it's chemistry.

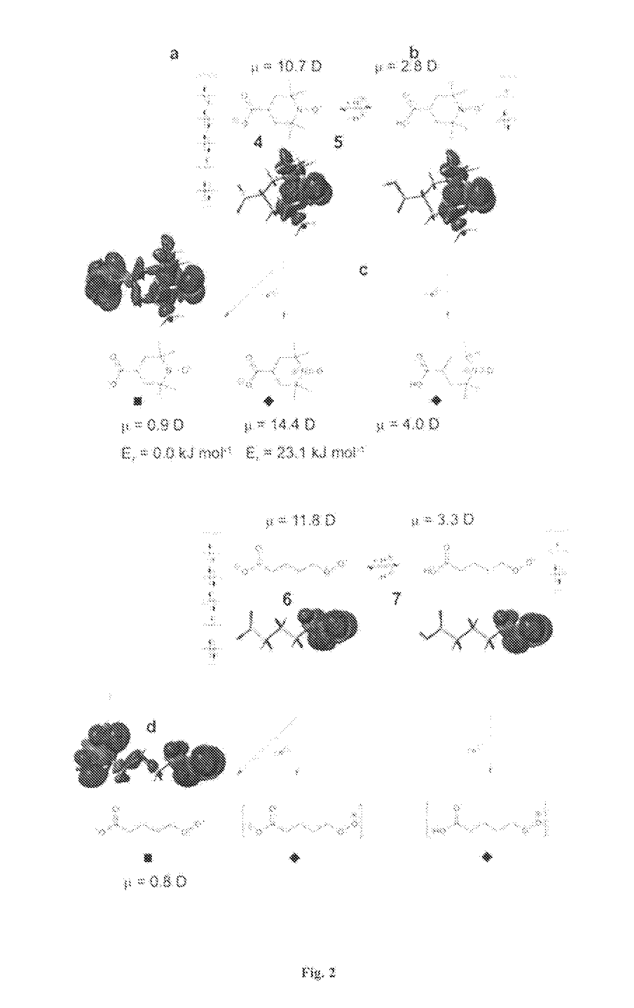

FIG. 2 shows distonic radical anions—the new class of species with the SOMO-HOMO conversion. Shown are M06-2X/6-31+G(d) optimized geometries, spin density plots (for open-shell species only) and dipole moments (μ, in Debye), along with the MCSCF(9,5)/6-31+G(d) molecular orbital configurations of the investigated carboxy-aminoxyl (a) and peroxyl (b) radicals in the switched (deprotonated) and non-switched (protonated) forms, as well as the closed-shell (♦) and triplet biradical (▪) products of their one-electron oxidation (c and d). Calculated relative energies (Eᵣ, in kJ mol⁻¹) are given for the oxidation products of distonic radical anions only. See Example 1 to Example 4 for the orbital plots and configurations, and see Example 6 for the detailed description of the oxidation products.

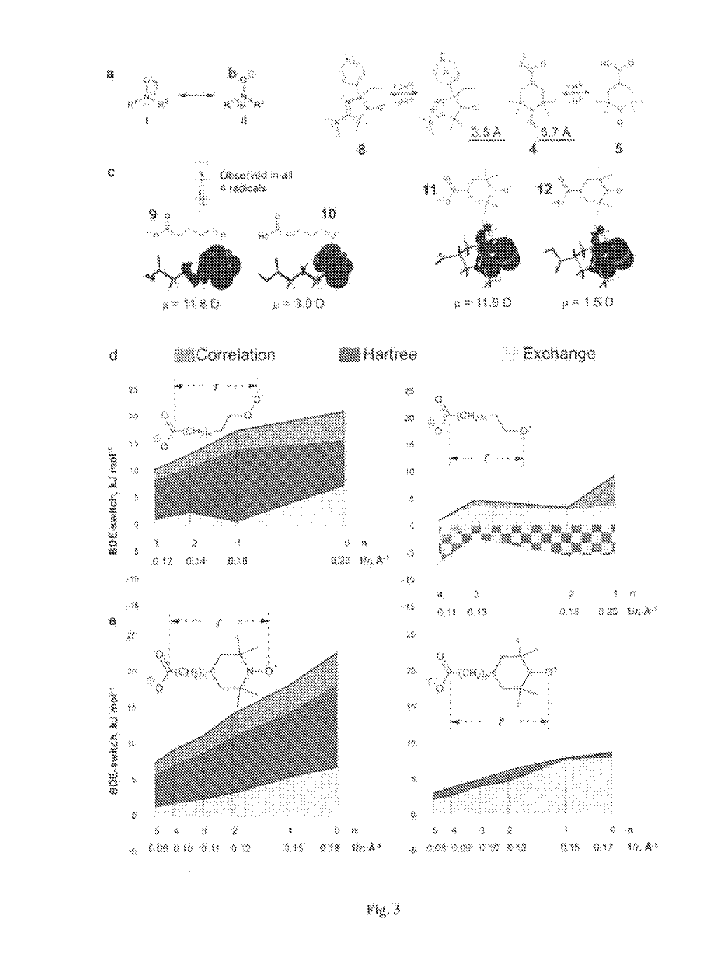

FIG. 3 shows pH-switching of radical stability. a, Two resonance forms of an aminoxyl radical. b, Switching of aminoxyl stability by pH-induced polar effects (the underlined numbers are the distances between the two coloured atoms in the M06-2X/6-31+G(d) optimized geometries, Å). c, Structures, M06-2X/6-31+G(d) spin density plots (for open-shell species only) and dipole moments (μ, in Debye), as well as MCSCF(9,5)/6-31+G(d) molecular orbital configurations of the reference radicals in protonated and deprotonated forms. d-e, Energy decomposition of the calculated BDE-switches (G4(MP2)-6X for acyclic series and ONIOM approximation for the cyclic series, gas phase, kJ mol⁻¹) in the pairs of homologue series of trial and reference radicals, plotted against the number of methylene units n and inverse of distance r between the radical centre and the carboxylic carbon; “Correlation” is the difference between total and Hartree-Fock energies, “Hartree” is the sum of Coulomb, kinetic and potential energy contributions to the HF energy, “Exchange” is the sum of alpha and beta exchange contributions to the HF energy.

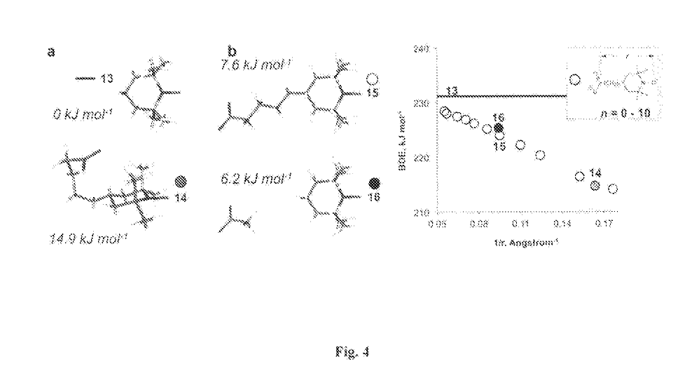

FIG. 4 shows assessment of the σ-assistance effect. a, M06-2X/6-31+G(d) optimized geometries and BDE-switches (in electronic energy terms, kJ mol⁻¹) of non-substituted TEMPO (—), carboxy-TEMPO n=4 homologues in extended chain (◯) and lowest energy ( [symbol] ) conformations, and the corresponding non-bonded TEMPO . . . CH₃COOH complex (). b, Calculated methyl BDEs (kJ mol⁻¹) of the species in a and full carboxy-TEMPO homologue series (extended chain conformers with n=0-10).

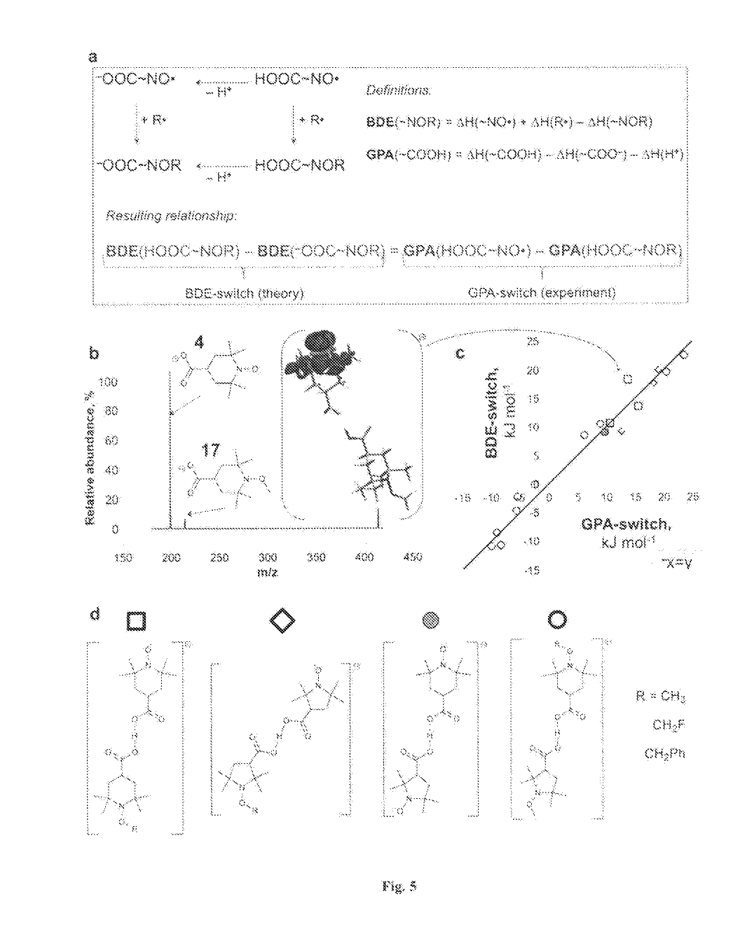

FIG. 5 shows Experimental proof of the BDE-switching by pH-induced orbital conversion. a, Thermocycle relating the difference in the bond dissociation energies (BDE) of the switched and non-switched aminoxyl radicals with the difference between their GPAs and the GPAs of the corresponding alkoxyamines; all reported quantities are in enthalpic terms. b, M06-2X/6-31+G(d) optimized geometry and spin density plot, as well as the CID negative-ion mass spectrum of the [AHB]⁻ dimer (A=carboxy-TEMPO 4 and B=carboxy-TEMPO-methyl 17) at the collision energy of 10 units. Optimized COO—H bond lengths in the dimer are equal to 1.33 Å for Å and 1.33 Å for B. c and d, Calculated BDE-switches (gas-phase, 25° C.) plotted against experimentally measured GPA-switches for carboxy-TEMPO vs. its alkoxyamines (□), carboxy-PROXYL vs. its alkoxyamines (⋄), carboxy-PROXYL vs. carboxy-TEMPO ( [symbol] ) and different combinations of alkoxyamines (◯, only one example is shown), for details see Examples).

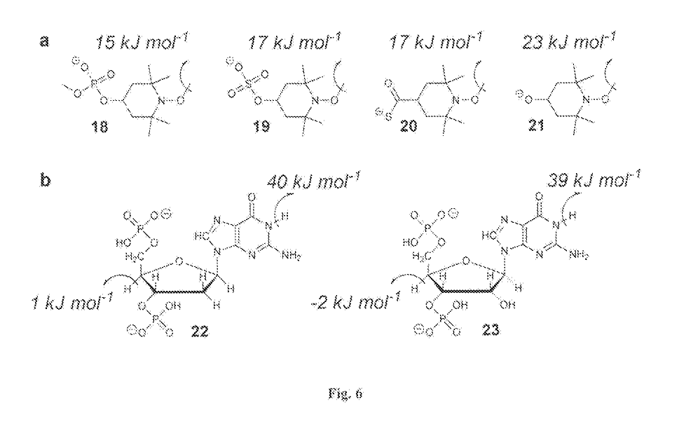

FIG. 6 shows SOMO-HOMO conversion in various substrates. Calculated BDE-switches (italics, in electronic energy terms, kJ mol⁻¹) in TEMPO-CH₃ substituted with different anionic groups (a, using G3(MP2,CC)(+) method) and in model DNA 22 and RNA 23 sugar and base (guanine) moieties (b, using ONIOM approximation to the G3(MP2)RAD energies at the MP2/6-311+G(3df,2p) level of theory); see Table 14 and Table 15 for details.

FIG. 7 shows a SEC traces of PMA-Br (▪), PMA-Br with HOOC-TEMPO reaction in Toluene with CuBr/PMDETA for 30 min (♦), and 20 h () at R.T.

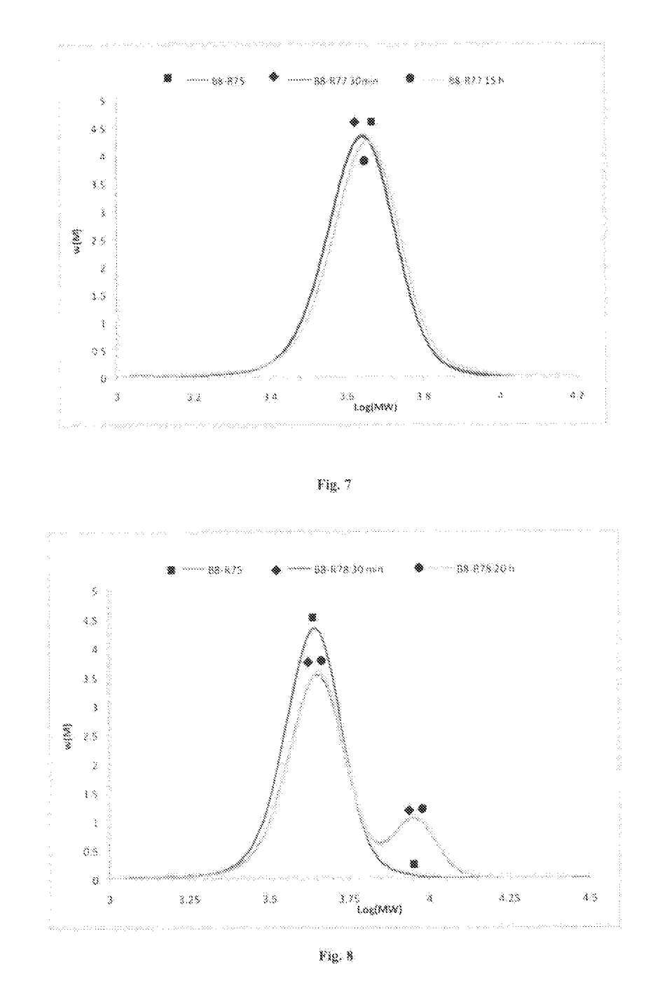

FIG. 8 shows a SEC traces of PMA-Br (▪), PMA-Br with HOOC-TEMPO reaction in DMSO with CuBr/Me₆TREN for 30 min (♦) and 20 h () at R.T.

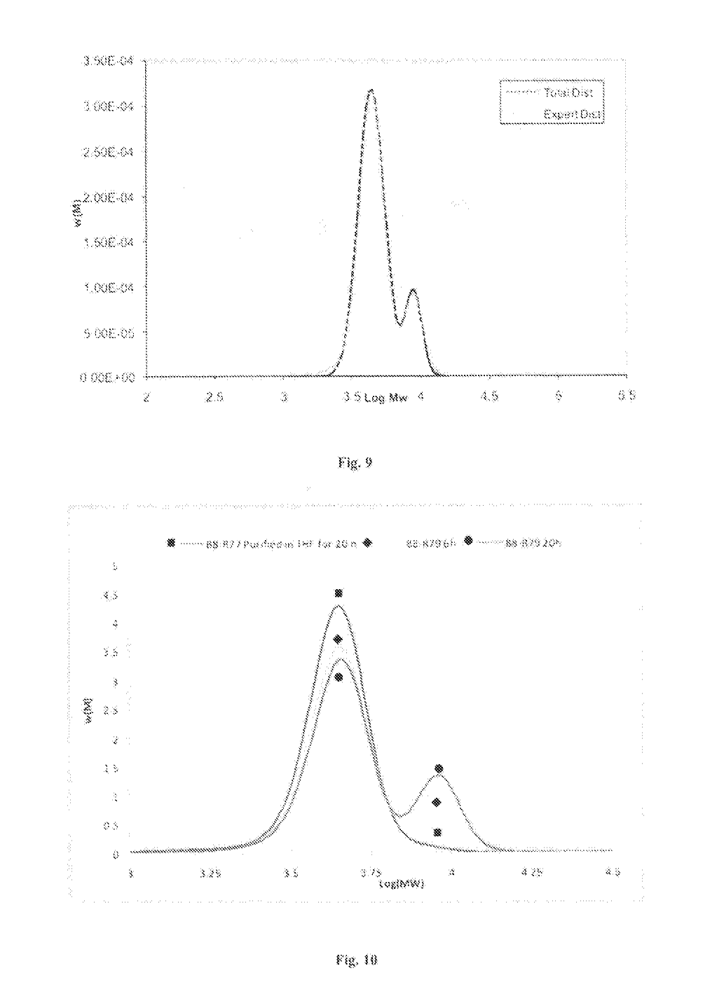

FIG. 9 shows a Gaussian simulation of PMA-Br with HOOC-TEMPO reaction in DMSO with CuBr/Me₆TREN (-) and LMD simulation (--). It showed 27.3% of coupling product. Simulation: peak 1: Mₚ=4454 PDI=1.05. (PDI used here is lower than the linear PMA-Br 1.06 to make a better fitting). peak 1: Mₚ=8941 PDI=1.02.

FIG. 10 shows a SEC traces of purified PMA-ON—COOH kept in THF overnight (♦), PMA-ON—COOH in DMSO with Me₆TREN for 6 h () and 20 h (▪) at R.T.

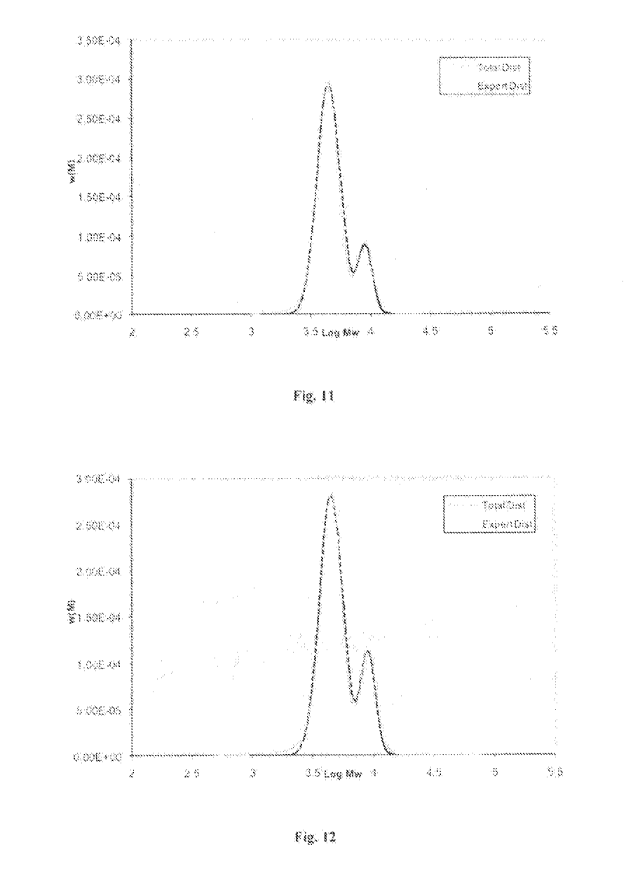

FIG. 11 shows a Gaussian simulation of PMA-ON—COOH in DMSO with Me₆TREN for 6 h (-) and LMD simulation (--). It showed 27.2% of coupling product. Simulation: peak 1: Mₚ=4511 PDI=1.05. (PDI used here is lower than the linear PMA-Br 1.06 to make a better fitting). peak 1: Mₚ=9088 PDI=1.02

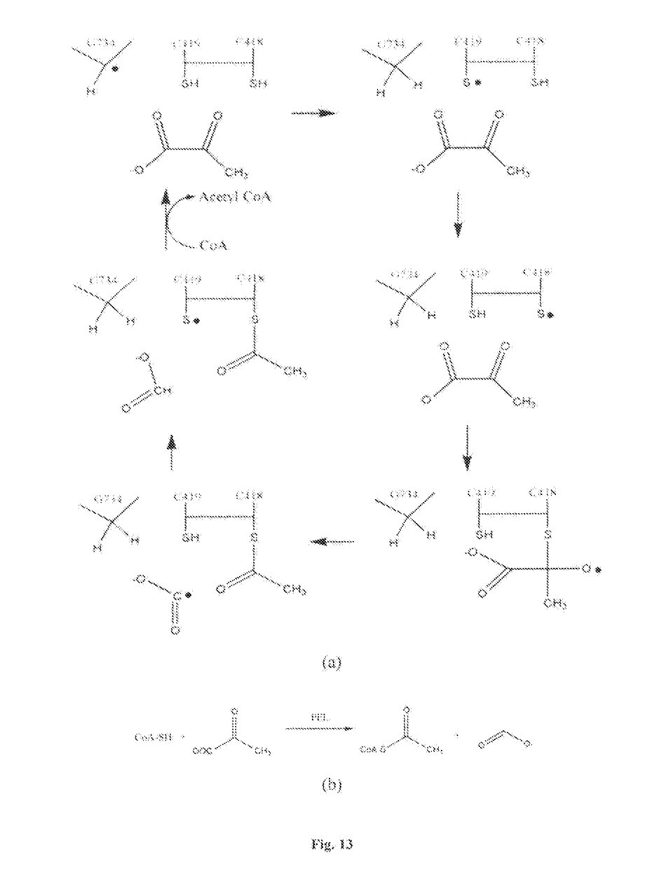

FIG. 12 shows a Gaussian simulation of PMA-ON—COOH in DMSO with Me₆TREN for 20 h (-) and LMD simulation (--). It showed 33.3% of coupling product. Simulation: peak 1: Mₚ=4511 PDI=1.05. (PDI used here is lower than the linear PMA-Br 1.06 to make a better fitting). peak 1: Mₚ=9088 PDI=1.02.

FIG. 13 shows (a) a proposed mechanism for the condensation of CoA and pyruvate to acetyl-CoA and formate, an important reaction in the anaerobic glucose metabolism of bacteria. The process shown occurs via a radical mechanism—homolytic cleavage of the C1-C2 bond in pyruvate. Without being bound by theory, it is proposed that before PFL can begin catalysis, it must be activated by PFL-AE (a 28 kDa monomeric enzyme, a member of the radical S-adenosylmethionine family), it is thought that the active site of PFL-AE contains a [4Fe-4S]+ cluster which reduces G734 on PFL via an S-adenosylmethionine (AdoMet) cofactor. It is thought that the PFL-AE stereospecifically abstracts H from PFL G734's Ca to form a glycyl radical (the active form of PFL) which can then react with pyruvate via active site cysteine residues C419 and C418. It is thought that then the pyruvate reacts to form an acetyl-enzyme intermediate, releasing pyruvate; the acetyl-enzyme intermediate is then attached by CoA to give acetyl-CoA. It is thought that the carboxylate group of pyruvate (e.g. corresponding to the anion of NEG) can stabilise the radical on G734 (e.g. corresponding to the radical of RAD) to give a structure of Formula (I), where the radical and anion are sufficiently close (i.e. proximate) to have a through-space interaction to give the SOMO≠HOMO configuration. Alternatively, or in addition, it is also thought that the carboxylic group (e.g. corresponding to the anion of NEG) of the aspartate residue of the D16 group of PFL-AE (not shown in FIG. 13) can stabilise the radical on G734 (e.g. corresponding to the radical of RAD) to give a structure of Formula (I), where the radical and anion have a through-space interaction to give the SOMO≠HOMO configuration; (b) shows the overall reaction of the catalytic system shown in (a), where CoA and pyruvate are converted to Acetyl-CoA and formate.

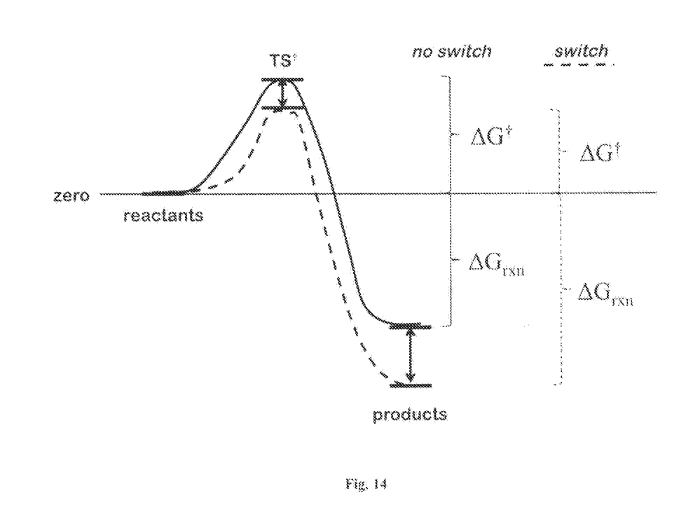

FIG. 14 shows an exemplary schematic potential energy profile of reactions with and without switching in the transition states and products, wherein ΔS is change of entropy; ΔEₑ is change of electronic energy; Δ(Eₑ+ZPVE) is change of electronic energy corrected for zero-point vibration (enthalpy at 0 K); ΔH is change of enthalpy (previous term including thermal correction, or enthalpy at a given temperature); ΔG is a change of Gibbs free energy (energy including thermal and entropic contributions at a given temperature); † denotes activation (kinetic) parameters, and r×n denotes reaction (thermodynamic) parameters. Depending on whether the species with orbital conversion are reactants or products of the reaction and whether its transition state has an early or a late character the activation barrier ΔG^{†} of such reaction can be affected by the switching. In this example, it is larger in the case of non-switched species compared to the switched species forming in the reaction.

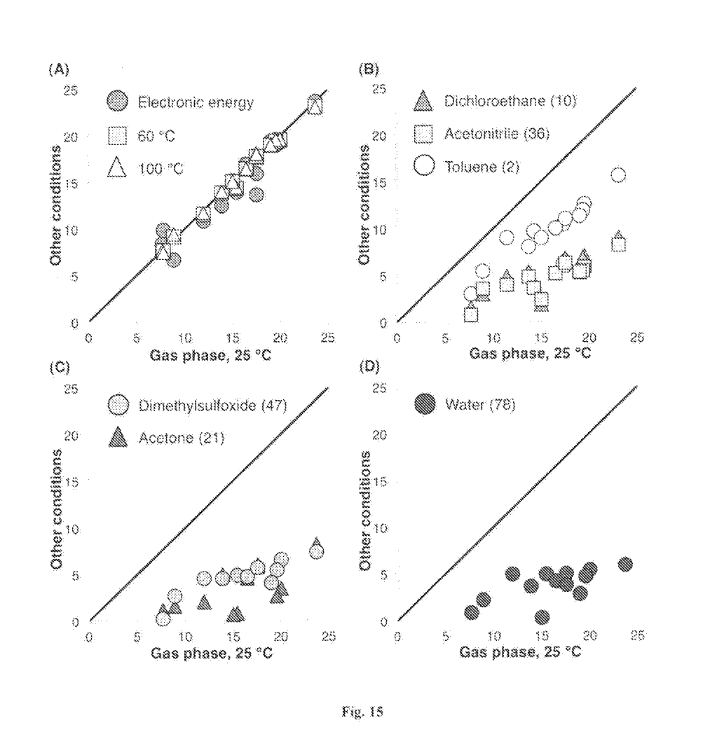

FIG. 15 plots the pH switch in solution (various solvents) versus the pH switch in the gas phase for a big test set of species. The pH switches were obtained using accurate quantum chemistry. Details of the species represented in this figure are provided in Table 21. These quantum chemical calculations reveal that the presence of polar solvents reduces the underlying stabilizing effect substantially, which is consistent with its electrostatic origin. Nonetheless they show clearly that substantial pH switches are retained in low polarity solvents, and that the switches in polar solvents, while smaller, do remain synthetically useful for some combinations of leaving groups where solvent interactions help to favour dissociation in deprotonated versus protonated form.

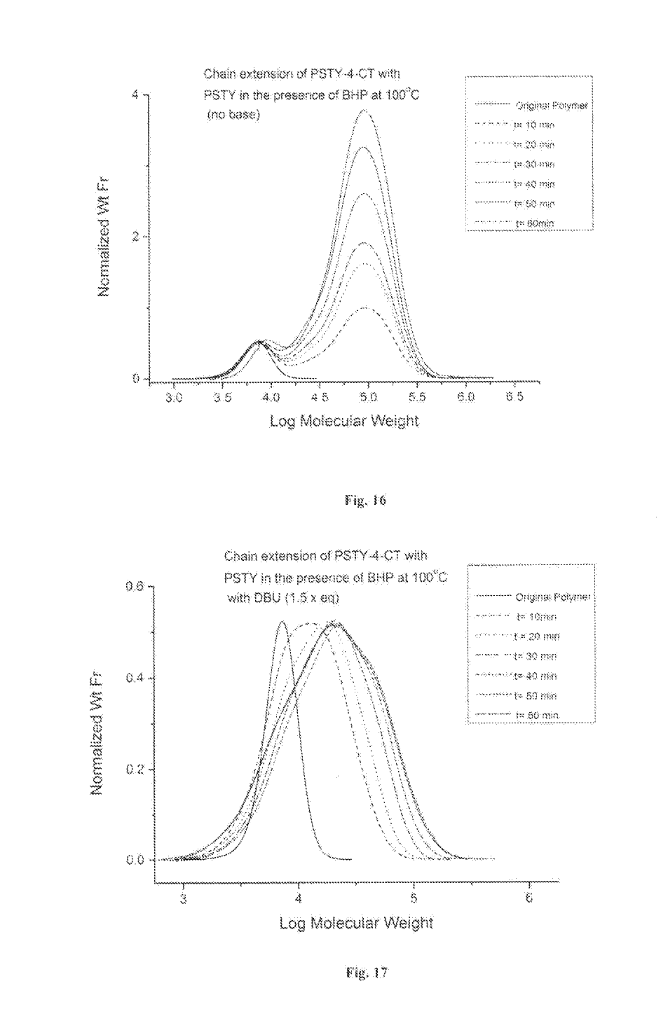

FIG. 16 shows proof of pH switching in low polarity solution (in this case bulk styrene monomer). The plot shows the molecular weight distribution of a polymeric alkoxyamine comprising a polystyryl chain bound to a 4-carboxy-TEMPO nitroxide (11.6 mg/mL) as it is heated to 100° C. in the presence of a free-radical initiator tert-butyl hydroperoxide (BHP) and styrene monomer but no base. Experiments were run under nitrogen. Under those conditions the carboxylic acid remains protonated. It is clear that the original alkoxyamine remains stable at that temperature (i.e. the original peak is unchanged), while an independent new conventional free-radical polymerization (producing the second higher molecular weight peak that grows with time) takes place.

FIG. 17 shows an exact repeat of the experiment as detailed in FIG. 16 except that a base 1,8-diazabicylo[5.4.0]undec-7-ene (DBU, 33 nM) was included to deprotonate the carboxylic acid of the 4-carboxy-TEMPO. It is clear that this system behaves very differently. Instead of the original polymer remaining intact and a new independent polymer growing, the entire molecular weight distribution of the polymer shifts to increasingly higher molecular weight. This is indicative of the original alkoxyamine undergoing dissociation so that the resulting free styryl radical can add to monomer and grow, and evidence that the deprotonation triggers dissociation.

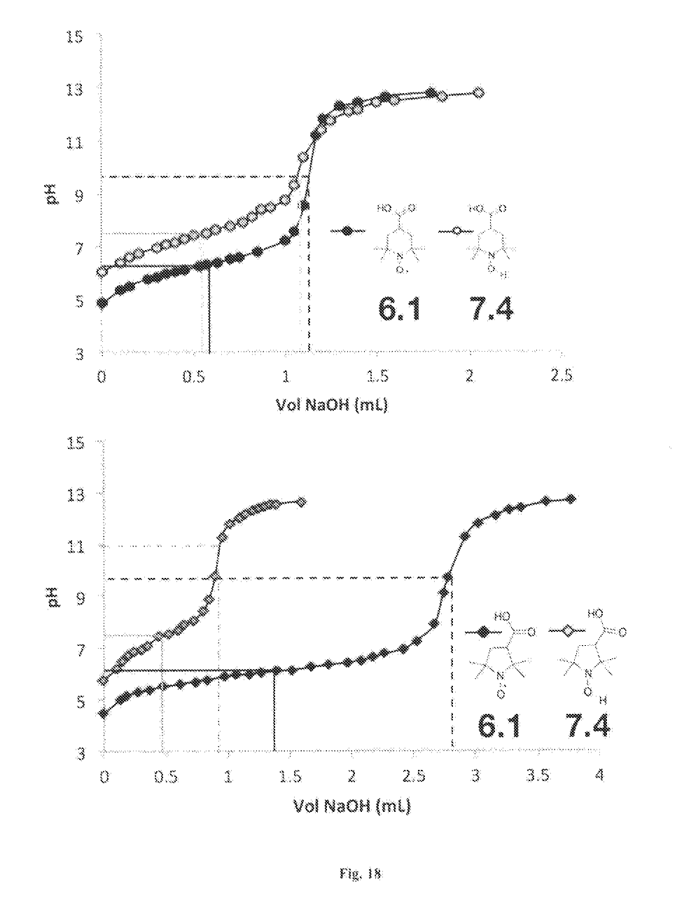

FIG. 18 shows proof of pH switching in aqueous solution. In this case, a standard potentiometric titration was used to measure the pKa of 4-carboxy-TEMPO nitroxide and various non-radical forms of it (i.e. alkoxyamines and hydroxylamine) to show that formation of the radical leads to increased acidity of the carboxylic acid group. In the example shown, the hydroxylamine has a pH switch of 1.5 pKa units in a 75% ethanol/25% water solvent. The pKa switch corresponds directly to a switch on radical stability of 1.5 orders of magnitude. In another example not shown but carried out under identical conditions, a pKa switches of 0.5 unit were determined for 4-carboxy-TEMPO with a benzyl leaving group. The smaller switch for this leaving group is due to the differing solute-solvent interactions which are less favourable for dissociation of the deprotonated alkoxyamine versus protonated.

It has been surprisingly found that a structure comprising Formula (I) as defined above, has very different chemical properties depending on whether the anion of NEG is protonated (or bonded to a cation) or not.

It has been surprisingly found that when the anion of NEG is not bonded to a proton or other cation, the singly-occupied molecular orbital, that is, the molecular orbital containing the radical, is not the highest-occupied molecular orbital (i.e. the SOMO≠HOMO). This means that the singly-occupied molecular orbital is lower in energy than one or more of the fully-occupied molecular orbitals.

As mentioned above, this electronic arrangement runs contrary to the established aufbau principle of molecular orbital filling, where it is usual for the SOMO to be the highest-occupied molecular orbital i.e. SOMO=HOMO.

However, in the present invention, it has also been found that when the anion of NEG in the structure of Formula (I) is bonded to a proton or other cation, the molecular orbitals can be changed to the more conventional electronic ordering in accordance with the aufbau principle, and as such, the SOMO becomes the highest-occupied molecular orbital i.e. SOMO=HOMO.

In this way, the present invention provides a way in which the electronic configuration of the structure of Formula (I) can be switched between the SOMO≠HOMO and SOMO=HOMO configurations. Importantly, this switching of the orbitals can be achieved by simple chemical means, and is reversible.

The changing of these orbital configurations in this way has a profound effect on the chemical properties of the resultant species.

In the SOMO=HOMO arrangement, the reactivity of that species will be dominated by radical reactions, whereas in the SOMO≠HOMO arrangement reaction involving the radical will be suppressed, if not substantially quenched.

In that way, the present invention allows the molecular orbitals in the structure of Formula (I) to be ‘switched’ from one electronic configuration to another physically different configuration, and this being brought about by the simple addition or removal of protons or other cations. This change in electronic configuration fundamentally changes the nature of the chemistry of the resultant species. Direct access to the ‘switching’ of molecular orbitals in this way by direct chemical means has hitherto been unknown. This work has been published in Nature Chemistry (Gryn'ova G., Marshall D., Blanksby, S J, Coote M. L. Nature Chem. (2013), 5, 474-481) and is incorporated herein by reference.

The present invention provides for the structure of Formula (I) to be tailored in various ways to allow modification of the resultant reactivity of the ‘normal’ (SOMO=HOMO) and ‘converted’ (SOMO≠HOMO) electronic configurations.

The properties of Formula (I) that can be modified comprise:

### (i) Energy Differences in SOMO≠HOMO and SOMO=HOMO Configurations

The energy difference between states when the SOMO=HOMO as compared to the state where SOMO≠HOMO, is indicative of the difference in the reactivity of the two species having those states.

The larger the magnitude of that energy difference, the greater the expected difference in the chemical properties of those switchable species. The magnitude of that energy difference can be manipulated by the arrangement of the various groups in Formula (I). For example, this can be changed by changing the RAD, L and NEG groups, in terms of the stability of those groups, their connectivity and their spatial arrangement in three dimensional space. In that way the difference between energy states when the SOMO=HOMO as compared to the state where SOMO≠HOMO can be manipulated, and so the resultant chemical properties of the ‘normal’ and ‘converted’ species can be changed. The resultant chemical properties of the ‘normal’ and ‘converted’ species can be tuned to be sensitive to the reaction conditions or environment in which they are to be used.

### (ii) Binding of the Anion of NEG to Protons and Cations

The binding nature of the anion of NEG to protons (and to other cations) also allows further manipulation of the properties of the structure of Formula (I). The various classes of anions will be known to the skilled person.

For example, weakly acidic NEG groups (or strongly basic groups) will tend to bond protons (and by extension other cations) more strongly, whereas more acidic NEG groups (or weakly basic groups) will tend to bond protons (and other cations) more weakly.

As mentioned above, when the anion of NEG is not bonded to a proton or bonded to a cation then electronic structure of Formula (I) will be in the ‘converted’ arrangement (i.e. SOMO≠HOMO); whereas when the anion of NEG is bonded to a proton or other cation, the electronic configuration is in the more ‘normal’ aufbau electronic configuration (i.e. SOMO=HOMO).

In that way the chemical properties of the ‘normal’ and ‘converted’ species can be tuned to be sensitive to the reaction conditions in which they are to be used.

More specifically, the nature of the anion of NEG can allow access to the control of the ‘switch’ that causes the interconversion of the ‘normal’ and ‘converted’ arrangements. Therefore, the anion can be selected to be responsive to certain reaction conditions, or could be tailored to bind more selectively to one cation in preference to another. This could be for example used to allow the switching from the ‘normal’ electronic configuration to the ‘converted’ electronic configuration to occur in a certain pH range.

Therefore, the anions of NEG that bond strongly to protons or other cations will offer different reaction profiles to those that do not bond strongly, allowing the anions to be tuned to suit the reaction conditions. In addition, reaction conditions like the polarity of the solvent will affect how strongly the anion of NEG will bond to protons and/or other cations (such as metal ions and quaternary amines). These reaction conditions, in combination with the nature of the anion of NEG, could be used to moderate the reactivity of the structure of Formula (I). In particular these could be used to control the switching between SOMO≠HOMO and SOMO=HOMO states.

(iii) RAD and the Acidity of the Anion of NEG

The structure of Formula (I) offers the nature of the radical of RAD to be tuned. Various radical types are known, and the stability of these groups varies. The various classes of radicals will be known to the skilled person.

The radical of RAD can be more or less stable, and this stability can be used to affect the magnitude of the energy difference between the SOMO orbitals in the ‘normal’ and ‘converted’ configurations.

Moreover, the invention also provides access to the manipulation of the acidity of the anion of NEG through the control of nature of the radical.

It has been surprisingly found that the nature of the radical can be used to influence the acidity of the anion of NEG (for example where the anion is a carboxylic acid).

In the ‘converted’ electronic arrangement, the stability of the resultant radical of RAD is found to affect the acidity of the (acidic) anion of NEG. As the radical of RAD is stabilised, there is a greater propensity for the protons associated with the anion of NEG to then dissociate. Where the radical of RAD is less stabilised by the structure of Formula (I), the protons associated with the anion of NEG are less likely to dissociate.

That is, the anion of NEG (i.e. the conjugate base of the acid) has a pK_{b} which is lower than the corresponding structure where the radical is not present, because it is involved in bond forming. The pKₐ of the protonated anion of NEG (i.e. the conjugate acid of NEG) will correspondingly be higher in the same fashion.

Accordingly, the NEG group in Formula (I) is more acidic in nature as compared to the corresponding structure where the radical is not present, and the more stable the resulting radical the more acidic the resultant anion of NEG will be.

In that way, the binding properties of the anion of NEG can be manipulated by the nature (and/or inclusion) of the radical of RAD. For example, the inclusion of a radical into an acid bearing structure could be used for the purpose of increasing that acid's acidity, where the other requirements of the structure of Formula (I) are satisfied.

### (iv) Linker L

The nature of the linker L allows further manipulation of the structure of Formula (I).

For example L could be a group selected to provide certain physical properties to the structure of Formula (I), for example to give the resulting structure a certain solubility profile (e.g. increased solubility in non-polar solvents by the inclusion of fatty acid chains).

In addition, the nature of L can be used to affect the electronic properties of the structure of Formula (I)

L can be used to define the spatial arrangement of RAD and NEG in Formula (I), both in terms of connectivity and in terms of a three dimensional spatial arrangement.

This spatial arrangement of groups RAD and NEG can be used to change the magnitude of the difference between the energy states of SOMO=HOMO as compared to the SOMO≠HOMO for the radical of RAD. Without being bound by theory, it is believed that the radical of RAD and the anion (i.e. the negative charge) of NEG engage in a long range interaction which provides the effect which stabilises the radical of RAD, and so gives the SOMO≠HOMO electronic configuration. As such, as long as the radical of RAD and the anion (i.e. the negative charge) of NEG engage in that long range interaction, L could be space i.e. having a through-space interaction between RAD and NEG causing orbital conversion and related effect on RAD stability does not require a chemical link between RAD and NEG. In such situations, there may exist an equilibrium between the species, where RAD and NEG form the structure of Formula (I) and where these are not sufficiently close to form the structure of Formula (I). Such an equilibrium could be altered by changing the concentration of RAD or NEG, or how closely these two associate (e.g. changing the polarity of the medium which they are in). For example, where RAD and NEG are hydrophilic, these will tend to associate in non-polar solvents. Hydrogen bonding or other non-covalent means may be used to promote the association of RAD and NEG together, thereby bring these statistically into closer association, leading to an increase formation of the structure of Formula (I).

Moreover, the nature of L can be used to stabilise (or destabilise) the radical of RAD and/or to destabilise (or stabilise) the anion of NEG. In that way the magnitude of the difference between the energy states of SOMO=HOMO and SOMO≠HOMO can be further manipulated. Without being bound by theory, it is thought that stabilising the radical of RAD, and destabilising the anion of NEG can be employed to give the largest difference between the energy states of SOMO=HOMO and SOMO≠HOMO for the radical of RAD.

That stabilisation (or destabilisation) of the radical of RAD and/or the destabilisation (or stabilisation) of the anion of NEG could be brought about by steric or electronic effects.

Various groups could be introduced into L which could be situated proximate (i.e. close enough to have an effect) to RAD and NEG. These groups can be selected to have a steric and/or an electronic impact on the RAD and/or NEG groups. For example, one or more steric groups could be used to sterically protect a group, or these could be used to ‘twist’ a group into an unfavourable position destabilising the RAD and/or NEG groups, or bring two mutually incompatible groups into closer proximity. Electronic effects that could be used include delocalisation, π-conjugation, σ-assistance, hyper-conjugation, aromaticity and long range polar effects. Application of these means would be appreciated by the skilled person.

### (v) Reactivity of the Radical of RAD

Due to the resultant extra stability of the ‘converted’ radical, this radical is less likely to participate in bond forming reactions, and by extension, is more likely to (homolytically) cleave from bonds which it is participating in.

Accordingly, a radical of Formula (I) which has the ‘normal’ SOMO=HOMO arrangement and where that radical is bound to a radical to be protected, and could be released from that radical to be protected by causing the ‘normal’ SOMO=HOMO configuration to be switched to the ‘converted’ configuration (i.e. SOMO≠HOMO), by the simple removal of protons (or cations) from the anion of the NEG group.

In the reverse fashion, the stabilised SOMO≠HOMO radical structure can be made less stable by the addition of protons or other cations to the anion of NEG. These cations switch the orbitals from SOMO≠HOMO to the more ‘normal’ (but less stable) SOMO=HOMO arrangement. In that way the radical becomes destabilised and so more reactive, and thereby is activated to participate in bond forming reactions, and so could be ‘activated’ to react with a structure to be protected.

It can be seen that the addition of protons (or other cations) to the structure of Formula (I) causes the radical to be ‘activated’ and so to become more reactive and so that it can form a bond to a radical to be protected. Reversing the above process, i.e. removing protons or other cations stabilises the structure of Formula (I), and if suitably stabilised the stabilised radical can dissociate from the radical structure (i.e. by homolytic cleavage); or at least the process and/or conditions of removing the structure of Formula (I) from the structure to be protected can be made more moderate (e.g. using lower temperatures).

In that way, the structure of Formula (I) can be used as a (reversible) protecting group in chemical synthesis and/or analysis, as indicated in the aspects of the invention.

For example, a portion of a structure can be protected with the structure of Formula (I) e.g. R-RAD-L-NEG, while various other reactions are conducted on the unprotected portions of that structure. When required, the structure of Formula (I) can be selectively released (e.g. by increasing the pH) from that structure, allowing that portion which had been protected to be unprotected e.g. to give R. and RAD-L-NEG (where NEG bonds to a proton or other cation).

The radical to be protected, once deprotected, is free to participate in further reactions. The skilled person will be aware of those reactions, typically being radical type reactions.

A radical of Formula (I), in which the SOMO is lower in energy than one or more DOMOs is said to be SOMO-HOMO converted. This radical can be switched between its SOMO-HOMO converted form (SOMO≠HOMO) and the “normal” orbital configuration (i.e., in which the SOMO is higher in energy than all DOMOs and is thus the highest occupied molecular orbital or HOMO, SOMO=HOMO) by the addition of protons (or other cations) to the NEG group. In its SOMO-HOMO converted form (SOMO≠HOMO), the radical is more stable (and less reactive) than its non-SOMO-HOMO converted form (SOMO=HOMO). Thus, it is possible to switch the stability and reactivity of the radical by switching the orbital arrangement, which, as mentioned above, can be achieved by, for example, the addition of protons (or other cations) to the NEG group.

This switching of radical stability (i.e. from SOMO≠HOMO to SOMO=HOMO) can be seen in the bond energies that a radical of Formula (I) forms with other radicals, e.g. R.

The energy required to homolytically cleave the compound R-RAD-L-NEG into R. and RAD-L-NEG can be changed by switching between the SOMO-HOMO converted form (SOMO≠HOMO) of RAD-L-NEG and the normal form (SOMO=HOMO; where NEG is bonded to a proton or other cation).

In the SOMO-HOMO converted form (SOMO≠HOMO), the homolytic bond cleavage of R-RAD-L-NEG into the radicals RAD-L-NEG and R. will be more readily achieved.

In its non-SOMO-HOMO converted form (SOMO=HOMO) the radical of Formula (I) where NEG is protonated (or bonded to a cation) will more readily trap a radical species R. to form the compound R-RAD-L-NEG (where NEG is bonded to a proton or cation).

These switchable changes in bond dissociation energies are directly related to the equilibrium constants between R-RAD-L-NEG and the dissociated products R. and RAD-L-NEG. They also affect the dissociation rates in the same direction. Thus, the rate and extent of dissociation of R-RAD-L-NEG into R. and RAD-L-NEG can be enhanced by removing a proton or other cation from NEG so that RAD-L-NEG exists in its SOMO-HOMO converted form (SOMO≠HOMO). To reverse this process and cause RAD-L-NEG to trap R. one can simply protonate (or add another cation) to NEG to form its non-SOMO-HOMO converted form (SOMO=HOMO) that is less stable (hence more reactive) as a radical.

Thus, in its non-SOMO-HOMO converted form (SOMO=HOMO) a compound of Formula (I), where NEG is protonated or bonded to a different cation, can be used to “protect” R. and prevent it from undergoing other types of radical reactions through covalently bonding to RAD-L-NEG (where NEG is bonded to a proton or other cation) to give R-RAD-L-NEG (where NEG is bonded to a proton or other cation). To release R. and RAD-L-NEG one simply removes the added protons (or cations) from NEG group.

For any given combination of R. and RAD-L-NEG the extent of dissociation of R-RAD-L-NEG also depends on the temperature and the ability of other functional groups in R. and RAD to stabilize the respective radicals. When these are controlled for, switching between the non-SOMO-HOMO converted (SOMO=HOMO) and SOMO-HOMO-converted form (SOMO≠HOMO) allows a given equilibrium constant for dissociation of R-RAD-L-NEG to R. and RAD-L-NEG to be significantly increased. This also means that the temperature required to dissociate R-RAD-L-NEG can be lowered compared with corresponding non-SOMO-HOMO converted compounds.

In view of the tunability and adaptability of the structure of Formula (I), it is possible for example that more than one protecting group of the invention could be used on the same structure to be protected, whereby each protecting group is deprotected under different reaction conditions, for example each responsive in a certain pH range or other cation concentration range, or to progressively harsher deprotecting conditions such as increasing temperature. It is also possible that more than one protecting group can be removed (from a structure to be protected) under the same conditions. When two radical protecting groups are removed, the two resulting radicals on the same structure can be arranged such that they can recombine. The two resultant radicals could be arranged such that the cyclization of the structure bearing these radicals occurs when they are deprotected (e.g. an intramolecular Wurtz reaction).

### EXAMPLES

The invention will be discussed with reference to calculations, methods and experimental data presented below. The calculations (in part) have been published in Nature Chemistry (Gryn'ova G., Marshall D., Blanksby, S J, Coote M. L. Nature Chem. (2013), 5, 474-481) and is incorporated herein by reference.

The present invention provides new SOMO-HOMO converted compounds with an alternative source of high-energy HOMO(s) that enables the switching between the ‘regular’ and ‘converted’ orbital configurations.

Using in part quantum chemistry and gas-phase thermochemical measurements it has been shown that NEG in the structure of Formula (I) provides high energy HOMO(s) but restores aufbau (i.e. regular) configuration upon protonation to the conjugate acid form. Moreover, it has been shown that the manifestation of this unique electronic structure is not limited to the redox behaviour and that an unprecedented long-range interaction between an unpaired electron and a negative charge results in a dramatic increase in the stability of the radical and acidity of the conjugate acid. The acid-base motif provides an exceptional instrument for switching the orbital configuration, and thus the radical stability, by pH. It is contemplated that such switching has potential applications in biological and industrial fields.

Orbital conversion. To test whether distonic radical anions containing a stabilized radical, non-π-π-conjugated with a negatively charged functional group, display pH-switchable SOMO-HOMO conversion, there was considered two distinct types of stable radical species—cyclic sterically-hindered aminoxyls (as also known as ‘nitroxides’, R₁R₂NO.) and acyclic primary peroxyls (RCH₂OO.). To each species there was introduced a remote carboxylic acid group that was sufficiently separated from the radical centre to eliminate any possibility of π-conjugation, thereby securing only long-range interactions between the investigated radical and donor moieties. It was found that, according to the single- and multireference ab initio and DFT calculations, that the carboxylate anion provides higher-energy doubly-occupied molecular orbitals, and upon its protonation the regular orbital configuration is restored (e.g., see SOMO is HOMO, FIG. 2a-b, and Example 1 to Example 4).

To further confirm of their unusual electronic structure, there was investigated the gas-phase oxidation of the distonic radical anions (see FIG. 2c-d). While COOH-TEMPO (4-carboxy-2,2′,6,6′-tetramethylpiperidine-N-oxyl radical) 5 is easily and reversibly oxidized into a stable closed-shell oxoammonium cation, its SOMO-HOMO converted analogue COO⁻-TEMPO 4 instead favours removal of an electron from its doubly-occupied HOMO resulting in a neutral biradical (triplet ground state, see Example 6), rather than a closed-shell zwitterion (see FIG. 2c). That comparison of the ionization energies (IEs) of the electrons in the SOMO and HOMO is not rigorously representative of the intrinsic energies of these orbitals. If we consider the reverse process—reduction of the neutral triplet—in which an electron is put in the lower of the two singly-occupied orbitals, Coulombic repulsion between the electrons in the resulting doubly-occupied MO would result in these electrons having a lower IE than the unpaired electron. In fact, we were unable to compare the biradical oxidation product of 6 (FIG. 2d) with a corresponding closed-shell zwitterion, because localization of the positive charge on oxygen leads to a strongly disfavoured electronic structure, which upon geometry relaxation undergoes rapid decomposition (see Example 6).

Radical Stability.

Having designed free radicals with pH-controlled SOMO-HOMO energy-level conversion, there was explored their stability in the switched and non-switched forms. A representative practical measure of radical stability is its bond dissociation free energy (BDFE) with simple carbon-centred radicals such as methyl, .CH₃. Therefore calculated were the methyl BDFEs of the carboxy-aminoxyl and peroxyl radicals in their neutral and deprotonated forms in the gas phase at 25° C. and found that deprotonation of the carboxylic group (and thus switching from regular to converted orbital configuration) weakens their bonds with .CH₃. The magnitude of this BDFE-switch, which was defined for a radical COOH—X. as [BDFE(COOH—X—R)-BDFE(COO⁻—X—R)], is ca. 20 kJ mol⁻¹ for the aminoxyl and 15 kJ mol⁻¹ for the peroxyl (or approx. 3.5 and 2.0 orders of magnitude in K_{eq}, see Table 6). Thus, SOMO-HOMO conversion in the deprotonated radicals boosts their stability—an unexpected finding considering the spatial separation and the absence of any obvious π-conjugation interaction between the anionic and radical moieties. In this regard, the calculations confirm that there is no spin density on the remote anion (see Example 2a and Example 2b).

Radical stability is influenced by polar effects, which, at long range, act primarily through-space rather than through-bond. For example, the stability of the aminoxyl radical is affected by resonance between its two forms I and II (see FIG. 3a). It is noted that pH-switchable nitroxide mediated polymerization (NMP) in agent 8 employs polar effects by introducing several basic groups (see FIG. 3b), which upon protonation destabilize H and thus the radical overall. This results in slower decomposition of the corresponding alkoxyamines, indicating an increased strength of their NO—R bonds. However, the magnitude of this pH switch (<15 fold) is significantly smaller than the pH effects observed in the SOMO-HOMO converted species (>2000 fold), despite the fact that the charge in 4 is more distant from the radical centre than in 8 (see FIG. 3b).

Nevertheless, deprotonation of COOH group resulting in an electron-donating carboxylate anion is expected to stabilize II in the trial aminoxyl, and possibly act in a similar way in the peroxyl radical. Therefore, to determine whether this standard polar effect could account for the observed BDFE-switching, there was employed the procedure used to study the effect of amino group protonation on the COOH pkₐ in the homologue series ⁺NR₃(CH₂)ₙCOOH via comparison to a reference system CR₃(CH₂)ₙCOOH that is structurally similar but lacks a polar contribution. There was chosen an alkoxyl radical as the reference system for both the aminoxyl (R₁R₂NO. is substituted by R₁R₂CHO., 9) and the peroxyl (RCH₂O., 11, instead of RCH₂OO.) distonic radical anions and confirmed the absence of the orbital conversion in either COOH protonation state using the same multireference methodology as above (See FIG. 3c and Example 5). Then were calculated the gas-phase BDE-switches (in electronic energy terms to avoid complications from thermal or entropic contributions) for these four homologue series (see FIG. 3d and FIG. 3e, respectively, and Table 7 and Table 8). Although the chosen theoretical methods include implicit dispersion, there have been additionally evaluated its contribution to the BDE-switches using standard dispersion corrections for DFT. In all cases conformations of the (CH₂)ₙ chains were kept anti (extended chains) to eliminate the effects of hyperconjugation and hydrogen bonding, except where such conformations were not local minimum energy structures (see Example 7 and

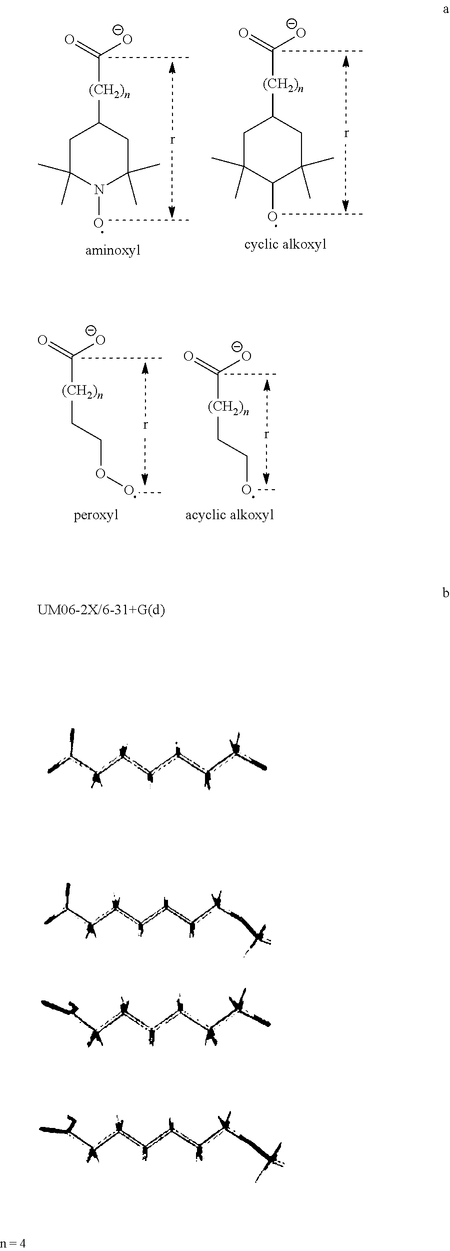

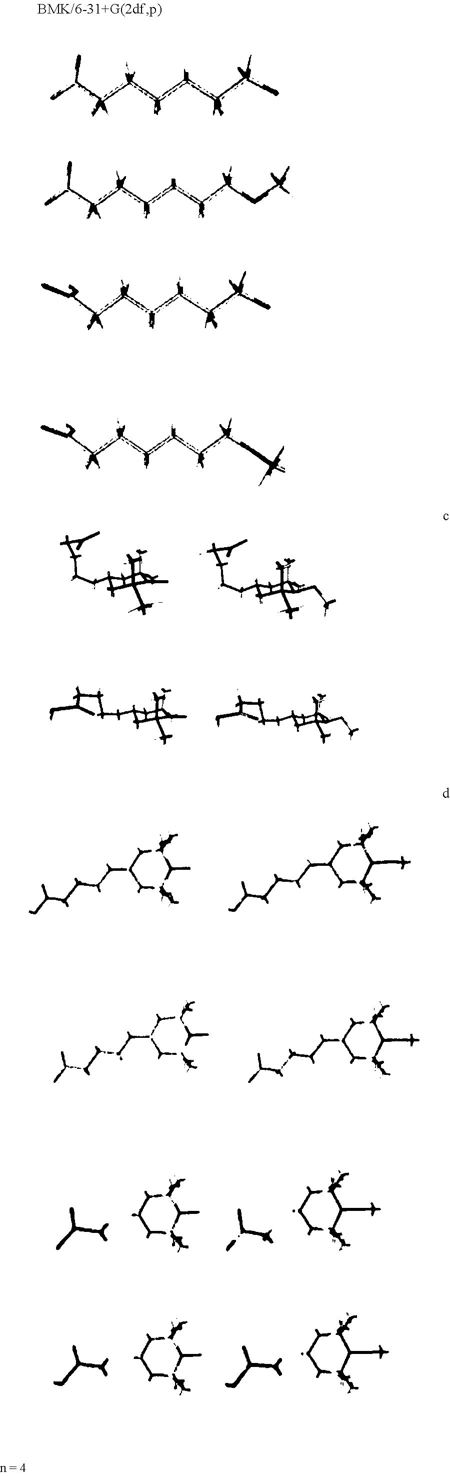

### Example 8).

There was found that all of the radicals exhibit some degree of pH-switching, reflecting a modest polar effect on radical stability, however the switch is significantly larger in the corresponding SOMO-HOMO converted systems (by 10-15 kJ mol⁻¹ at 5 Å separation). In these latter species the BDE-switch at 5 Å separation between the radical and the carboxylate group exceeds 20 kJ ma′, and linearly decays with 1/r, as could indeed be expected for a polar effect. However, the energy breakdown reveals significant contributions of correlation and Hartree components to the magnitude of the BDE-switch, which also depend linearly on 1/r, non-surprisingly considering that the molecular Hamiltonian operator contains 1/r two-electron terms. In the reference non-SOMO-HOMO converted alkoxyl analogues of the aminoxyl radicals, correlation and Hartree components are virtually zero, and the pH-switch arises almost exclusively from the Hartree-Fock exchange, which is similar in magnitude to that in the switched series and also decays as 1/r, thus providing a measure of the polar effect contribution (see Example 3e, right). The situation is somewhat complicated in the case of acyclic alkoxyl series (see Example 3d, right) due to the conformational changes around R₁CH₂—CH₂O. bonds and the related hyperconjugation of the radical centre with the C—H bonds (see Example 7), but it is clear that past 5 Å separation the BDE-switch does not exceed 5 kJ mol⁻¹. There was noted that the dispersion contribution is negligible in all series (see Table 9).

Finally, σ-assistance represents another possible type of through-bond interaction between the remote negative charge and radical centre. To assess its contribution to the pH-switching, we compared carboxy-TEMPO n=4 homologue 15 in the extended-chain conformation to its lowest gas-phase energy conformer 14, in which the anion is closer to the radical moiety (see FIG. 4a). The calculated absolute methyl BDEs of the deprotonated alkoxyamines follow the 1/r dependence (see FIG. 4b). We also constructed a complex of TEMPO and acetic acid 16, structurally resembling the n=4 extended chain conformer 15, but clearly free of any through-bond interaction. Its calculated absolute BDE also sits on the 1/r line (see FIG. 4b), and the BDE-switch is only 1.4 kJ mol⁻¹ lower than that of the corresponding chemically bonded system. These observations confirm the primarily through-space nature of the orbital conversion effect on the radical stability and reveal only a minor contribution from σ-assistance. However, σ-assistance might be contributing to the observed oscillations of the BDE-switch (and in particular its exchange component) in FIG. 3d and affecting the electronic configuration of the biradical oxidation products (see Example 6).

Experimental Verification.

The pH-induced orbital switching affects both the stability of the radical moiety and the acidity of the carboxylic acid group. This is demonstrated by a simple thermocycle that relates the difference in the gas-phase enthalpy of deprotonation (i.e., the gas phase acidities, or GPAs) of carboxy-aminoxyl and its alkoxyamine to the difference in the gas-phase BDEs of the same alkoxyamine in its protonated and deprotonated forms (FIG. 5a and Example 9). Hence the values of the GPA-switches, determined using the kinetic method, represent the direct measures of the BDE-switches that were predicted theoretically. To measure the GPA difference, there was synthesized a dimer complex [A⁻H⁺B⁻] using negative ion electrospray ionization and subjected to collision-induced dissociation in a tandem mass spectrometer. If AH is more acidic than BH, the dissociation equilibrium favours the A⁻+BH product channel, and thus by comparing the abundances of the A⁻and B⁻ product ions there can be determined the GPA-switch for this pair. To assess the BDE-switch in the carboxy aminoxyl, there was prepared a proton-bound dimer where A is that aminoxyl and B is its alkoxyamine. This is illustrated for the carboxy-TEMPO 4 and its methyl alkoxyamine 17 in FIG. 5b, from which it can be seen that 4 is more acidic 17 (positive GPA-switch), in agreement with the calculations (positive BDE-switch).

These experiments for the benzyl and fluoromethyl alkoxyamines of carboxy-TEMPO and carboxy-PROXYL (3-carboxy-2,2′,5,5′-tetramethylpyrrolidine-N-oxyl radical) were repeated, and in all cases the aminoxyl radical is more acidic than the analogous alkoxyamine, which confirms that BDE-switch is due to an effect acting on the radical and is relatively independent on the nature of the leaving R-group. Further, according to the results carboxy-PROXYL is more acidic than carboxy-TEMPO, in agreement with their experimental pKₐ values. There was also studied various combinations of alkoxyamines primarily for benchmarking purposes (see Example 10 and Table 11). There is an excellent agreement between experimental and theoretical results for the full set of dimers (see FIG. 5c), with a mean absolute deviation of only 1.7 kJ mol⁻¹. In order to obtain the absolute GPAs, there was performed similar experiments on the dimers containing the aminoxyl or alkoxyamine of interest and a reference acid with known GPA, benzoic acid. In this manner there was obtained GPAs of 1411 kJ mol⁻¹ for carboxy-TEMPO 4 and 1430 kJ mol⁻¹ for its methyl alkoxyamine 17, which are (i) in exceptional agreement with the theoretical calculations (1414 kJ mol⁻¹ and 1432 kJ mol⁻¹ respectively at 25° C.) and (ii) consistent with the GPA-switch of 13.7 kJ mol⁻¹ determined from direct examination of a dimer formed from this pair (see FIG. 5c).

Due to the transient nature of peroxyl radicals these experiments cannot be applied to verify the calculations on the carboxy peroxyls, because their absolute BDEs are significantly higher and competitive dissociation processes may affect the results. Nevertheless, their increased stability in the deprotonated form was confirmed indirectly, by considering their formation via oxygen addition to the corresponding carbon-centred radicals (that are not stable enough to sustain orbital conversion themselves). According to the calculations, addition of oxygen to the ⁻OOC—(CH₂)₃CH₂. radical is more exoergic (by 20 kJ mol⁻¹ in the gas phase at 25° C.) than to HOOC—(CH₂)₃CH₂—, because as shown above distonic peroxyl radical anion is stabilized by the SOMO-HOMO conversion. Experimentally rate constants of oxygen addition to substrates of a similar chemical nature are equal to 1.8×10⁻¹⁰ cm³ mol⁻¹s⁻¹ (representing a reaction efficiency of 30% at 34° C.) for 4-carboxycyclohexyl and 1.3×10⁻¹¹ cm³ mol⁻¹ s⁻¹ (or 3% at 25° C.) for regular cyclohexyl radicals (see Examples). Considering that oxygen addition is a fast process with an early transition state, this about 15-fold difference in reactivity indicates substantial stabilization of the anionic (versus neutral) radical product.

The GPA experiments indicate that the effect of the R-group on this BDE-switch is mild (see Example 4c), further confirming that it originates primarily as an effect on the stability of X. (rather than X—R). There was also explored the effect of other anionic groups, such as thiocarboxylate, phosphate, sulfate and alkoxide, using TEMPO 13 as a (substrate (18-21 in FIG. 6a). In all cases there is a significant BDE switch, and hence orbital conversion is not limited to only the carboxylate donor unit.

SOMO-HOMO conversion was also investigated in the deprotonated nucleic acid radicals, say for example formed as a result of oxidative damage via hydrogen atom abstraction by, for example, hydroxyl radical.

While abstraction from the sugar moiety yields relatively unstable carbon-centred radicals, not expected to undergo orbital rearrangement, abstraction from the base leads to stable delocalized radical with a potential for SOMO-HOMO conversion (FIG. 6b).

According to the calculated BDEs, the calculations show that both the model DNA 22 and RNA 23 radicals—abstraction from the 4^{th} position of the sugar ring is not affected by the protonation state of the phosphate groups, whereas deprotonation increases the vulnerability of the base moiety by over 35 kJ mol⁻¹. Furthermore, upon vertical ionization regular sugar-derived radicals form closed-shell zwitterions (i.e. from removal of an electron from the singly-occupied molecular orbital, i.e. SOMO=HOMO), whereas converted base-derived radicals favour biradical species (i.e. removal of an electron from a doubly occupied molecular orbital, i.e. the SOMO≠HOMO), similarly to the carboxy-aminoxyl radicals (see Example 12). Indeed, the radicals formed by H-abstraction from the base nitrogen atoms are among the major detected products in the model experimental and computational studies.

### Examples and Methods

### Theoretical Procedures.

All ab initio and density functional theory calculations were performed using Gaussian 09, QChem 3.2, Molpro 2009.1 and GAMESS 2010 software packages. Geometries of all species, with the exception of the products of vertical oxidation, were fully optimized using M06-2X/6-31+G(d) and, selectively, BMK/6-31+G(2df,p) methods, and the frequencies were calculated at the same levels and scaled by recommended scale factors. For the homologue series, (CH₂)ₙ chains were kept in the extended conformations, while full conformational searches at a resolution of 60° were performed for all other species in the present work. Accurate electronic energies were calculated using a variety of methods ranging from DFT and MP2 to high-level composite G3(MP2)RAD, G3(MP2,CC) and G4(MP2)-6X procedures. Calculations on radicals were performed with an unrestricted wave function except in cases designated with an “R” prefix where a restricted open-shell wave function was used. For large systems (over 15-20 heavy atoms), where high-level composite calculations were not feasible computationally, double-layer ONIOM-type procedure was applied instead and the MP2/6-311+G(3df,2p) method was used for the full system. Reported thermochemical values (gas-phase bond dissociation energies, acidities and relative ionisation energies) were calculated in conjunction with the gas-phase entropies and thermal corrections at 25° C., obtained using standard textbook formulas for the statistical thermodynamics of an ideal gas under the harmonic oscillator approximation in conjunction with the optimized geometries and scaled frequencies. Described methodologies have been extensively tested against experimental data and shown to deliver results to within the chemical accuracy (ca. 5 kJ mol⁻¹ for bond dissociation energies and 0.050 V for redox potentials). It should be emphasized that in the majority of cases studied here both the absolute values of the aforementioned thermochemical parameters and, more importantly, their relative differences (e.g., BDE- and GPA-switches) vary within only a small range of 7 kJ mol⁻¹ depending on the theoretical method without affecting the reactivity trends and the conclusions.

Ordering and occupation of molecular orbitals of the various radical species were evaluated at a range of theoretical levels, including single-reference HF, DFT and MP2 calculations with different basis sets, as well as multireference MCSCF and MRPT2 simulations (as implemented in GAMESS) using 6-31+G(d) basis on a (9,5) active space constructed of occupied orbitals only, and CASSCF/STO-3G method (as implemented in Gaussian 09) on a (7,8) active space containing both occupied and virtual molecular orbitals. The adopted approaches were found to accurately describe the orbital configuration in several earlier studies of different SOMO-HOMO converted radicals. It was found that orbital arrangements obtained using single-reference methods are strongly dependent on the method, basis set, type of wave function and even software code. Nonetheless, the two conceptually different multireference approaches that were used both confirm the predicted SOMO-HOMO energy-level conversion in the trial carboxy aminoxyl and peroxyl radicals, as well as its absence in their protonated forms and in the reference alkoxyl species. The detailed description of the theoretical procedures and expanded set of results are given in the examples.

Experimental Procedures.

Alkoxyamines were synthesized from aminoxyl radical precursors by a standard literature procedure. Equimolar mixtures of aminoxyl radical and alkoxyamine were prepared pairwise in methanol to a final concentration of ca. 10 μM. Proton-bound dimers were generated by negative ion electrospray ionization upon infusion of these methanolic solutions at a rate of 5 μL min⁻¹ into the ion source of a Waters QuattroMicro (Manchester, UK) triple quadrupole mass spectrometer. The mass spectrometer was operated in the negative ion mode, and controlled by Micromass MassLynx software (Version 4.1). The capillary voltage was set to −3.0 kV, cone voltage −20 V, source temperature 80° C. and desolvation temperature 110° C., to optimize the production of desired proton-bound cluster ions. Nitrogen was used as the drying gas, at a flow rate of 300 L h⁻¹ while collision-induced dissociation (CID) experiments used laboratory frame energies of 5-25 V as required and argon as the collision gas (3.0±0.1 mTorr). All mass spectra and ion abundance ratios reported here are the averages of at least 200 cumulative scans. The kinetic method was used for the determination of relative and absolute gas phase deprotonation enthalpies based on the relative ion abundances resulting from competitive dissociation of a mass-selected cluster ion.

### Orbital Configurations

### Methodology.

In order to determine the orbital configurations of the trial carboxy-aminoxyl and -peroxyl distonic radical anions, first used was unrestricted single-reference ab initio and DFT methods. Specifically, there was matched the alpha and beta-spin electrons by both their energies and shapes: whenever there is an occupied beta orbital that matches an occupied alpha orbital, they form a doubly-occupied molecular orbital, whereas one occupied alpha orbital that matches a low-lying unoccupied beta orbital corresponds to the radical's SOMO. This procedure performed using UHF and UMP2 wave function-based methods, as well as UM06-2X density functional theory, with basis sets ranging from STO-3G to 6-311+G(3df,2p) in conjunction with the geometries optimized at the same theoretical level. In all cases there was observed conversion of the orbital configuration resulting in a number of doubly occupied molecular orbitals having higher energy than the singly-occupied one, and selected examples are given in Example 1. Spin densities were plotted using GaussView 5.0 and orbital plots were built using IQmol 2.1 visualization packages.

Furthermore, it was confirmed that this orbital rearrangement in the trial species using two different multireference methods. Firstly, there was applied the MCSCF method in conjunction with the 6-31+G(d) basis set using GAMESS software package. The active space was chosen to include the 5 highest occupied orbitals containing 9 electrons in order to model the possible configurations in the ground rather than in the excited states. These orbitals were chosen to represent the unpaired electron, the oxygen lone pairs on the radical moiety and the three doubly occupied orbitals corresponding to the three-centred π-bond of the carboxylate ion and the lone pairs on its two oxygen atoms. These MCSCF simulations were followed by the MRPT2 calculations on the same active space. MCSCF orbital plots were built using MacMolPlt v.7.4.2; optimized MCSCF and MRPT2 configurations for the two trial species are shown in Example 2. Secondly, there was characterized the orbital configurations in the trial compounds using somewhat different complete active space methodology, CASSCF(7,8)/STO-3G as implemented in Gaussian 09. In this case the active space included four occupied orbitals (SOMO and three orbitals of the carboxylate, described above) and four corresponding virtual orbitals; orbitals were built using GaussView 5.0. Obtained results again confirmed converted configuration to be the dominant one for the trial compounds (see Example 3).

There was then applied the same multireference procedures to characterize the orbital space in the protonated analogues of the trial carboxy-peroxyl and aminoxyl radicals (Example 4) and in the protonated and deprotonated forms of the reference alkoxyl radicals (orbital shapes are principally similar to those in Example 2 to Example 4, therefore shown are only the eigenvalues of different configurations, Example 5). The active space was constructed in a similar way as above, except that for the protonated species one of the oxygen lone-pair orbitals on the carboxylic moiety involved instead the O—H σ-bonding orbital.

### Example 1

### Single-Reference Molecular Orbitals of the Trial Distonic Radical Anions

UHF/STO-3G (left) and UM06-2X/6-31+G(d) (right) spin densities (transparent surfaces) and alpha and beta molecular orbitals (solid surfaces) of the trial carboxy-peroxyl (top) and aminoxyl (bottom) radicals. Numbers in bold are orbital numbers (in the order of increasing energy) and numbers in italics are the orbital energies. Numbers corresponding to the singly-occupied molecular orbitals are underlined.

| | |
|---|---|
| UHF/STO-3G | UM06-2X/6-31+G(d) |

| | | |
|---|---|---|
| 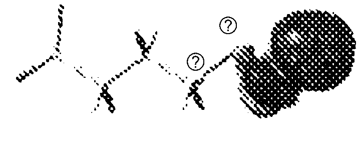 | 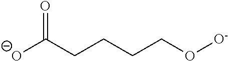 | 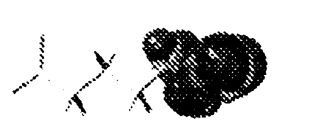 |

| alpha | | | beta | | | alpha | | | beta | | |
|---|---|---|---|---|---|---|---|---|---|---|---|
| | | | 36 | [symbol] | 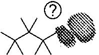 | | | | 36 | 0.024 | 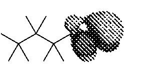 |
| 0.055 | 36 | 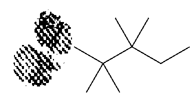 | 35 | [symbol] | 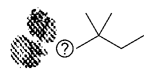 | −0.119 | 36 | 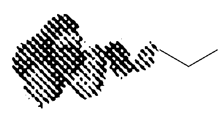 | 35 | [symbol] | 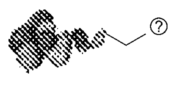 |
| 0.019 | 35 | 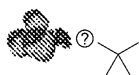 | 34 | [symbol] | 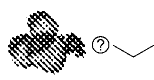 | −0.124 | 35 | 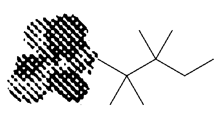 | 34 | [symbol] | 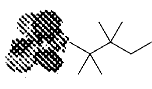 |
| 0.004 | 34 | 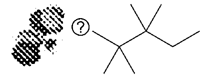 | 32 | [symbol] | 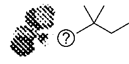 | −0.127 | 34 | 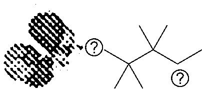 | 33 | [symbol] | 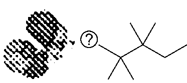 |
| [symbol] | 30 | | | | | [symbol] | 31 | 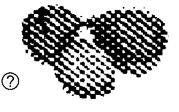 | | | |
| 0.273 | 29 | 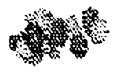 | 29 | [symbol] | 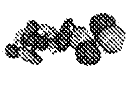 | −0.280 | 30 | 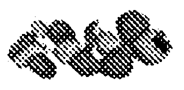 | 30 | [symbol] | 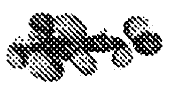 |

| | | |
|---|---|---|
| 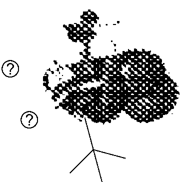 | 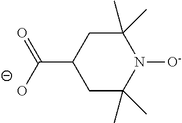 | 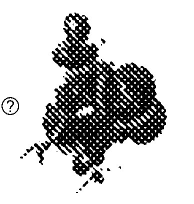 |

| alpha | | | beta | | | alpha | | | beta | | |
|---|---|---|---|---|---|---|---|---|---|---|---|
| | | | [symbol] | [symbol] | 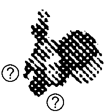 | | | | 55 | 0.097 | 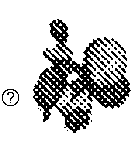 |
| [symbol] | 55 | 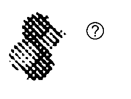 | 54 | [symbol] | 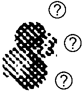 | [symbol] | 55 | 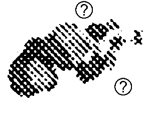 | 54 | [symbol] | 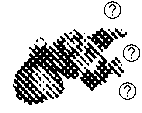 |
| 0.010 | 54 | 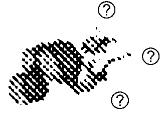 | 53 | [symbol] | 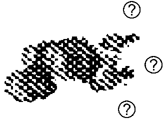 | −0.133 | 54 | 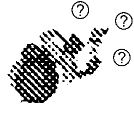 | 53 | [symbol] | 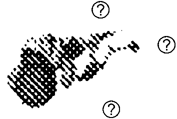 |
| −0.007 | 53 | 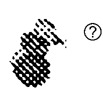 | 52 | [symbol] | 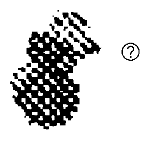 | [symbol] | 53 | 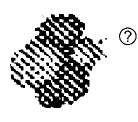 | 52 | [symbol] | 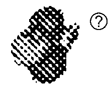 |
| [symbol] | 52 | 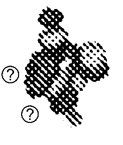 | | | | [symbol] | 52 | 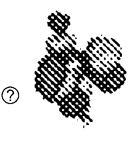 | | | |
| [symbol] | 51 | 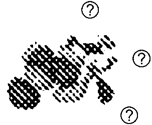 | 51 | [symbol] | 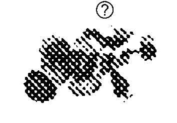 | −0.220 | 51 | 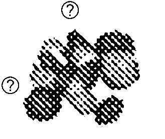 | 51 | [symbol] | 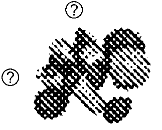 |

[symbol]

indicates data missing or illegible when filed

### Example 2

### MCSCF and MRPT2 Molecular Orbitals of the Trial Distonic Radical Anions

Plots of MCSCF(9,5)/6-31+G(d) optimized molecular orbitals and MCSCF(9,5)/6-31+G(d) and MRPT2(9,5)/6-31+G(d) optimized orbital configurations for c the trial carboxy-peroxyl (left) and -aminoxyl (right) radicals.

| | | |
|---|---|---|
| 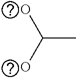 | 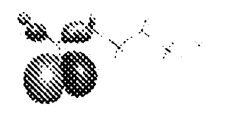 | MCSCF(9,5) ? |
| | 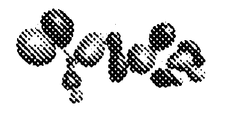 | |
| 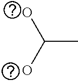 | 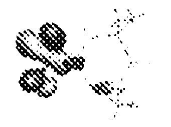 | MCSCF(9,5) ? |
| | 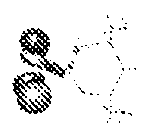 | |

| | |
|---|---|
| 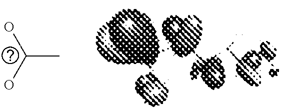 | MRPT2(9,5) ? |
| 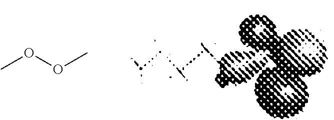 | |
| 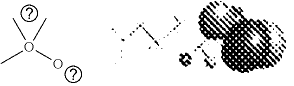 | |
| 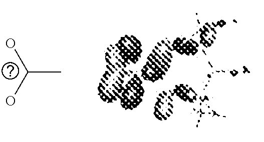 | MRPT2(9,5) ? |
| 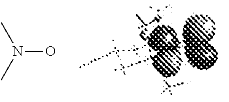 | |
| 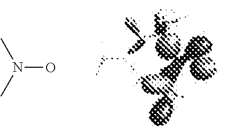 | |

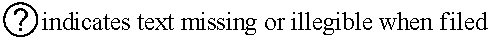

### Example 3

### CASSCF Molecular Orbitals of the Trial Distonic Radical Anions

CASSCF(7,8)/STO-3G optimized molecular orbitals and the main configurations with their eigenvalues shown in bold font for the trial carboxy peroxyl (left) and aminoxyl (right) radicals.

| occupied | virtual | 0.96 −0.20 −0.06 −0.06 |
|---|---|---|
| 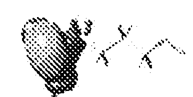 | 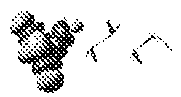 | 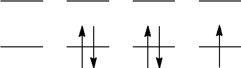 |
| 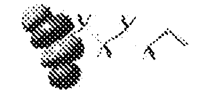 | 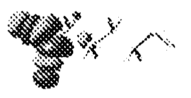 | 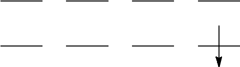 |
| 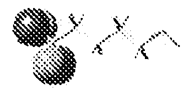 | 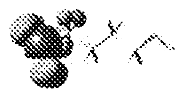 | 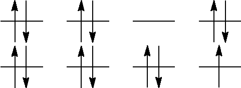 |
| 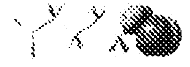 | 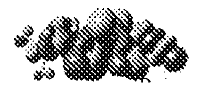 | 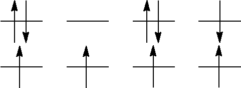 |
| 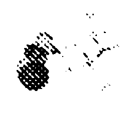 | 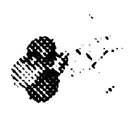 | 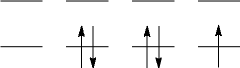 |
| 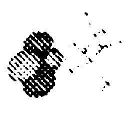 | 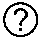 | 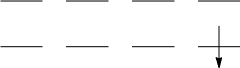 |
| 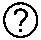 | 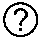 | 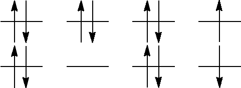 |
| 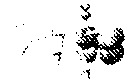 | 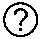 | 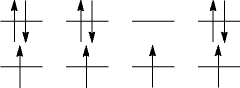 |

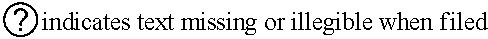

### Example 4

### Multireference Molecular Orbitals of the Trial Protonated Radicals

Plots of the optimized MCSCF(9.5)/6-31+G(d) molecular orbitals. MCSCF(9,5)/6-31+G(d) and MRPT2(9,5)/6-31+G(d) orbital configurations, as well as CASSCF(7,8)/STO-3G optimized molecular orbital plots and the main configurations with their eigenvalues shown in bold font for the protonated carboxy peroxyl (top) and -aminoxyl (bottom) radicals. Orbitals were built using MacMolPlt v.7.4.2 and GaussView 5.0 packages.

| 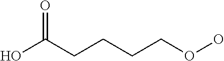 | | |
|---|---|---|
| 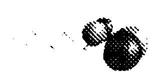 | MCSCF(9,5) ? | |
| 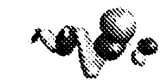 | | |
| 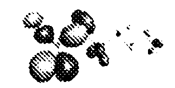 | | |
| 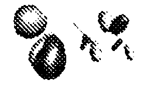 | MRPT2(9,5) ? | |
| 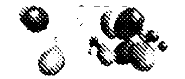 | | |
| 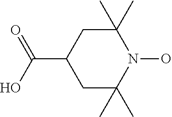 | | |
| 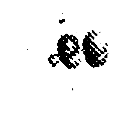 | MCSCF(9,5) ? | |
| 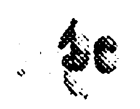 | | |
| 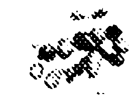 | | |
| 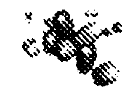 | MRPT2(9,5) ? | |
| 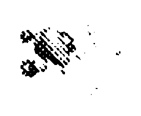 | | |
| occupied | virtual | CASSCF(7,8) |
| | | 0.96 0.23 0.10 0.07 |
| 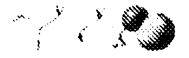 | 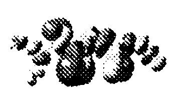 | 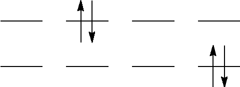 |
| 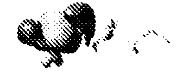 | 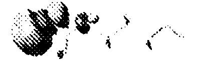 | 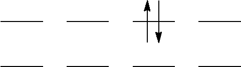 |
| 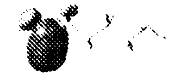 | 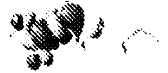 | 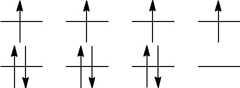 |
| 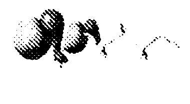 | 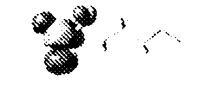 | 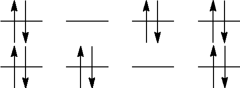 |
| | | 0.97 0.20 −0.05 −0.05 |
| 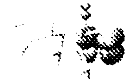 | 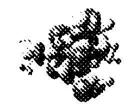 | 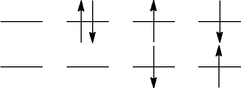 |
| 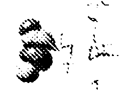 | 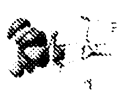 | 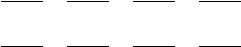 |
| 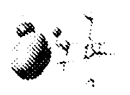 | 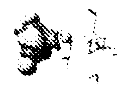 | 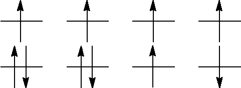 |
| 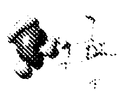 | 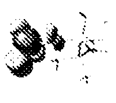 | 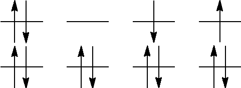 |

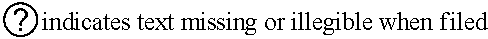

### Example 5

### Multireference Molecular Orbitals of the Reference Carboxy-Alkoxyl Radicals

MCSCF(9,5)/6-31+(G(d) and MRPT2(9,5)/6-31+G(d) optimized orbital configurations, as well as CASSCF(7.8)/STO-3G dominant optimized configurations with their eigenvalues shown in bold font for the reference alkoxyl radicals in both protonation states of the carboxylic group

| | CASSCF(7,8) |
|---|---|
| 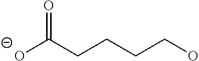 | |
| MCSCF(9,5) | 0.96 0.20 0.06 0.06 |
| ALPHA \|BETA \|COEFFICIENT 11111 \| 11110 \| 0.9984984 11111 \| 11101 \| 0.0523857 11111 \| 11011 \| 0.0133048 11111 \| 01111 \| 0.0074483 11111 \| 10111 \| 0.0049225 | 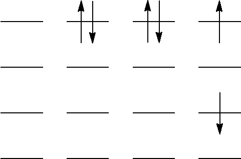 |
| MRPT2(9,5) | |
| ALPHA \|BETA \|COEFFICIENT 11111 \| 11110 \| 0.9999849 11111 \| 11101 \| −0.0048994 11111 \| 11011 \| 0.0024784 11111 \| 01111 \| 0.0001972 11111 \| 10111 \| 0.0001689 | 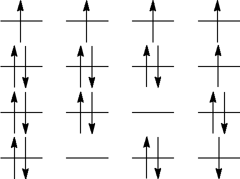 |
| 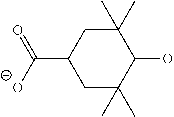 | |
| MCSCF(9,5) | 0.96 0.20 0.05 0.06 |
| ALPHA \|BETA \|COEFFICIENT 11111 \| 11110 \| 0.9999989 11111 \| 11101 \| 0.0012841 11111 \| 11011 \| 0.0007842 11111 \| 01111 \| 0.0001157 11111 \| 10111 \| 0.0000224 | 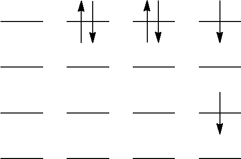 |
| MRPT2(9,5) | |
| ALPHA \|BETA \|COEFFICIENT 11111 \| 11110 \| 0.9994741 11111 \| 11101 \| 0.0285471 11111 \| 11011 \| 0.0139682 11111 \| 01111 \| 0.0054626 11111 \| 10111 \| 0.0033977 | 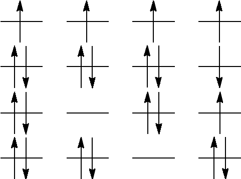 |
| 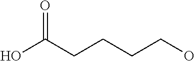 | |
| MCSCF(9,5) | 0.97 0.20 0.05 0.05 |
| ALPHA \|BETA \|COEFFICIENT 11111 \| 11110 \| 0.9967762 11111 \| 11101 \| 0.0653481 11111 \| 11011 \| 0.0401235 11111 \| 01111 \| 0.0223079 11111 \| 10111 \| 0.0077001 | 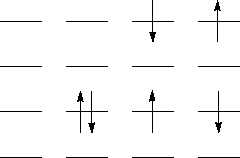 |
| MRPT2(9,5) | |
| ALPHA \|BETA \|COEFFICIENT 11111 \| 11110 \| 0.9999998 11111 \| 11101 \| 0.0004980 11111 \| 11011 \| 0.0002354 11111 \| 01111 \| −0.0000601 11111 \| 10111 \| 0.0000503 | 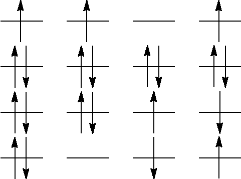 |
| 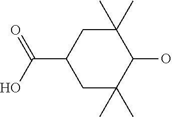 | |
| MCSCF(9,5) | 0.96 0.23 0.10 0.07 |
| ALPHA \|BETA \|COEFFICIENT 11111 \| 11110 \| 0.9641431 11111 \| 11101 \| −0.2640581 11111 \| 11011 \| 0.0236074 11111 \| 01111 \| 0.0109787 11111 \| 10111 \| 0.0048577 | 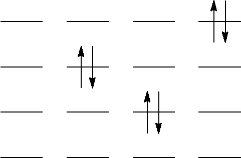 |
| MRPT2(9,5) | |
| ALPHA \|BETA \|COEFFICIENT 11111 \| 11110 \| 0.9980628 11111 \| 11101 \| 0.0621265 11111 \| 11011 \| 0.0073708 11111 \| 01111 \| 0.0021107 11111 \| 10111 \| −0.0009288 | 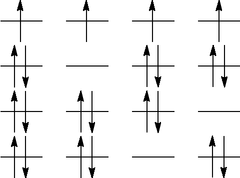 |

### Oxidation (Ionization) of the Distonic Radical Anions

### Methodology.

As an additional test of the unusual orbital configurations of the trial species there was investigated their ionization (one-electron oxidation). For both substrates we found that the biradical product had a triplet ground state (see Example 6). Calculations on the singlet biradicals were performed using the broken symmetry DFT approach in conjunction with the optimized triplet biradical geometries. In the case of the deprotonated carboxy-TEMPO it was possible to successfully model both the vertical and adiabatic oxidation products, characterized with stable wave functions and equilibrium geometries. Calculated atomic charges and spin densities (see FIG. 2) as well as the S² values confirm that located structures are true closed-shell zwitterions and open-shell biradicals. Both species are stable upon geometry relaxation, which in the case of the zwitterion is accompanied by the planarization of the nitrogen. It was found that independent of the computational methodology the triplet biradical is more stable (by approximately 10-25 kJ mol⁻¹) than the zwitterion, which is consistent with the orbital conversion in the parent carboxy aminoxyl. However, oxidation of the trial carboxy peroxyl to a zwitterion cannot be achieved due to extreme instability of such product involving a localized positive charge on an oxygen atom. Upon geometry relaxation it releases CO₂ and undergoes intramolecular cyclization, therefore it is not possible to use it in the diagnostics of the orbital configuration in the parent carboxy peroxyl. Nevertheless, the triplet biradical product is stable both in the frozen and relaxed geometries (see Example 6).

### Example 6

### Properties of the One-Electron Oxidation Open- and Closed-Shell Products

Competitive pathways of one-electron oxidation (ionization) of the SOMO-HOMO energy-level converted distonic anion radicals into closed-shell (singlet) zwitterions and open-shell (triplet and singlet) biradicals. For the oxidation products, shown are their optimized UM06-2X/6-31+G(d) geometries. Numbers next to structures are UM06-2X/6-31+G(d) and UMP2/GTMP2Large (in brackets) Mulliken atomic spin densities and charges (italics). Spin contamination in the open-shell species is reflected in their S² (UM06-2X/6-31+G(d) and in brackets UMP2/GTMP2Large) and T1 diagnostics (UCCSD(T)/6-31+G(d)) values. Relative electronic energies of the oxidation products (kJ mol⁻¹) are calculated using UM06-2X/6-31+G(d) and modified G3(MP2,CC)(+) methods

| | |
|---|---|
| Closed-shell | Open-shell |

| | |
|---|---|
| Triplet | Singlet |

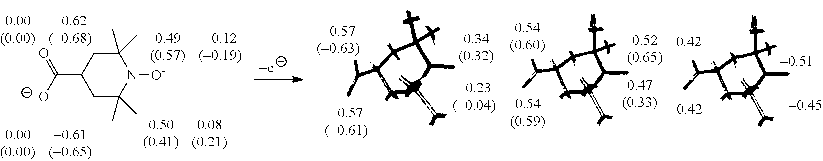

| | | | | |
|---|---|---|---|---|
| | | | Vertical | |
| S² = 0.75 (0.77), T1 = 0.015 | Spin contamination | S² = 0.00 (0.00), T1 = 0.018 | S² = 2.02 (2.00), T1 = 0.021 | |
| | UM06-2X/6-31+G(d) | 23.08 | 0.00 | |
| | G3(MP2.CC)(+) | 19.93 | 0.00 | |
| | | | Adiabatic | |
| | Spin contamination | S² = 0.00 (0.00), T1 = 0.016 | S² = 2.01 (2.06), T1 = 0.018 | S² = 1.01 |
| | UM06-2X/6-31+G(d) | 22.28 | 0.00 | 149.25 |
| | G3(MP2.CC(+) | 10.83 | 0.00 | |

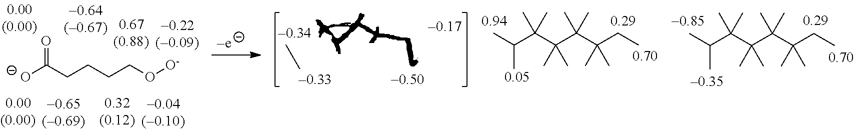

| | | | |
|---|---|---|---|
| S² = 0.75 (0.77), T1 = 0.021 | Spin contamination | S² = 2.01, T1 = 0.030 | S² = 1.032 |
| | UM06-2X/6-31+G(d) | 0.00 | 52.08 |

### Bond Dissociation Energies

### Methodology.

To obtain accurate energetics of the bonds the trial radicals form with the methyl, there was used several high-level composite methods. Firstly, there was applied the G3(MP2)-RAD and G3(MP2,CC) methods with several modifications. Specifically, accurate description of the anionic species requires introduction of the diffuse functions into the basis set, therefore in all calculations there was used 6-31+G(d) basis instead of the default 6-31 G(d) basis, which are denoted as the G3(MP2)-RAD(+) and G3(MP2,CC)(+) procedures. There was also chosen to use the M06-2X density functional instead of the B3LYP functional. Albeit being one of the most commonly used methods, B3LYP is known to suffer from a number of serious drawbacks, while a new family of Minnesota functionals exhibit superior performance, in particular for the thermochemistry of radical reactions, which is supported by the results of this work (see below). Furthermore, standard G3(MP2) methods approximate CCSD(T) calculations with a large triple-ζ basis from calculations with a double-basis set via basis set corrections carried out at the R(O)MP2 (in G3(MP2)-RAD) and UMP2 (in G3(MP2,CC) levels. In both procedures there was used URCCSD(T) energies, calculated using the Molpro code, and there was also used high level correction (HLC) values of the G3(MP2)-RAD method in the G3(MP2,CC) calculations, as the former method has been parameterized specifically for the radical reactions. Secondly, there was used a recent G4(MP2)-6X method, the main differences of which from the G3-family are 1) BMK/6-31+G(2df,p) method is used for geometries and frequencies and 2) the estimated Hartree-Fock-limit energy extrapolated to the complete basis set limit is the basic component of the composite energy. This method is more expensive computationally than the G3(MP2) methods and thus was feasible only for the smaller peroxyl species, but not the aminoxyls. Finally, there was complemented accurate electronic energies with the entropies and thermal corrections, calculated under the harmonic oscillator approximation in conjunction with the optimized geometries and scaled frequencies, the results for the absolute bond dissociation energies and their differences (BDE-switches, as defined previously) for the two trial species—carboxy peroxyl and aminoxyl in protonated and deprotonated forms—are collected in Table 6. The G3(MP2)-RAD(+) and G3(MP2,CC)(+) results differ insignificantly (within 4 kJ mol⁻¹), and there is a slightly greater variation (ca. 7-8 kJ mol⁻¹) from the G4 method in the absolute values. Importantly, an excellent agreement within less than 1 kJ mol⁻¹ variation is obtained with all the methods for the values of the BDE-switches.

TABLE 1
Calculated bond dissociation energies and BDE-switches (in electronic energy
terms in the gas phase using UM06-2X/6-31+ G(d) level of theory, kJ mol⁻¹); ^{a}Shown are
the closed-shell deprotonated compounds; ^{b}In deprotonated form, as shown in the first
column; ^{c}In fully-protonated form; ^{d}Intramolecular electron transfer occurs in the open-
shell species.

| Species^{a} | BDE (-)^{b} | BDE (H)^{c} | switch |
|---|---|---|---|
| 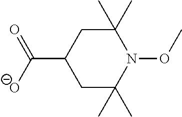 | 214.162 | 233.184 | 19.022 |
| 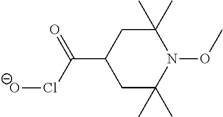 | 215.474 | 234.819 | 19.345 |
| 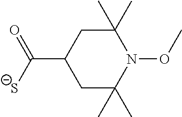 | 216.107 | 233.688 | 17.581 |
| 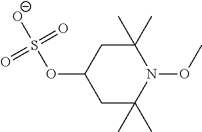 [SMILES: CON1C(C)(C)CC(OS(=O)(=O)[O-])CC1(C)C] | 218.135 | 235.127 | 16.991 |
| 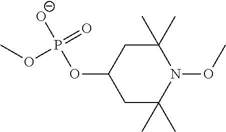 | 217.532 | 232.930 | 15.398 |
| 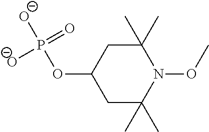 [SMILES: CON1C(C)(C)CC(OP(=O)([O-])[O-])CC1(C)C] | 202.059 | 232.930 | 30.871 |
| 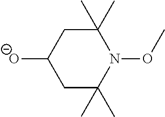 | 209.120 | 232.834 | 23.714 |
| 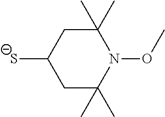 | 212.943 | 231.201 | 18.258 |
| 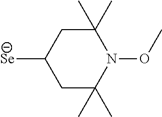 | 376.237^{d} | 233.482 | −142.755 |
| 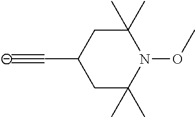 | 215.573 | 232.799 | 17.226 |
| 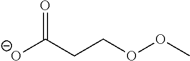 | 283.991 | 307.318 | 23.326 |
| 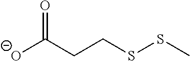 | 252.444 | 269.360 | 16.916 |
| 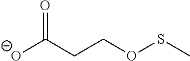 | 280.130 | 297.071 | 16.941 |
| 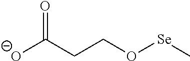 | 252.219 | 263.599 | 11.380 |
| 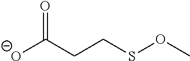 | 243.166 | 261.708 | 18.542 |
| 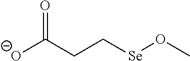 | 273.387 | 288.271 | 14.884 |
| 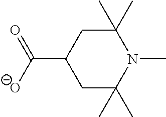 | 332.274 | 342.160 | 9.886 |
| 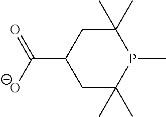 | 306.975 | 311.645 | 4.670 |
| 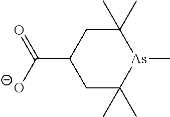 | 260.681 | 263.922 | 3.242 |
| 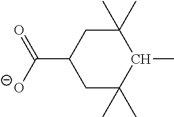 | 350.142 | 350.758 | 0.616 |
| 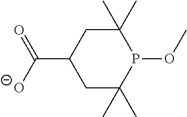 | 242.468 | 265.548 | 23.080 |
| 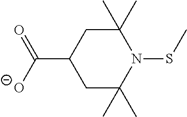 | 233.193 | 250.422 | 17.229 |
| 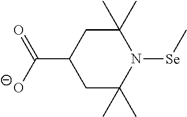 | 222.089 | 236.196 | 14.107 |
| 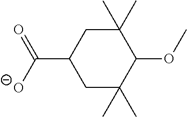 | 362.441 | 372.361 | 9.920 |
| 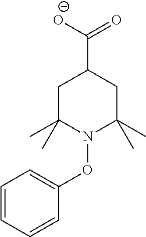 [SMILES: CC1(C)CC(C(=O)[O-])CC(C)(C)N1Oc1ccccc1] | 173.194 | 187.055 | 13.861 |
| 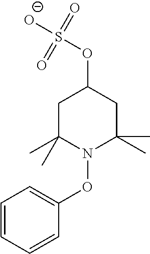 [SMILES: CC1(C)CC(OS(=O)(=O)[O-])CC(C)(C)N1Oc1ccccc1] | 175.793 | 187.874 | 12.081 |
| 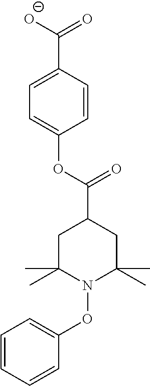 [SMILES: CC1(C)CC(C(=O)Oc2ccc(C(=O)[O-])cc2)CC(C)(C)N1Oc1ccccc1] | 181.457 | 187.409 | 5.952 |
| 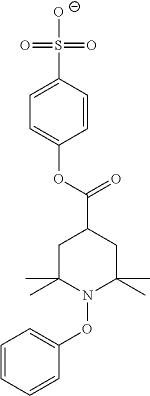 [SMILES: CC1(C)CC(C(=O)Oc2ccc(S(=O)(=O)[O-])cc2)CC(C)(C)N1Oc1ccccc1] | 182.058 | 188.380 | 6.322 |
| 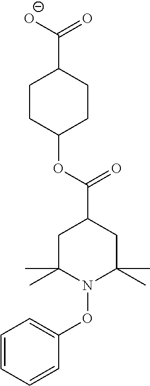 [SMILES: CC1(C)CC(C(=O)OC2CCC(C(=O)[O-])CC2)CC(C)(C)N1Oc1ccccc1] | 179.614 | 186.053 | 6.439 |
| 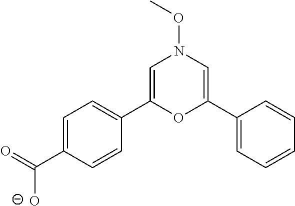 [SMILES: CON1C=C(c2ccccc2)OC(c2ccc(C(=O)[O-])cc2)=C1] | 213.126 | 230.522 | 17.396 |
| 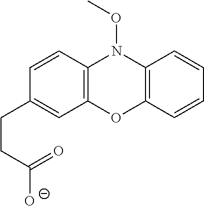 [SMILES: CON1c2ccccc2Oc2cc(CCC(=O)[O-])ccc21] | 208.510 | 221.628 | 13.118 |
| 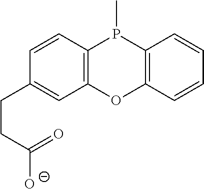 [SMILES: CP1c2ccccc2Oc2cc(CCC(=O)[O-])ccc21] | 279.680 | 284.445 | 4.764 |
| 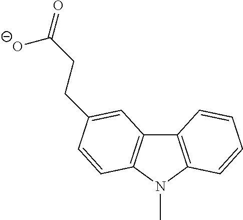 [SMILES: Cn1c2ccccc2c2cc(CCC(=O)[O-])ccc21] | 335.375 | 361.210 | 25.835 |
| 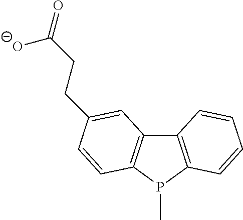 [SMILES: Cp1c2ccccc2c2cc(CCC(=O)[O-])ccc21] | 285.717 | 293.600 | 7.883 |
| 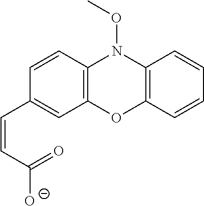 [SMILES: CON1c2ccccc2Oc2cc(/C=C\C(=O)[O-])ccc21] | 205.565 | 223.936 | 18.372 |
| 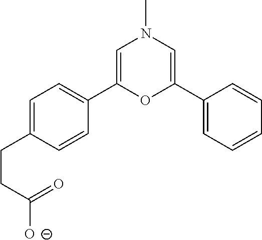 [SMILES: CN1C=C(c2ccccc2)OC(c2ccc(CCC(=O)[O-])cc2)=C1] | 217.330 | 227.870 | 10.540 |
| 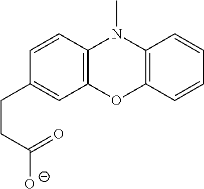 [SMILES: CN1c2ccccc2Oc2cc(CCC(=O)[O-])ccc21] | 255.404 | 264.094 | 8.689 |
| 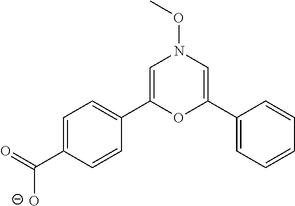 [SMILES: CON1C=C(c2ccccc2)OC(c2ccc(C(=O)[O-])cc2)=C1] | 199.865 | 215.200 | 15.335 |
| 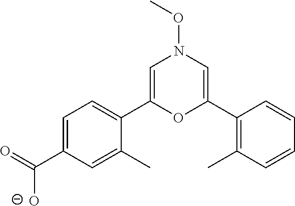 [SMILES: CON1C=C(c2ccccc2C)OC(c2ccc(C(=O)[O-])cc2C)=C1] | 204.298 | 219.262 | 14.964 |
| 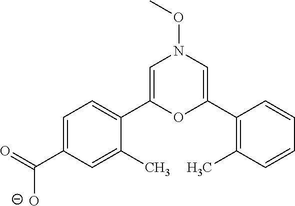 [SMILES: CON1C=C(c2ccccc2C)OC(c2ccc(C(=O)[O-])cc2C)=C1] | 208.370 | 221.600 | 13.230 |
| 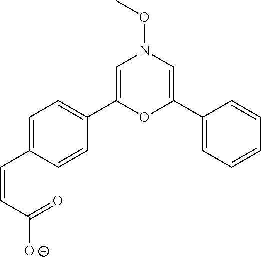 [SMILES: CON1C=C(c2ccccc2)OC(c2ccc(/C=C\C(=O)[O-])cc2)=C1] | 202.890 | 214.430 | 11.540 |
| 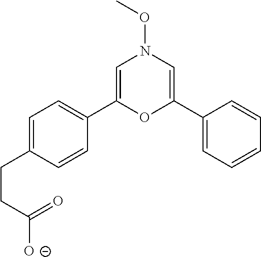 [SMILES: CON1C=C(c2ccccc2)OC(c2ccc(CCC(=O)[O-])cc2)=C1] | 203.516 | 213.905 | 10.389 |
| 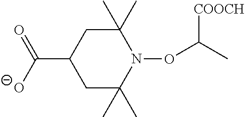 | 175.739 | 189.335 | 13.6 |
| 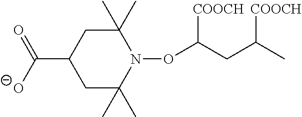 | 179.888 | 191.677 | 11.8 |
| 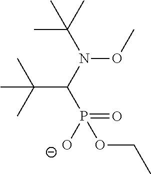 [SMILES: CCOP(=O)([O-])C(N(OC)C(C)(C)C)C(C)(C)C] | 190.427 | 211.071 | 20.644 |
| 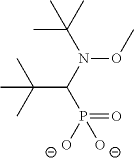 [SMILES: CON(C(C(C)(C)C)P(=O)([O-])[O-])C(C)(C)C] | 160.802 | 211.071 | 50.269 |
| 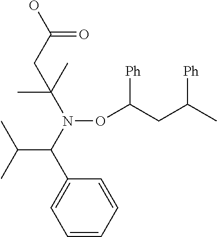 [SMILES: CC(CC(ON(C(c1ccccc1)C(C)C)C(C)(C)CC(=O)O)c1ccccc1)c1ccccc1] | 163.551 | 190.336 | 26.785 |
| 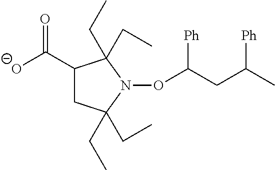 [SMILES: CCC1(CC)CC(C(=O)[O-])C(CC)(CC)N1OC(CC(C)c1ccccc1)c1ccccc1] | 161.971 | 184.127 | 22.155 |
| 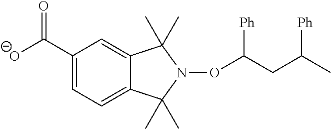 [SMILES: CC(CC(ON1C(C)(C)c2ccc(C(=O)[O-])cc2C1(C)C)c1ccccc1)c1ccccc1] | 173.829 | 191.979 | 18.149 |
| 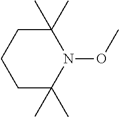 | 231.175 | | N/A |
| 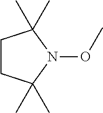 | 218.165 | | N/A |
| 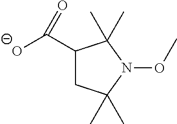 | 200.213 | 221.293 | 21.081 |
| 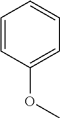 | 313.709 | | N/A |
| 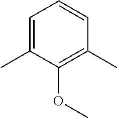 | 283.247 | | N/A |
| 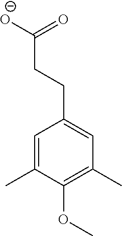 | 278.208 | 253.381 | 24.828 |
| 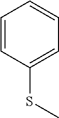 | 296.846 | | N/A |
| 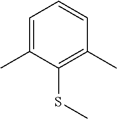 | 314.766 | | N/A |
| 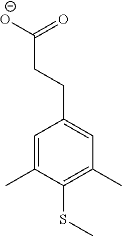 | 286.573 | 269.634 | 16.93.9 |
| 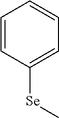 | 276.524 | | N/A |
| 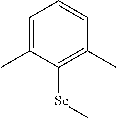 | 255.156 | | N/A |
| 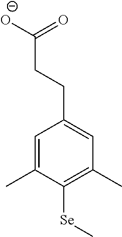 | 263.073 | 263.337 | −0.264 |
| 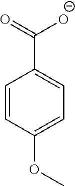 | 267.859 | 325.192 | 57.333 |
| 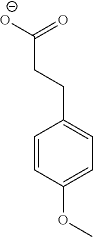 | 267.907 | 312.360 | 44.453 |
| 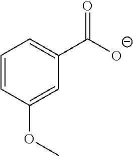 | 278.580 | 320.536 | 41.956 |
| 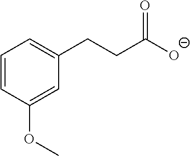 | 290.565 | 310.588 | 20.023 |
| 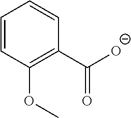 | 284.762 | 324.514 | 39.752 |
| 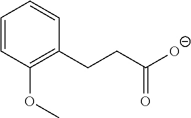 | 288.563 | 307.953 | 19.390 |
| 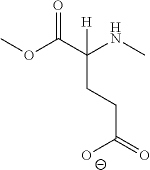 | 315.025 | 350.116 | 35.091 |
| 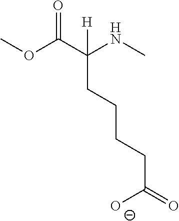 | 338.566 | 346.966 | 8.401 |
| 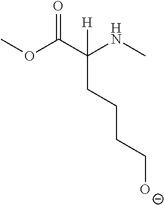 | 337.805 | 349.878 | 12.073 |
| 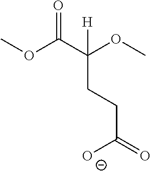 | 372.478 | 380.095 | 7.617 |
| 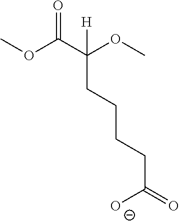 | 365.442 | 369.648 | 4.206 |
| 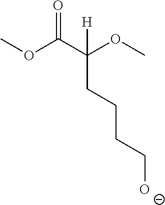 | 364.947 | 370.503 | 5.557 |
| 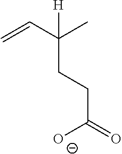 | 365.930 | 370.481 | 4.551 |
| 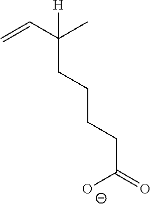 | 368.888 | 370.944 | 2.056 |
| 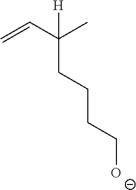 | 368.009 | 371.105 | 3.096 |
| 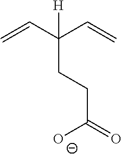 | 332.567 | 342.447 | 9.880 |
| 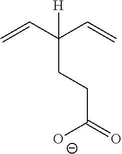 | 352.555 | 358.374 | 5.819 |
| 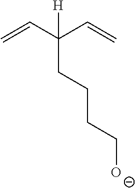 | 338.471 | 345.859 | 7.388 |
| 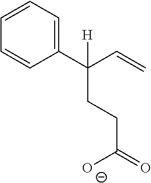 | 344.738 | 355.802 | 11.064 |
| 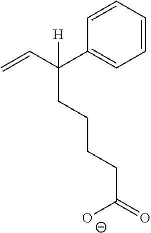 [SMILES: C=CC(CCCCC(=O)[O-])c1ccccc1] | | 358.017 | |
| 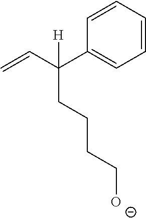 | 347.210 | 357.738 | 10.528 |
| 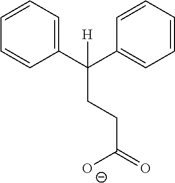 [SMILES: O=C([O-])CCC(c1ccccc1)c1ccccc1] | 369.964 | 377.964 | 8.000 |
| 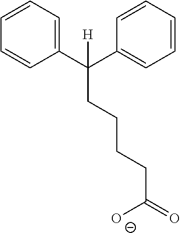 [SMILES: O=C([O-])CCCCC(c1ccccc1)c1ccccc1] | 370.761 | 374.937 | 4.176 |
| 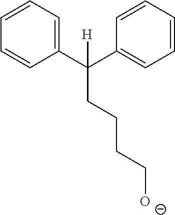 [SMILES: [O-]CCCCC(c1ccccc1)c1ccccc1] | 368.008 | 375.194 | 7.185 |
| 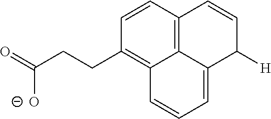 [SMILES: O=C([O-])CCc1ccc2c3c(cccc13)CC=C2] | 282.680 | 289.039 | 6.359 |
| 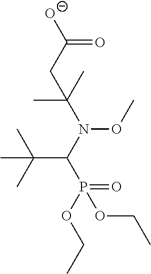 [SMILES: CCOP(=O)(OCC)C(N(OC)C(C)(C)CC(=O)[O-])C(C)(C)C] | 208.669 | 226.073 | 17.404 |
| 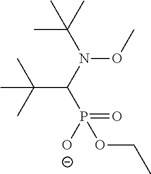 [SMILES: CCOP(=O)([O-])C(N(OC)C(C)(C)C)C(C)(C)C] | 193.295 | 225.119 | 31.824 |
| 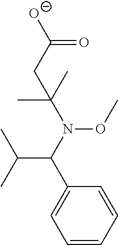 [SMILES: CON(C(c1ccccc1)C(C)C)C(C)(C)CC(=O)[O-]] | 200.792 | 217.383 | 16.591 |
| 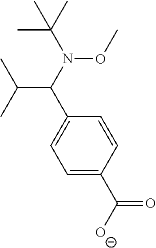 [SMILES: CON(C(c1ccc(C(=O)[O-])cc1)C(C)C)C(C)(C)C] | 209.750 | 218.659 | 8.909 |
| 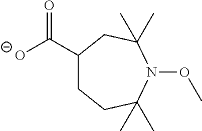 [SMILES: CON1C(C)(C)CCC(C(=O)[O-])CC1(C)C] | 210.274 | 226.452 | 16.177 |
| 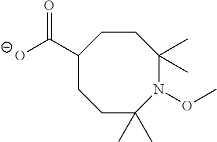 [SMILES: CON1C(C)(C)CCC(C(=O)[O-])CCC1(C)C] | 210.274 | 226.860 | 16.586 |
| 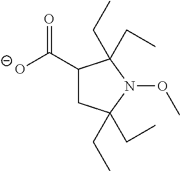 [SMILES: CCC1(CC)CC(C(=O)[O-])C(CC)(CC)N1OC] | 183.109 | 203.731 | 20.622 |
| 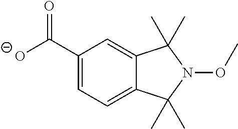 [SMILES: CON1C(C)(C)c2ccc(C(=O)[O-])cc2C1(C)C] | 204.442 | 220.190 | 15.748 |
| 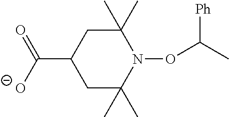 [SMILES: CC(ON1C(C)(C)CC(C(=O)[O-])CC1(C)C)c1ccccc1] | 163.821 | 182.904 | 19.1 |
| 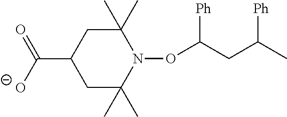 [SMILES: CC(CC(ON1C(C)(C)CC(C(=O)[O-])CC1(C)C)c1ccccc1)c1ccccc1] | 174.070 | 194.220 | 20.2 |
| 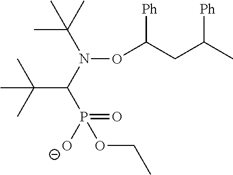 [SMILES: CCOP(=O)([O-])C(N(OC(CC(C)c1ccccc1)c1ccccc1)C(C)(C)C)C(C)(C)C] | 174.378 | 176.476 | 2.098 |
| 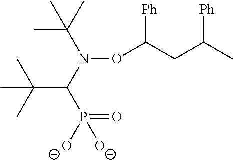 [SMILES: CC(CC(ON(C(C(C)(C)C)P(=O)([O-])[O-])C(C)(C)C)c1ccccc1)c1ccccc1] | 218.553 | 176.476 | −42.077 |

The calculations of Table 1 show BDE switching. The larger the switch value in Table 1, the more stable the radical of RAD is expected to be, and the lower the temperature at which the radical of RAD is expected to be released from a group protected by the radical of RAD, and the greater is expected the control over the orbital switching of the structure of Formula (I).

TABLE 2
Calculated kinetic and thermodynamic parameters of reactions X—H + •CH₃ →
X• + CH₄ in the gas phase at 25° C.; reactions involving switching are in bold.
^{a}Calculated using G3(MP2)-RAD(+) method in conjunction with UM06-2X/6-31 + G(d)
optimized geometries and scaled frequencies; ^{b}Rate constant, corrected for tunnelling.

| | | ΔS | ΔEₑ | Δ(Eₑ + ZPVE) | ΔH | ΔG | |
|---|---|---|---|---|---|---|---|
| | X• | J mol⁻¹ K⁻¹ | kJ mol⁻¹ | kJ mol⁻¹ | kJ mol⁻¹ | kJ mol⁻¹ | lgk |
| activation | TEMPO• | −123.523 | 22.619 | 17.879 | 14.978 | 51.806 | 6.2 |
| | COOH-TEMPO• | −123.24 | 22.688 | 18.02 | 15.086 | 51.83 | 6.2 |
| | COO⁻-TEMPO• | −126.159 | 18.117 | 13.963 | 10.978 | 48.592 | 6.5 |
| reaction | TEMPO• | −3.195 | −148.245 | −137.891 | −139.106 | −138.154 | |
| | COOH-TEMPO• | −3.411 | −146.258 | −135.938 | −137.202 | −136.185 | |
| | COO⁻-TEMPO• | −1.425 | −165.295 | −154.538 | −155.878 | −155.454 | |
| activation | PROXYL• | −120.501 | 21.611 | 16.684 | 13.885 | 49.812 | 6.5 |
| | COOH-PROXYL• | −119.652 | 21.874 | 16.968 | 14.19 | 49.865 | 6.5 |
| | COO⁻-PROXYL• | −123.966 | 16.519 | 12.386 | 9.405 | 46.366 | 6.8 |
| reaction | PROXYL• | 1.735 | −161.442 | −152.512 | −152.975 | −153.492 | |
| | COOH-PROXYL• | 8.299 | −158.378 | −149.749 | −150.128 | −152.602 | |
| | COO⁻-PROXYL• | 3.44 | −179.109 | −169.307 | −170.093 | −171.118 | |

The calculations in Table 2 consider a simple reaction of H-abstraction from carboxy-TEMPO-H and carboxy-PROXYL-H by .CH3 radical. Nitroxide radical is a product of this reaction, therefore when carboxylic group is deprotonated and the radical is stabilized by orbital conversion, free energy of the reaction is lower (reaction is more favoured) compared to when there is no switching in the product radical (the carboxylic group is protonated, or TEMPO and PROXYL are non-substituted). This effect on reaction thermodynamics is ca. 20 kJ mol⁻¹. Calculated free energy barriers of these reactions are lower when the carboxylic group is deprotonated, but this effect on kinetics is only ca. 5 kJ mol⁻¹ (or 20-25% of thermodynamics), which suggests the early character of transition states.

The inventions has shown that orbital conversion results in an increased radical stability, e.g. if such converted radical is a product of some chemical reaction, the Gibbs free energy (ΔG_{r×n}) of this reaction is lower than the Gibbs free energy of an identical reaction but involving formation of the radical species in their regular orbital configuration. Reactions involving such radical species obviously proceed through transition states (TS) that also possess some degree of radical character. If TS of a reaction is late, i.e. chemically close to the product radical (thus has a pronounced radical character) and that product radical is switched, than the TS is also expected to be switched, therefore Gibbs free energy of activation (AG^{†}) of this reaction is expected to be lower, i.e. reaction will proceed faster compared to when the product radical is not switched. And in contrast, if the TS is early and thus has a small contribution of radical character, only a fraction of switching will translate to the ΔG^{†} of this reaction. In other words, the switching in the stability of a radical that participates in a chemical reaction is expected to also affect kinetics of this reaction, but the direction and magnitude of this effect will depend on whether this radical is a reactant or a product and whether transition state of this reaction has an early or a late character. The kinetic and thermodynamics of a reaction involving the SOMO≠HOMO (switched) and SOMO=HOMO (non-switched) is illustrated in FIG. 14. FIG. 14 shows that the ΔG^{†} and the ΔG_{r×n} are both lower for the switched species than for the non-switched species. Where AG^{†} is smaller the reaction will be quicker. Where ΔG_{r×n} is lower (i.e. more exothermic) the reaction will favour products over reagents where these two species are in equilibrium.

TABLE 3
Calculated methyl BDEs in various salts of carboxy-nitroxides (in electronic
energy terms in the gas phase using UM06-2X/6-31 + G(d) method, kJ mole⁻¹).

| M⁻ in | | M²⁺ in | |
|---|---|---|---|
| 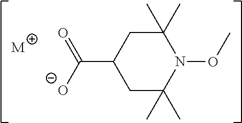 | BDE | 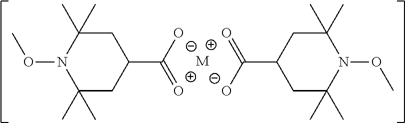 | BDE |
| NH₄⁻ | 231.3 | Mg²⁻ | |
| Li⁻ | 229.9 | Ca²⁻ | |
| Na⁻ | 228.2 | | |
| K⁻ | 226.9 | | |

The calculation in Table 3 shows that ionic bonding of a cation (e.g. lithium, sodium and potassium) stabilizes the negative charge of NEG (in a structure corresponding to Formula (I)), to the extent that the SOMO of RAD is higher in energy than the DOMOs of NEG when the anion of NEG is bonded to that cation. That is, this shows that the orbital conversion in a structure corresponding to Formula (I) can be switched off by the addition of cations to the anion of NEG.

TABLE 4
Calculated BDE-switches in the gas phase and in solvents of different polarity.
^{a}All values are at 25° C. in kJ mole⁻¹; ^{b}The breaking bond is denoted with a curly arrow;
^{c}Calculated using M06-2X/6-31 + G(d) (for carboxy-TEMPO-R and phenols) and ONIOM
approximation to the G3(MP2,CC)(+) energies at the UMP2/6-311 + G(3df,2p) level (for
TTF—CH═N-TEMPO-CH₃ using TEMPO-CH₃ as a core and a reference non-switched
system); ^{c}Calculated using UAKS-CPCM/B3LYP/6-31G(d) method in conjunction with
the recommended scaling factors (1.2 for water and 1.3 for toluene); ^{e}Calculated using
ONIOM-approximation to G3(MP2)-RAD energies at the R(O)MP2/6-311 + G(3df,2p)
level of theory; ^{f}Calculated using UAHF-CPCM/UHF/6-31 + G(d) method in conjunction
with the recommended scaling factor (1.4); ^{g}Calculated using UAKS-CPCM/B3LYP/6-
31+ G(d) method in conjunction with the recommended scaling factors (1.2 for water and
1.3 for toluene).

| Species^{b} | | | |
|---|---|---|---|
| | ΔG_{gas}^{c} | ΔG_{wate}r^{d} | ΔGₜₒₗᵤₑₙₑ^{d} |
| 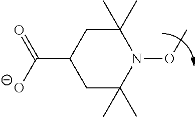 | 19.3 | 6.7 | 13.9 |
| 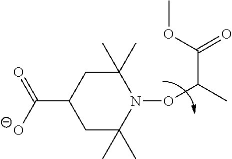 [SMILES: COC(=O)C(C)ON1C(C)(C)CC(C(=O)[O-])CC1(C)C] | 16.2 | 9.7 | 15.4 |
| 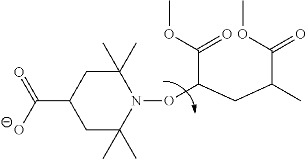 [SMILES: COC(=O)C(C)CC(ON1C(C)(C)CC(C(=O)[O-])CC1(C)C)C(=O)OC] | 15.0 | 4.8 | 16.4 |
| 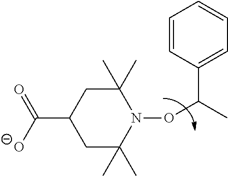 [SMILES: CC(ON1C(C)(C)CC(C(=O)[O-])CC1(C)C)c1ccccc1] | 19.3 | 10.2 | 15.7 |
| 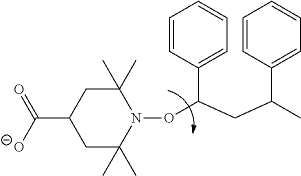 [SMILES: CC(CC(ON1C(C)(C)CC(C(=O)[O-])CC1(C)C)c1ccccc1)c1ccccc1] | 19.7 | 15.7 | 14.1 |
| 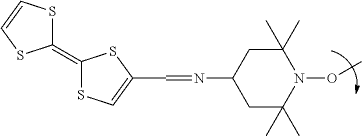 [SMILES: CON1C(C)(C)CC(N=CC2=CSC(=C3SC=CS3)S2)CC1(C)C] | 48.0 | 47.4 | 47.7 |
| | ΔG_{gas}^{g} | ΔG_{water}^{d} | ΔG_{water}^{f} |
| 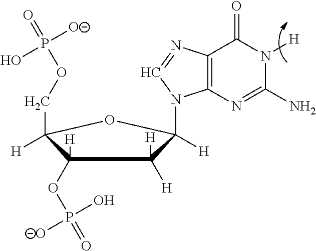 | 38.4 | −5.0 | −0.4 |
| 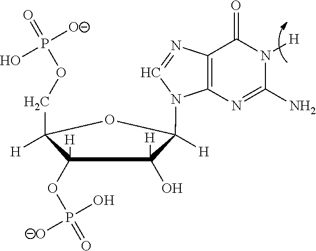 | 36.3 | −4.3 | −1.4 |
| 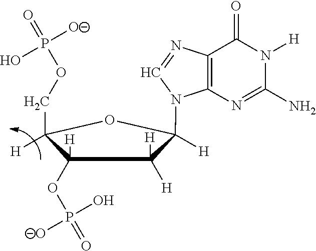 | −10.6 | −12.3 | −14.0 |
| 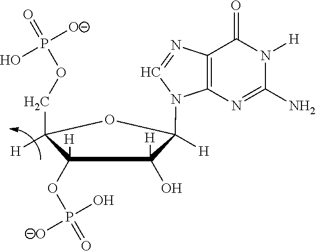 | −4.8 | −5.0 | −5.8 |
| | ΔG_{gas}^{c} | ΔG_{water}^{g} | ΔGₜₒₗᵤₑₙₑ^{g} |
| 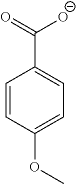 | 54.6 | 8.4 | 31.7 |
| 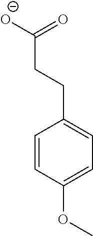 | 39.5 | 1.0 | 18.3 |
| 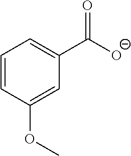 | 40.8 | 1.6 | 21.4 |
| 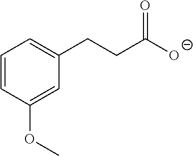 | 18.1 | −17.9 | −3.8 |
| 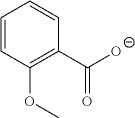 | 42.2 | 0.6 | 19.9 |
| 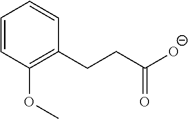 | 16.5 | −2.9 | 3.7 |

The calculations in Table 4 indicate that in polar solvents such as water orbital switching is quenched, whereas in less polar solvents like toluene it is preserved. The polarity of the solvent can therefore be used to control orbital switching of the invention. For example the polarity of a solvent can be changed by changing the constituents of the solvent, e.g. adding or removing different solvents or adding or removing polar components e.g. ammonium salts or ionic liquids.

TABLE 5
Calculated kinetic and thermodynamic parameters of reactions
of H-abstraction by •OH and •OH addition to the
base moiety in deoxyriboguanidine diphosphate
in the gas phase at 25° C., reactions involving switching are
in bold. ^{a}Calculated using ONIOM approximation to G3(MP2)-
RAD electronic energies using R(O)MP2/6-311 + G(3df,2p)
method in conjunction with UB3LYP/6-31G(d) optimized
geometries and scaled frequencies; ^{b}Fully protonated; ^{c}Doubly
deprotonated, as shown in the first column; ^{d}Position of-OH attack in

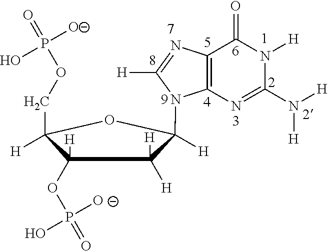

| | (H)^{da} | | (2-)^{ca} | |
|---|---|---|---|---|
| ∘ | ΔG^{•}∘ | ΔGᵣₓₙₑ | ΔG^{•}∘ | ΔGᵣₓₙ∘ |

X—H—•OH→X•—H₂Oc

| | | | | |
|---|---|---|---|---|
| 1c | wip^{d∘} | −100.814∘ | wip^{d∘} | −139.245∘ |
| 2′c | 52.390∘ | −118.687∘ | 13.263∘ | −152.908∘ |
| 8c | wip^{d∘} | −8.280∘ | wip^{d∘} | 23.269∘ |

X—•OH→X(OH)•∘

| | | | | |
|---|---|---|---|---|
| 2c | 59.527∘ | −68.141∘ | 29.055∘ | −90.505∘ |
| 4c | 35.901∘ | −39.439∘ | 33.265∘ | −40.475∘ |
| 5c | 23.083∘ | −20.746∘ | 15.627∘ | −12.635∘ |
| 8c | 30.985∘ | −64.106∘ | 64.336∘ | −22.908∘ |

TABLE 6
Comparison of the theoretical methods. Calculated electronic and Gibbs free
energies of X—CH₃ bond dissociation at 25° C., kJ mol⁻¹.

| | G3(MP2)-RAD(+) | | G3(MP2,CC)(+) | | G4(MP2)-6X | |
|---|---|---|---|---|---|---|
| X• | ΔE⁰ | ΔG²⁹⁸ | ΔE⁰ | ΔG²⁹⁸ | ΔE⁰ | ΔG²⁹⁸ |
| COO⁻—(CH₂)₄OO• | 285.222 | 213.63 | 287.751 | 216.16 | 280.124 | 208.987 |
| COOH—(CH₂)₄OO• | 298.131 | 230.165 | 300.661 | 232.694 | 293.164 | 224.822 |
| BDE-switch | 12.91 | 16.535 | 12.91 | 16.534 | 13.04 | 15.835 |
| COO⁻-TEMPO• | 211.496 | 137.578 | 215.28 | 141.362 | | |
| COOH-TEMPO• | 230.47 | 156.86 | 233.914 | 160.304 | | |
| BDE-switch | 18.973 | 19.281 | 18.634 | 18.942 | | |

### Homologues Series

### Methodology.

To investigate the nature of the pH-switching in the trial carboxy peroxyl and aminoxyl radicals, these were paired with the structurally similar carboxy alkoxyl radicals which do not display an orbital conversion upon deprotonation. The ‘test set’ were also expanded to the series of the homologues in which there was varied the length of the (CH₂)ₙ chains separating the radical and carboxylic moieties (see FIG. 3 and Example 7a). These chains were kept in their extended conformations in order to study the 1/r dependence of the calculated bond energetics, where r is the (through-space) distance between the radical and carboxylic moieties.

Accurate geometries of all species were obtained using M06-2X/6-31+G(d,p) method. For the converted acyclic peroxyl series and the reference alkoxyl series, there was also performed geometry optimizations using BMK/6-31+G(2df,p) method (as part of the G4(MP2)-6X procedure). In the case of alkoxyl radicals and corresponding ethers the conformations of the terminal units were altered in order to achieve the local minima (e.g., without imaginary frequencies, Example 7b) in several cases which resulted in the slight deviations from the linearity of their bond energetics (see below). Additionally, there was performed a full conformational search for the aminoxyl homologue with n=4 in the gas-phase using UM06-2X/6-31+G(d) method in order to evaluate the effect of the conformation on the bond energetics in both protonation states of the COOH group (see Example 7c). Finally, there was constructed a supermolecule containing the TEMPO radical and an acetic acid, non-bonded to it, such that an aminoxyl radical moiety and a negative charge are spatially located exactly as in the n=4 extended chain homologue of the carboxy-aminoxyl series (Example 7d). This allowed further investigation of the nature of the orbital conversion effect (through-bond or through-space) and the contribution of the σ-assistance to the BDE-switch.

Next, calculated was the absolute BDEs of the homologues series in the electronic energy terms using a variety of quantum-chemical methods (Table 7 and Table 8). As noted earlier, not all methods were applied to the cyclic homologues series due to their large size, and a two-layer ONIOM approximation was used to obtain high-level energies. Results, obtained using unrestricted and restricted open-shell wave functions differ by no more than 4 kJ mol⁻¹; the difference between the G3 and G4 results is only marginally larger (ca. 7 kJ mol⁻¹); generally, the M06-2X method is in an excellent agreement with the G3 results; MP2 values are generally overestimated compared to G3 and G4, yet this deviation appears to be systematic. On the other hand and not surprisingly, B3LYP results are off by 10s of kJ mol⁻¹ compared to the composite methods. Furthermore, BDEs of all the protonated species are generally independent on the separation (n) between the radical and carboxylic moieties; the same is largely true for the deprotonated reference alkoxyl radicals, whereas BDEs of the SOMO-HOMO converted species in the deprotonated state increase slowly with separation. Interestingly, bond energies in the deprotonated form of the lowest-energy conformer of aminoxyl n=4 homologue follows the 1/r linear dependence, exhibited by all the extended-chain conformers (see

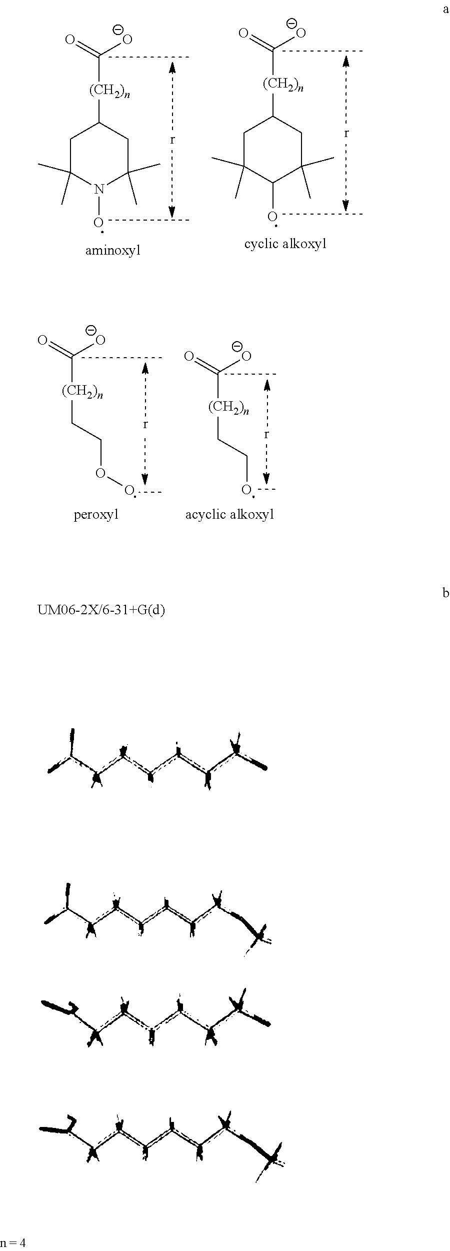

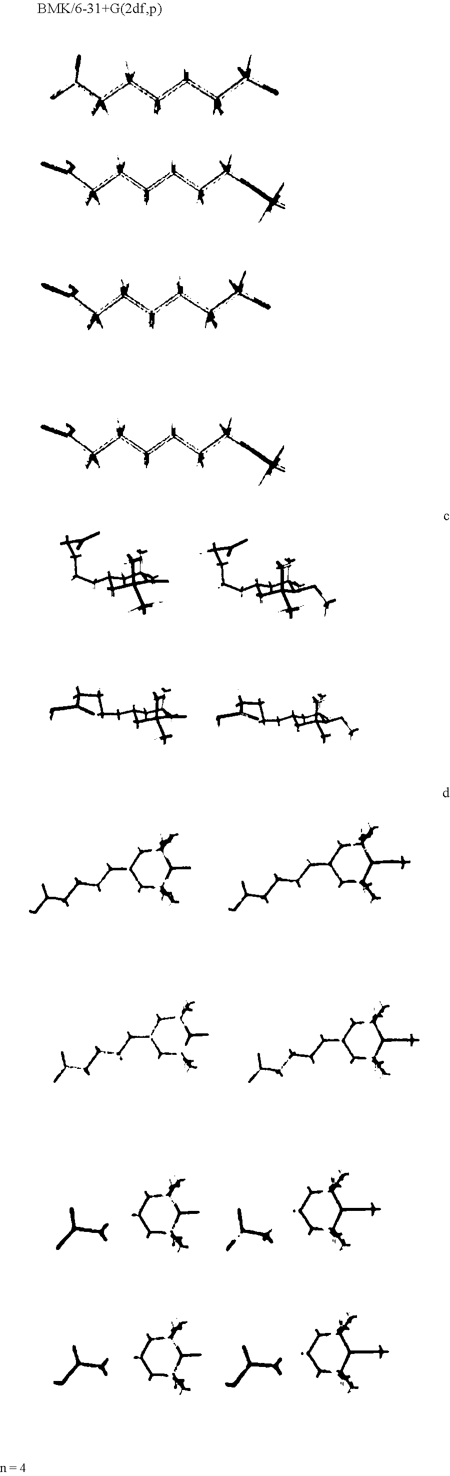

Example 8). The BDE in its protonated form is slightly lower than in the other homologues due to a weak hydrogen bonding interaction; as this interaction is not possible in the corresponding deprotonated species the pH-switch on its BDE is slightly diminished (see Example 7c) Nonetheless, the methyl BDE of the supermolecule with the deprotonated acetic acid is less than 1.5 kJ mol⁻¹ higher than that of an extended chain n=4 homologue

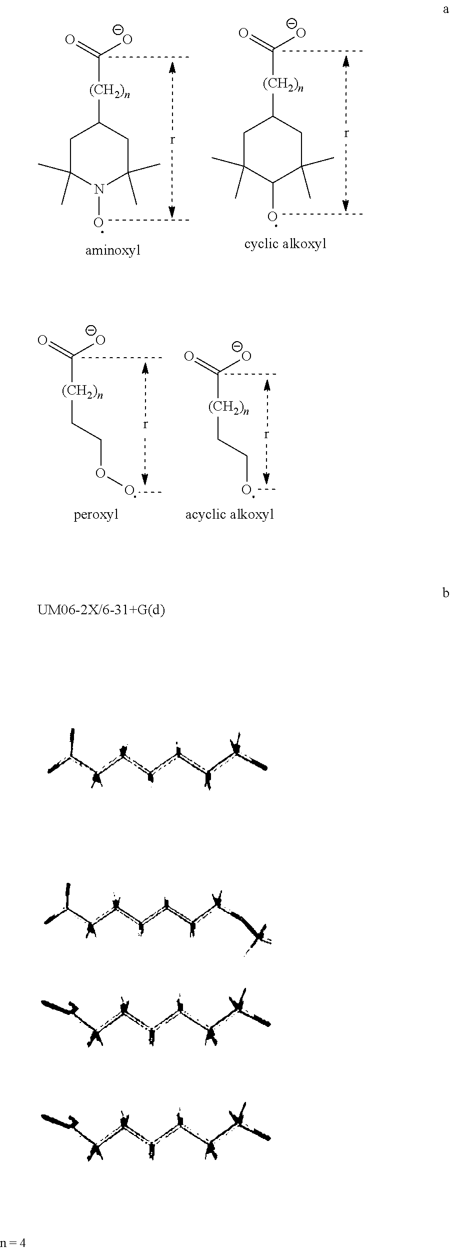

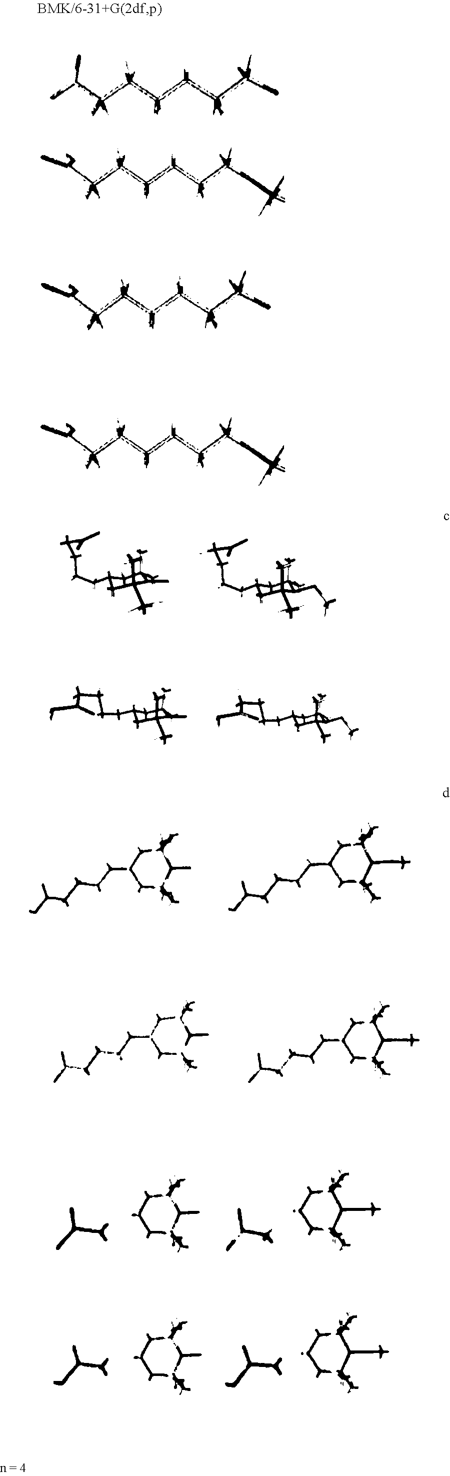

(Example 8), thus confirming the primarily through-space nature of the orbital conversion switching effect and only a very minor contribution from the through-bond σ-assistance.

### Example 7

### Structures of the Homologues. a

Homologue series of converted carboxy aminoxyl and -peroxyl radicals and their reference cyclic and acyclic carboxy alkoxyl radicals. b, Optimized M06-2X/6-31+G(d) and BMK/6-31+G(2df,p) geometries of the n=4 homologues in the acyclic alkoxyl series (shown are radical species and the corresponding methyl ethers in the deprotonated and protonated forms of the carboxylic group). Optimized M06-2X/6-31+G(d) geometries of the lowest energy c, and extended chain, d (top), conformers of the aminoxyl homologue with n=4, as well as the supermolecule of TEMPO and acetic acid, d (bottom); shown are the radical species and the corresponding methyl ethers in the deprotonated and protonated forms of the carboxylic group.

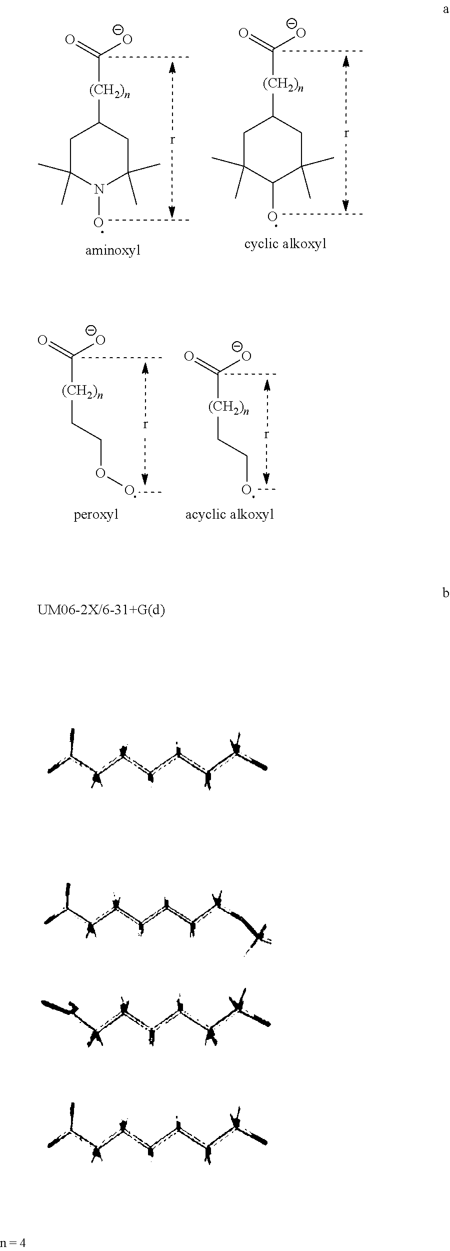

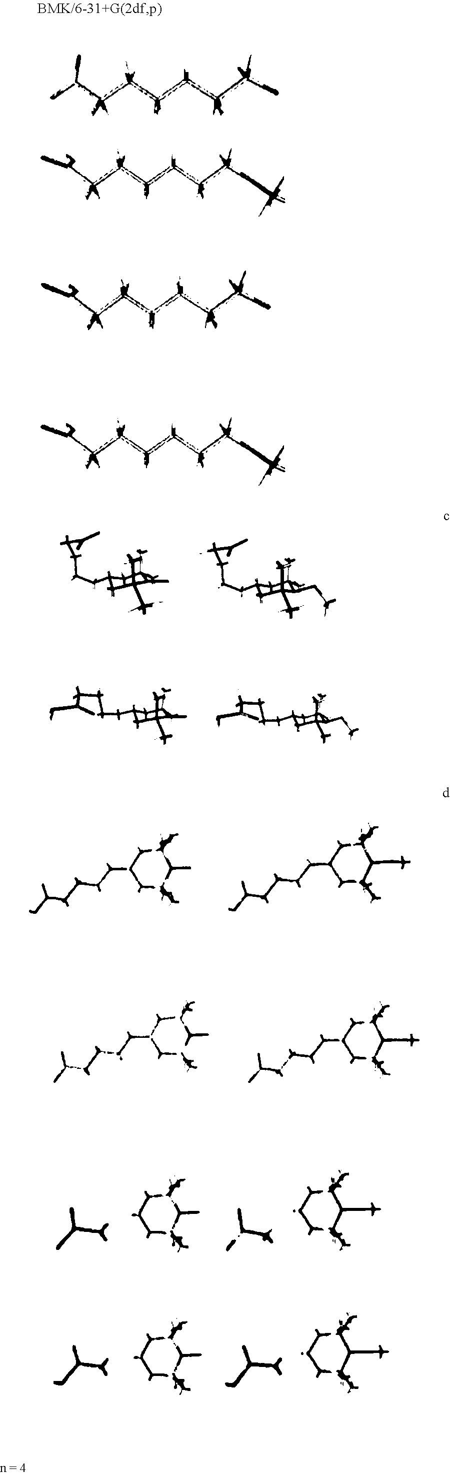

### Example 8

### BDEs of the Aminoxyl Homologues

Calculated M06-2X/6-31+G(d) bond dissociation electronic energies of the aminoxyl homologues series: extended chain conformers (∘), lowest gas-phase energy conformer of n=4 (□) and supermolecule consisting of TEMPO and acetic acid (⋄).

TABLE 7
BDEs of the acyclic homologues series. Calculated electronic energies of
X—CH₃ bond homolyses for the acyclic trial peroxyl and reference alkoxyl
radicals in both protonation states of the carboxylic group, kJ mol⁻¹.

| | | | | | | | |
|---|---|---|---|---|---|---|---|
| n^{a} | B3LYP^{b} | M06-2X^{b} | UMP2^{c} | CCSD(T)^{b} | G3-RAD^{d} | G3-CC^{e} | G4^{f} |

Deprotonated peroxyl series

| | | | | | | | |
|---|---|---|---|---|---|---|---|
| 0 | 247.898 | 283.991 | 312.208 | 265.287 | 279.515 | 282.033 | 276.004 |
| 1 | 251.739 | 287.401 | 314.353 | 268.697 | 281.771 | 284.302 | 277.302 |
| 2 | 255.403 | 291.11 | 318.033 | 272.131 | 285.222 | 287.751 | 280.124 |
| 3 | 258.002 | 293.634 | 320.475 | 274.744 | 287.526 | 290.056 | 281.791 |

Protonated peroxyl series

| | | | | | | | |
|---|---|---|---|---|---|---|---|
| 0 | 272.225 | 307.318 | 334.08 | 288.16 | 300.553 | 303.078 | 296.95 |
| 1 | 270.746 | 305.681 | 332.427 | 286.959 | 298.98 | 301.509 | 294.713 |
| 2 | 269.598 | 304.745 | 331.525 | 285.806 | 298.131 | 300.661 | 293.164 |
| 3 | 269.176 | 304.322 | 331.067 | 285.506 | 297.71 | 300.24 | 292.062 |

Deprotonated alkoxyl series

| | | | | | | | |
|---|---|---|---|---|---|---|---|
| 1 | 308.607 | 364.682 | 395.198 | 339.339 | 357.878 | 360.317 | 358.267 |
| 2 | 329.646 | 378.476 | 405.181 | 350.064 | 371.511 | 373.9 | 371.465 |
| 3 | 320.177 | 372.584 | 398.61 | 345.219 | 365.336 | 367.731 | 361.84 |
| 4 | 318.973 | 372.846 | 398.804 | 345.427 | 365.678 | 367.023 | 366.708 |

Protonated alkoxyl series

| | | | | | | | |
|---|---|---|---|---|---|---|---|
| 1 | 335.204 | 374.596 | 400.529 | 346.505 | 367.816 | 370.194 | 367.509 |
| 2 | 343.944 | 381.523 | 407.915 | 353.566 | 374.938 | 377.321 | 374.835 |
| 3 | 335.485 | 375.208 | 401.033 | 347.043 | 368.207 | 370.59 | 366.55 |
| 4 | 336.649 | 377.174 | 402.938 | 349.716 | 370.193 | 371.538 | 367.815 |

^{a}Refers to structures in Example 7a;
^{b}6-31 + G(d) basis set;
^{c}GTMP2Large basis set;
^{d}G3-RAD stands for G3(MP2)-RAD(+) method;
^{e}G3-CC stands for G3(MP2,CC)(+);
^{f}G4 stands for G4(MP2)-6X method.

TABLE 8
BDEs of the cyclic homologues series. Calculated electronic energies of
X—CH₃ bond homolyses for the cyclic trial aminoxyl and reference alkoxyl
radicals in both protonation states of the carboxylic group, kJ mol⁻¹.

| | | M06- | | | G3- | | ONIOM^{f} | |
|---|---|---|---|---|---|---|---|---|
| n^{a} | B3LYP^{b} | 2X^{b} | UMP2^{c} | R(O)MP2^{c} | RAD^{d} | G3-CC^{e} | A | B |

Deprotonated aminoxyl series

| | | | | | | | | |
|---|---|---|---|---|---|---|---|---|
| 0 | 164.038 | 214.162 | 225.423 | 206.797 | 211.496 | 215.28 | 211.629 | |
| 1 | 166.626 | 216.435 | 228.164 | 210.06 | 213.895 | 217.66 | 214.37 | 218.021 |
| 2 | 170.05 | 220.36 | 232.433 | 215.174 | | | 218.639 | 222.291 |
| 3 | 171.939 | 222.25 | 234.846 | 218.061 | | | 221.052 | 224.703 |
| 4 | 173.591 | 223.997 | 236.756 | 220.413 | | | 222.963 | 226.614 |
| 5 | 174.781 | 225.183 | 238.346 | 222.306 | | | 224.552 | 228.203 |
| 6 | 175.719 | 226.219 | 239.533 | 223.737 | | | 225.739 | 229.39 |
| 7 | | 226.912 | | | | | | |
| 8 | | 227.473 | | | | | | |
| 9 | | 228.013 | | | | | | |
| 10 | | 228.429 | | | | | | |
| 4c^{g} | 165.817 | 214.775 | 227.938 | 209.675 | | | 214.144 | 217.795 |
| 4s^{h} | | 225.329 | | | | | | |

Protonated aminoxyl series

| | | | | | | | | |
|---|---|---|---|---|---|---|---|---|
| 0 | 182.885 | 233.184 | 248.06 | 234.166 | 230.47 | 233.914 | 234.266 | |
| 1 | 181.457 | 231.65 | 246.24 | 231.916 | 229.054 | 232.529 | 232.446 | 232.094 |
| 2 | 181.637 | 232.098 | 246.524 | 232.196 | | | 232.731 | 232.379 |
| 3 | 181.229 | 231.676 | 245.991 | 231.54 | | | 232.197 | 231.845 |
| 4 | 181.066 | 231.554 | 245.863 | 231.377 | | | 232.069 | 231.718 |
| 5 | 180.928 | 231.393 | 245.819 | 231.283 | | | 232.025 | 231.674 |
| 4c^{g} | 180.034 | 229.693 | 244.158 | 229.259 | | | 230.364 | 230.012 |
| 4s^{h} | | 231.564 | | | | | | |

Deprotonated alkoxyl series

| | | | | | |
|---|---|---|---|---|---|
| 0 | 311.672 | 362.441 | 396.124 | 360.7 | |
| 1 | 310.906 | 362.904 | 396.559 | | 361.136 |
| 2 | 308.07 | 365.317 | 398.261 | | 362.837 |
| 3 | 306.426 | 366.205 | 399.182 | | 363.758 |
| 4 | 303.361 | 367.16 | 400.099 | | 364.676 |
| 5 | 301.831 | 367.785 | 400.579 | | 365.155 |

Deprotonated alkoxyl series

| | | | | | |
|---|---|---|---|---|---|
| 0 | 323.42 | 372.361 | 404.722 | 369.829 | |
| 1 | 323.027 | 372.05 | 404.433 | | 369.54 |
| 2 | 322.951 | 371.971 | 404.454 | | 369.561 |
| 3 | 322.618 | 371.749 | 404.158 | | 369.265 |
| 4 | 322.359 | 371.424 | 403.942 | | 369.049 |
| 5 | 322.21 | 371.339 | 403.652 | | 368.759 |

^{a}Refers to structures in Example 7a;
^{b}6-31 + G(d) basis set;
^{c}6-311 + G(3df,2p) basis set;
^{d}G3-RAD stands for G3(MP2)-RAD(+) method;
^{e}G3-CC stands for G3(MP2,CC)(+);
^{f}ONIOM approximation using non-substituted TEMPO (A) or the first homologue (n = 0, B) treated at G3(MP2, CC(+) level of theory as a core for higher homologues treated at UMP2/6-311 + G(3df,2p) level;
^{g}Lowest-energy conformer, see Example 7c:
^{h}Supermolecule, see Example 7d, energies are corrected for the Basis Set Superposition Error (BSSE) using the counterpoise correction.

In order to illustrate the interaction between the negative charge and the radical, there was also calculated the BDE-switches (see Table 9). In addition to the methods, listed in Table 7 and Table 8, there was used standard DFT-D3 protocol with BJ damping to assess the dispersion contribution to the B3LYP BDE-switches and found it to be marginal. All methods, except for the B3LYP, are in a good agreement and indicate a significantly larger switch in the trial species compared to their references at the same separation. The ONIOM approximation using the first homologue as a core appears to underestimate the BDE-switch of the aminoxyl series as compared to the UM06-2X and UMP2 results. The difference between the UMP2 and G3(MP2,CC)(+) values also depends linearly on 1/r (Table 8), while ONIOM corrects for the constant value, the largest within the series for the first homologue used as an ONIOM core, which causes the underestimation. Therefore, in FIG. 3 the ONIOM values based instead on the non-substituted TEMPO as a core for both the protonated and deprotonated series (effectively equal to UMP2 for the BDE-switches).

TABLE 9
BDE-switches. Calculated BDE-switches in electronic energy terms for trial and
reference series, kJ mol⁻¹.

| | | | | | | | | |
|---|---|---|---|---|---|---|---|---|
| | | | | M06- | | | | |
| n^{a} | l/r, Å | Dispersion | B3LYP^{b} | 2X^{b} | Exchange^{c} | HF/CBS^{d} | G3-CC^{e} | G4^{f} |

Acyclic carboxy-peroxyl series

| | | | | | | | | |
|---|---|---|---|---|---|---|---|---|
| 0 | 0.225 | 0.211 | 24.327 | 23.326 | 7.159 | 15.464 | 21.045 | 20.946 |
| 1 | 0.163 | −0.463 | 19.008 | 18.28 | 0.541 | 13.918 | 17.207 | 17.411 |
| 2 | 0.135 | −0.375 | 14.195 | 13.635 | 2.24 | 10.587 | 12.91 | 13.04 |
| 3 | 0.115 | −0.296 | 11.174 | 10.688 | 1.015 | 8.341 | 10.183 | 10.271 |

Acyclic carboxy-alkoxyl series

| 1 | 0.202 | −0.433 | 26.596 | 9.914 | 9.232 | 3.696 | 9.877 | 9.242 |
|---|---|---|---|---|---|---|---|---|
| 2 | 0.178 | −0.13 | 14.298 | 3.047 | 8.69 | 3.026 | 3.421 | 3.37 |
| 3 | 0.131 | −0.538 | 15.308 | 2.624 | 6.008 | 3.706 | 2.86 | 4.71 |
| 4 | 0.114 | −0.067 | 17.676 | 4.328 | 7.951 | 4.018 | 4.514 | 1.108 |
| | | | | M06- | | | UMP2/ | |
| | | Dispersion | B3LYP^{b} | 2X^{b} | Exchange^{c} | HF/TZ^{g} | TZ^{g} | ONIOM^{h} |

Cyclic carboxy-aminoxyl series

| | | | | | | | | |
|---|---|---|---|---|---|---|---|---|
| 0 | 0.176 | −0.06 | 18.847 | 19.022 | 6.648 | 18.767 | 22.637 | 18.634 |
| 1 | 0.152 | 0.079 | 14.831 | 15.215 | 5.281 | 14.742 | 18.076 | 14.073 |
| 2 | 0.124 | 0.037 | 11.587 | 11.738 | 3.019 | 11.193 | 14.091 | 10.088 |
| 3 | 0.11 | 0.03 | 9.29 | 9.426 | 2.239 | 8.977 | 11.145 | 7.142 |
| 4 | 0.095 | 0.018 | 7.475 | 7.557 | 1.666 | 7.181 | 9.106 | 5.104 |
| 4cⁱ | 0.163 | 0.254 | 14.217 | 14.918 | 8.749 | 13.83 | 16.22 | 12.217 |
| 4s^{j} | 0.094 | | | 6.235 | | | | |
| 5 | 0.086 | 0.011 | 6.147 | 6.21 | 1.178 | 5.886 | 7.473 | 3.47 |

Cyclic carboxy-alkoxyl series

| | | | | | | | | |
|---|---|---|---|---|---|---|---|---|
| 0 | 0.174 | 0.005 | 11.747 | 9.92 | 7.902 | 9.105 | 8.598 | 9.128 |
| 1 | 0.15 | 0.091 | 12.121 | 9.145 | 7.624 | 8.207 | 7.874 | 8.404 |
| 2 | 0.122 | 0.037 | 14.88 | 6.655 | 4.557 | 6.06 | 6.193 | 6.724 |
| 3 | 0.109 | 0.054 | 16.192 | 5.544 | 3.98 | 4.978 | 4.976 | 5.507 |
| 4 | 0.094 | 0.017 | 18.998 | 4.263 | 2.547 | 3.88 | 3.843 | 4.373 |
| 5 | 0.085 | 0.01 | 20.38 | 3.554 | 2.134 | 3.245 | 3.073 | 3.604 |

^{a}Refers to structures in Example 7a;
^{b}6-31 + G(d) basis set;
^{c}Sum of alpha and beta exchanges, calculated at the same basis, as the overall HF component;
^{d}HF energy extrapolated to the complete basis set (HF/GTMP2Large values for the M06-2X geometries are given for the alkoxyl set);
^{e}G3-CC stands for G3(MP2,CC)(+) method;
^{f}G4 stands for G4(MP2)-6X method;
^{g}TZ stands for the 6-311 + G(3df,2p) basis set;
^{h}ONIOM approximation using the first homologue (n = 0)treated at G3(MP2,CC)(+) level of theory as a core for higher homologues treated at UMP2/6-311 + G(3df,2p) level;
ⁱLowest-energy conformer, see Example 7c;
^{j}Supermolecule, see Example 7d, energies are corrected for the BSSE using the counterpoise correction.

### Gas-Phase Acidities

### Thermocycle.

Experimental verification of the results is based upon the measurements of the relative gas-phase acidities (in enthalpic terms) for the pairs of carboxylated aminoxyls and alkoxyamines, which can be directly compared to the calculated enthalpies of bond dissociation in these alkoxyamines according to a thermocycle given in FIG. 5a. Derivation of the resulting relationship is shown in Example 9 below.

### Example 9

### Thermocycle Derivation

Derivation of the relationship between the BDE- and GPA-switches.

| | |
|---|---|
| BDE(OOC—NOR) • ΔH(OOC—NO•) + ΔH(R•)−ΔH(OOC—NOR) | GPA(HOOC—NOR) • ΔH(HOOC—NOR) − ΔHt• |
| BDE(HOOC—NOR) • ΔH(HOOC—NO•) • ΔH(R•) − ΔH(HOOC—NOR) | GPA(HOOC—NO•) • ΔH(HOOC—NO•) • ΔH(OOC—NO•) − ΔH• |
| BDE(HOOC—NOR) − BDE(OOC—NOR) | GPA(HOOC—NO•) − GPA(HOOC—NOR) |
| −ΔH(HOOC—NO•) + ΔH(R•) − ΔH(HOOC—NOR) − | −ΔH(HOOC—NO•) • ΔH(OOC—NO•) • ΔH•) |
| •ΔH(OOC—NO•) − ΔH(R•) + ΔH(OOC—NOR) • | ΔH(HOOC—NOR) • ΔH(OOC—NOR) • ΔH• |
| −ΔH(HOOC—NO•) − ΔH(HOOC—NOR) − ΔH(OOC—NO•) − | •ΔH(HOOC—NO•) • ΔH(OOC—NO•) − H(HOOC—NOR)• |
| ΔH(OOC—NOR) | ΔH(OOC—NOR) |

BDE(HOOC—NOR) − BDE(OOC—NOR) − GPA(HOOC—NO•) − GPA(HOOC—NOR)

### Experimental Gas Phase Acidity Determinations Using the Kinetic Method.

Experiments were performed for the pairs of compounds from a set given in Example 10. All reagents, including 4-carboxy-2,2′,6,6′-tetramethylpiperidine-N-oxyl radical (carboxy-TEMPO) and 3-carboxy-2,2′,5,5′-tetramethylpyrrolidine-N-oxyl radical (carboxy-PROXYL), were purchased from Sigma Aldrich (Castle Hill, Australia) and used as received unless noted otherwise. All alkoxyamines were prepared from their aminoxyl radical precursors by the standard literature procedures. Methanol for mass spectrometry was HPLC grade, and purchased from Crown Scientific (now VWR, Queensland, Australia) and used as received.

### Example 10

### Test Set

Aminoxyls and alkoxyamines, studies in the mass-spec experiments.

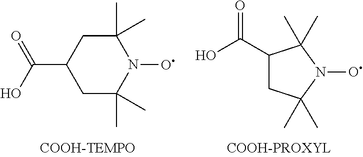

A COOH-PROXYL
B COOH-PROXYL-CH₃
C COOH-PROXYL-CH₂F
D COOH-PROXYL-CH₂Ph
E COOH-TEMPO
F COOH-TEMPO-CH₃
G COOH-TEMPO-CH₂F
H COOH-TEMPO-CH₂Ph

Collision-induced dissociation mass spectra obtained from proton-bound dimers of the form (A⁻+H⁺+B⁻)⁻, where A⁻ and B⁻ represent any pair of conjugate bases formed from aminoxyl radicals and/or related alkoxyamines, were obtained as described in the manuscript. The ratio of product ion abundances [A⁻] and [B⁻] in the resulting mass spectra are provided in Table 10. Application of the kinetic method to these data as summarized in Equation 1, allows the ion abundance ratios to be used to derive the difference in the gas phase acidities (formally, enthalpies of deprotonation given by Δ_{acid}H₂−A_{acid}H₁) of the two conjugate acids. The kinetic method relationship relies on: (i) the absence of reverse activation barriers; (ii) the absence of any isomer forms of the cluster; (iii) a negligible entropy difference for the competitive dissociation channels (i.e., Δ(Δ_{acid}G) simplifies to Δ(Δ_{acid}H)). Assumptions (i) and (ii) are generally considered as robust for dissociation of simple proton bound dimers and for the systems studied here only simple dimer dissociation was observed suggesting (iii) also holds for the experiments.

TABLE 10
Mass spec abundance ratios.

| I₁ I₂ | A | B | C | D | E | F | G | H |
|---|---|---|---|---|---|---|---|---|
| A | | 45.41 | 12.51 | 38.1 | 6.93 | 175.55 | 60.13 | 111.77 |
| B | | | 0.18 | 0.33 | 0.14 | — | — | — |
| C | | | | 3.4 | 0.61 | — | 5.96 | — |
| D | | | | | 0.16 | — | — | — |
| E | | | | | | 15.58 | 8.43 | 22.15 |
| F | | | | | | | — | 0.32 |
| G | | | | | | | | — |
| H | | | | | | | | |
| Benzoic | — | — | — | — | 0.097 | 4.381 | — | — |
| Acid | | | | | | | | |

Ion abundance ratios (I₁:I₂), obtained from the collision-induced dissociation of proton-bound dimers at a collision energy of 10 V (in the laboratory frame) in a triple quadrupole mass spectrometer using argon as the target gas. Reported ratios are the average of at least 200 cumulative scans and include replicate infusions of samples solutions. A ratio greater than unity implies Δ_{acid}H₁>Δ_{acid}H₂. Ratios of primary importance are those comparing aminoxyl radicals (A & E) to alkoxyamines. Selected additional ratios are reported between alkoxyamines pairs as a cross-reference for internal consistency.

$$ln ( \frac{{k}_{1}}{{k}_{2}} ) = ln ( \frac{[ {M}_{1}^{-} ]}{[ {M}_{2}^{-} ]} ) \approx \frac{Δ {H}_{2} - Δ {H}_{1}}{{RT}_{eff}} + \frac{Δ ( Δ S )}{R} \qquad Equation 1$$

In Equation 1, k₁ and k₂ are dissociation rate constants; Δ(ΔS) is the difference in entropy between the competing dissociation pathways; R is the universal gas constant, and T_{eff} is the effective temperature parameter. The latter is an empirical scaling factor that accounts for the non-Boltzmann energy distribution within the activated dimer population and is not a true temperature. In a related study, there was used an approach to prepared dimers from carboxy-TEMPO and a range of reference mono-substituted benzoic acids, and subjected them to collision induced dissociation over a range of collision energies from 3-30V in the laboratory frame. The logarithm of the intensity ratio is plotted against a relative enthalpy scale for each collision energy, the slope of such a plot being equal to −1/RT_{eff}. Therefore there can be derived an effective temperature for each collision energy employed, as shown in Example 11, where collision energies have been normalised by the reduced mass. Based on these experiments there can be consistently employed an effective temperature of 600 K. Using this approach with the ion abundances in Table 10 there can be derived the differences in enthalpies of deprotonation (Δ_{acid}H₂−A_{acid}H₁) for a suite of pairwise combinations of aminoxyl radicals with their closed-shell alkoxyaminederivatives. These values are referred to here as GPA-switch energies and are listed in Table 11. In the same way product ion ratios arising from dimers of benzoic acid with each of COOH-TEMPO and 4-carboxy-1-methoxy-2,2,6,6-tetramethylpiperidine (COOH-TEMPO-CH₃) (Table 10) yield GPA-switch energies of −11.6 6 kJ mol⁻¹ and 7.4 6 kJ mol⁻¹, respectively. Using the reported gas phase enthalpy of deprotonation of benzoic acid of 1423±9 kJ mol⁻¹ there can thus be assigned the gas phase acidities of Δ_{acid}H(COOH-TEMPO)=1411 kJ mol⁻¹ and Δ_{acid}H(COOH-TEMPO-CH₃)=1430 kJ mol⁻¹ with absolute uncertainties similar to those of the reference compound.

### Example 11

### Determination of the Effective Temperature

Effective temperature dependence on collision energy in the centre of mass frame for the described instrument setup, using multiple benzoic acids as reference compounds.

TABLE 11
Experimental GPA- and theoretical BDE-switches. Experimental
GPA-switches and calculated BDE-switches (gas-phase enthalpies
at 25° C., kJ mol⁻¹) for the pairs of aminoxyls and
alkoxyamines from Example 10.

| Pair | GPA-switch | BDE-switch | Absolute Δ |
|---|---|---|---|
| A:B | −19 | −19.9 | 0.9 |
| A:C | −12.6 | −9.1 | 3.5 |
| A:D | −18.2 | −17.5 | 0.7 |
| A:E | −9.7 | −8.9 | 0.8 |
| A:F | −25.8 | −27.2 | 1.4 |
| A:G | −20.4 | −19.5 | 0.9 |
| A:H | −23.5 | −22.4 | 1.1 |
| B:C | 8.4 | 10.7 | 2.3 |
| B:D | 5.5 | 2.3 | 3.2 |
| B:E | 10 | 10.9 | 0.9 |
| C:D | −6.1 | −8.4 | 2.3 |
| C:E | 2.5 | 0.2 | 2.3 |
| C:G | −8.9 | −10.4 | 1.5 |
| D:E | 9.1 | 8.6 | 0.5 |
| E:F | −13.7 | −18.2 | 4.5 |
| E:G | −10.6 | −10.5 | 0.1 |
| E:H | −15.5 | −13.5 | 2 |
| F:H | 5.7 | 4.7 | 1 |
| | | Max dev. | 4.5 |
| | | Mean dev. | 1.7 |

### Theoretical Calculations.

Correspondingly, absolute GPAs and BDEs of all the species were calculated in the gas-phase at 25° C. (Table 12) using G3(MP2)-RAD(+) method or an ONIOM approximation to it, as described above. For the GPA calculations, the thermochemistry of a proton was taken according to the electron convention under Fermi-Dirac statistics. Finally, there was calculated the BDE-switches of the various pairs of these species and compared them to the experimentally determined GPA-switches (Table 11). Obtained theoretical and experimental results are in an excellent agreement and confirm that in all cases aminoxyl radicals are more acidic than their closed-shell alkoxyamines, or from the radical stability perspective, deprotonation of the COOH group lowers the BDE in alkoxyamines.

TABLE 12
Calculated absolute GPAs and BDEs. Calculated absolute GPAs
and BDEs of the species in Example 10 (gas phase enthalpies
at 25° C., kJ mol⁻¹).

BDE

| Species | GPA | protonated | deprotonated |
|---|---|---|---|
| A | 1405.166 | | |
| B | 1425.026 | 197.913 | 178.052 |
| C | 1414.281 | 250.272 | 241.157 |
| D | 1422.702 | 182.093 | 164.557 |
| E | 1414.093 | | |
| F | 1432.338 | 209.631 | 191.386 |
| C | 1424.637 | 255.586 | 245.042 |
| H | 1427.591 | 182.767 | 169.269 |

### Derivation of Reaction Efficiencies.

Reaction efficiencies for oxygen addition to cyclohexyl radicals were estimated by comparing the measured second order reaction rate constant to the hard sphere collision rate (kₕₛ) using

$${k}_{hs} = π {{σ}_{AB}^{2} ( \frac{8 {k}_{B} T}{π μ} )}^{2} ,$$

where σ_{AB}²=(R_{A}+R_{B})² and R_{A} and R_{B} are the molecular radii of cyclohexyl radical and dioxygen taken as 1.74 Å and 2.90 Å, respectively, k_{B} is the Boltzmann constant, T is absolute temperature and μ is the reduced mass

$$( \frac{{m}_{A} * {m}_{B}}{{m}_{A} + {m}_{B}} ) .$$

TABLE 13
Methyl BDEs of TEMPO with various anionic groups. Calculated
bond dissociation electronic and free energies (in the gas phase at
25° C., kJ mol−1) for the species in FIG. 6.

| | | UM06-2X/ | | |
|---|---|---|---|---|
| | Protonation | 6-31 + G(d) | G3(MP2, CC)(+) | |
| Species | state^{a} | ΔE | | ΔG |
| Phosphate- | (—) | 217.532 | 217.009 | 143.676 |
| TEMPO-CH₃ | (H) | 232.93 | 232.291 | 158.858 |
| Sulfate- | (—) | 218.135 | 217.792 | 144.181 |
| TEMPO-CH₃ | (H) | 235.127 | 234.638 | 160.511 |
| Alkoxide- | (—) | 209.12 | 209.213 | 135.969 |
| TEMPO-CH₃ | (H) | 232.834 | 232.062 | 158.174 |
| Thiocarboxylate- | (—) | 216.107 | 213.38 | 139.888 |
| TEMPO-CH₃ | (H) | 233.688 | 230.836 | 157.721 |

TABLE 14
BDEs of the nucleic acid models. Calculated bond dissociation
electronic energies (gas phase, kJ mol⁻¹) for the
species in FIG. 6b.

| | Protonation state^{a} | UB3LYP/6-31G(d) | ONIOM^{b} |
|---|---|---|---|
| DNA | (—) | 411.705 | 433.714 |
| sugar | (H) | 403.871 | 434.57 |
| radical | | | |
| DNA | (—) | 356.729 | 387.121 |
| base | (H) | 400.805 | 427.363 |
| radical | | | |
| RNA | (—) | 415.876 | 437.573 |
| sugar | (H) | 408.512 | 435.352 |
| radical | | | |
| RNA | (—) | 359.017 | 387.254 |
| base | (H) | 400.563 | 425.971 |
| radical | | | |

^{a}Protonation of the two phosphate groups is denoted as (—) for deprotonated and (H) for protonated state;
^{b}two-layer ONIOM approximation to the G3(MP2)-RAD energies using R(O)MP2/6-311 + G(3df, 2p) method.

^{a} Protonation of the two phosphate groups is denoted as (-) for deprotonated and (H) for protonated state;

### Example 12

### One-Electron Oxidation Products of Model DNA Radicals

Vertical one-electron oxidation (ionization) products of the base and sugar radicals of the deoxyguanosine diphosphate. For the closed-shell zwitterions plotted are their electrostatic potential maps, for the triplet biradicals—spin densities, all in conjunction with the UB3LYP/6-31+G(d) geometries. Also shown are UB3LYP/6-31+G(d) dipole moments (μ, in Debye) and relative energies (Eᵣ, in kJ mol⁻¹) at the UB3LYP/6-31+G(d) and UHF/6-31+G(d) (in brackets) levels

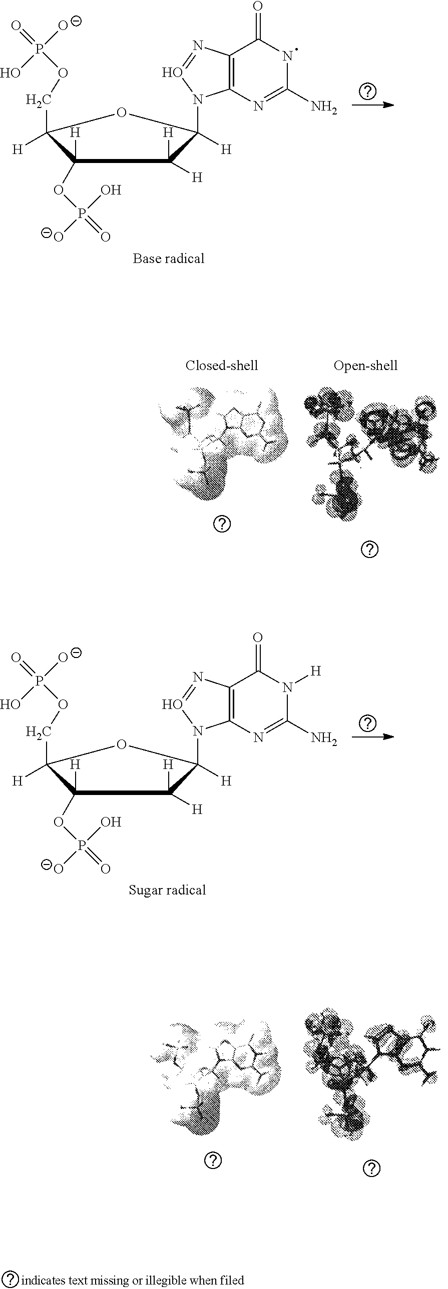

dipole moments (D, Debye) and relative energies (E, kJ mol⁻¹) at the B3LYP/6-31+G(d) and UHF/6-31+G(d) (in brackets) levels.

### Example 13

### NRC Reaction in 100% Tol with CuBr and PMDETA at R.T. (Scheme 1)

100 mg of PMA-Br (Mₙ=4000, 2.5×10⁻⁵ mol), 5.0 mg of 4-carboxylic acid TEMPO (2.5×10⁻⁵ mol) and PMDETA 6.3 μL (3.0×10⁻⁵ mol) were dissolved to 1.0 mL toluene and purged with Ar for 10 min. Then 4.3 mg of CuBr (3.0×10⁻⁵ mol) was added under Ar. The reaction was then sampled at 30 min and stopped the reaction at 20 h. The reaction was diluted by THF and passed through Al₂O₃ to remove the copper. The solution was then analysed by SEC.

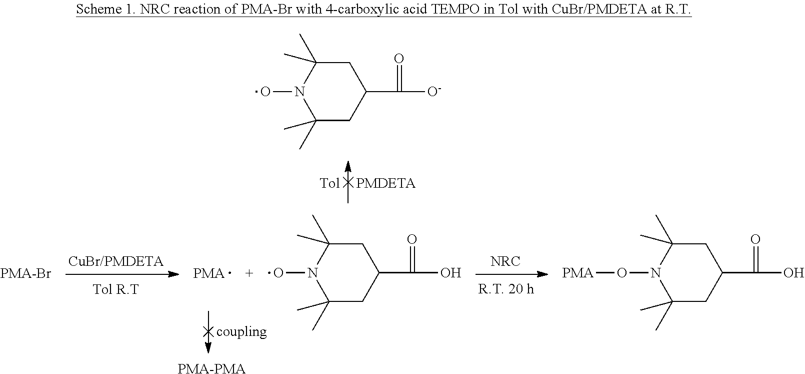

In this reaction the radical has been activated (SOMO=HOMO) and reacts with the PMA radical.

See FIG. 7 for SEC traces of PMA-Br (-), PMA-Br with HOOC-TEMPO reaction in Toluene with CuBr/PMDETA for 30 min (-), and 20 h (-) at R.T.

### Example 14

### NRC Reaction in 100% DMSO with CuBr and Me₆TREN at R.T. (Scheme 2)

85 mg of PMA-Br (Mₙ=4000, 2.125×10⁻⁵ mol), 5.0 mg of 4-carboxylic acid TEMPO (2.5×10⁻⁵ mol) and Me₆TREN 6.3 μL (3.0×10⁻⁵ mol) were dissolved to 1.0 mL DMSO and purged with Ar for 10 min. Then 4.3 mg of CuBr (3.0×10⁻⁵ mol) was added under Ar. The reaction was then sampled at 30 min and stopped the reaction at 20 h. The reaction was diluted by THF and passed through Al₂O₃ to remove the copper. The solution was then analysed by SEC.

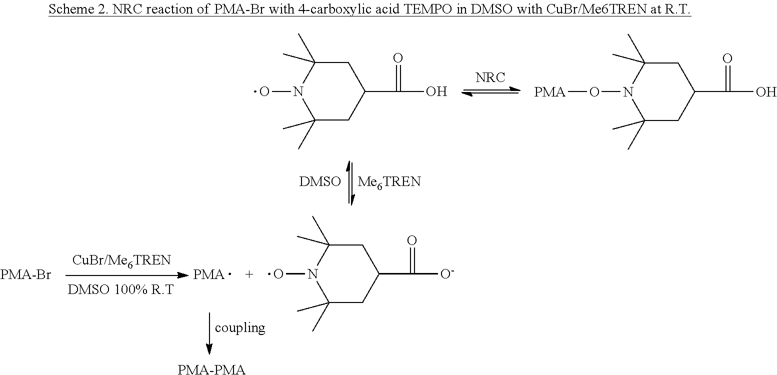

In this reaction the radical has been partially activated. Under these reaction conditions the activated radical is in equilibrium with the (deactivated form, i.e. SOMO≠HOMO) allowing for the PMA radicals to combine.

See FIG. 8 SEC traces of PMA-Br (-), PMA-Br with HOOC-TEMPO reaction in DMSO with CuBr/Me₆TREN for 30 min (-) and 20 h (-) at R.T.

See FIG. 9 Gaussian simulation of PMA-Br with HOOC-TEMPO reaction in DMSO with CuBr/Me₆TREN (-) and LMD simulation (--). It showed 27.3% of coupling product. Simulation: peak 1: Mₚ=4454 PDI=1.05. (PDI used here is lower than the linear PMA-Br 1.06 to make a better fitting). peak 1: Mₚ=8941 PDI=1.02.

### Example 15

### Dissociation Reaction in 100% DMSO with Me₆TREN at R.T. (Scheme 3)

˜50 mg of PMA-ON—COOH (Mₙ=4200) dissolved in 1.0 mL DMSO and purged with Ar for 10 min. Then 50 μL Me₆TREN was added under Ar. The reaction was then sampled at 6 h and stopped the reaction at 20 h. The solution was then analysed by SEC.

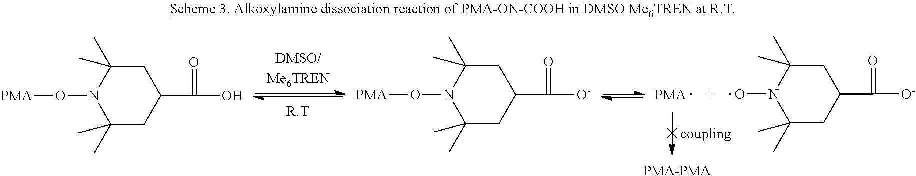

In the above reaction the activated radical (i.e. SOMO=HOMO) coupled to PMA is in equilibrium with the deactivated (i.e. deprotonated, SOMO≠HOMO) radical coupled to PMA. In such an equilibrium, the deactivated (i.e. deprotonated) radical coupled to PMA, dissociates into the stabilised radical and the PMA radical. The PMA radical so deprotected is free to react, in this case with a further PMA radical to give PMA-PMA. In addition the PMA radicals react with each other.

See FIG. 10 SEC traces of purified PMA-ON—COOH kept in THF overnight (-), PMA-ON—COOH in DMSO with Me₆TREN for 6 h (-) and 20 h (-) at R.T.

See FIG. 11 Gaussian simulation of PMA-ON—COOH in DMSO with Me₆TREN for 6 h (-) and LMD simulation (--). It showed 27.2% of coupling product. Simulation: peak 1: Mₚ=4511 PDI=1.05. (PDI used here is lower than the linear PMA-Br 1.06 to make a better fitting). peak 1: Mₚ=9088 PDI=1.02.

See FIG. 12 Gaussian simulation of PMA-ON—COOH in DMSO with Me₆TREN for 20 h (-) and LMD simulation (--). It showed 33.3% of coupling product. Simulation: peak 1: Mₚ=4511 PDI=1.05. (PDI used here is lower than the linear PMA-Br 1.06 to make a better fitting). peak 1: Mₚ=9088 PDI=1.02.

### Tables of Results

TABLE 15
Carboxy-aminoxyl and -peroxyl oxidation products. All values are in
Hartrees.

| | | | | | | |
|---|---|---|---|---|---|---|
| | | UM06- | | | | |
| Species | HLC^{a} | 2X^{b} | MP2^{b} | MP2^{c} | CCSD(T)^{b} | E^{d} |

Carboxy-aminoxyl

| | | | | | | |
|---|---|---|---|---|---|---|
| Closed-shell singlet | −0.37652 | −671.305 | −669.52 | −670.258 | −669.688 | −670.803 |
| (vertical) | | | | | | |
| Closed-shell singlet | −0.37652 | −671.325 | −669.529 | −670.272 | −669.7 | −670.82 |
| (adiabatic) | | | | | | |
| Open-shell triplet | −0.37505 | −671.313 | −669.51 | −670.249 | −669.698 | −670.812 |
| (vertical) | | | | | | |
| Open-shell triplet | −0.37505 | −671.334 | −669.528 | −670.265 | −669.711 | −670.824 |
| (adiabatic) | | | | | | |
| Open-shell singlet (adiabatic) | −671.277 | | | | | |

Carboxy-peroxyl

| | |
|---|---|
| Open-shell triplet (adiabatic) | −495.847 |
| Open-shell singlet (adiabatic) | −495.827 |

^{a}Calculated using G3(MP2)-RAD parameters;
^{c}In conjunction with the 6-31 + G(d) basis set;
^{b}In conjunction with the GTMP2Large basis set;
^{d}G3(MP2,CC)(+) electronic energy.

TABLE 16
Data used for calculating the BDFE-switches in the converted species.
Entropies are in J mol⁻¹ K⁻¹, all other values are in Hartrees.

| Species | T1^{a} | Dispersion^{b} | B3LYP^{c} | M06-2X [symbol] | UHF^{d} | Alpha X^{e} | Beta X^{e} | G3(MP2)-RAD | G3(MP2,CC) | G4(MP2)-6X |
|---|---|---|---|---|---|---|---|---|---|---|
| •CH₃ | 0.007 | −0.00139 | −39.84263 | −39.80797 | −39.57643 | −3.31248 | −2.89324 | −39.78528 | −39.78477 | −39.79360 |
| AA1-R | 0.024 | −0.01657 | −381.73099 | −381.55266 | −379.67805 | −24.26613 | −23.53801 | −381.27734 | −381.27692 | −381.35747 |
| AA1-M | 0.016 | −0.02217 | −421.69116 | −421.49953 | −419.33235 | −27.03610 | −27.03610 | −421.19892 | −421.19892 | −421.28752 |
| AH1-R | 0.019 | −0.01751 | −382.27148 | −382.10090 | −380.24777 | −24.32961 | −23.61997 | −381.83351 | −381.83312 | −381.91367 |
| AH1-M | 0.014 | −0.02294 | −422.24178 | −422.05154 | −419.90349 | −27.11058 | −27.11058 | −421.75888 | −421.75888 | −421.84725 |
| AA2-R | 0.020 | −0.02131 | −421.03806 | −420.84043 | −418.72176 | −27.21014 | −26.49630 | −420.53407 | −420.53367 | −420.62249 |
| AA2-M | 0.015 | −0.02601 | −461.00625 | −460.79255 | −458.38037 | −29.98775 | −29.98775 | −460.46084 | −460.46084 | −460.55757 |
| AH2-R | 0.017 | −0.02225 | −421.58601 | −421.39411 | −419.29412 | −27.28107 | −26.57209 | −421.09546 | −421.09506 | −421.18353 |
| AH2-M | 0.014 | −0.02690 | −461.55964 | −461.34739 | −458.95388 | −30.06276 | −30.06276 | −461.02354 | −461.02354 | −461.11990 |
| AA3-R | 0.019 | −0.02596 | −460.35409 | −460.13491 | −457.76948 | −30.16908 | −29.44303 | −459.79702 | −459.79663 | −459.89479 |
| AA3-M | 0.015 | −0.03160 | −500.31867 | −500.08479 | −497.42392 | −32.94077 | −32.94077 | −499.72145 | −499.72145 | −499.82621 |
| AH3-R | 0.017 | −0.02700 | −460.90121 | −460.68810 | −458.34110 | −30.23497 | −29.52433 | −460.35805 | −460.35765 | −460.45478 |
| AH3-M | 0.013 | −0.03244 | −500.87161 | −500.63898 | −497.99695 | −33.01551 | −33.01551 | −500.28357 | −500.28357 | −500.38799 |
| AA4-R | 0.018 | −0.03070 | −499.66879 | −499.42784 | −496.81547 | −33.11304 | −32.40132 | −499.05860 | −499.05860 | −499.16387 |
| AA4-M | 0.014 | −0.03632 | −539.63291 | −539.37782 | −536.47003 | −35.89223 | −35.89223 | −538.98315 | −538.98315 | −539.09713 |
| AH4-R | 0.016 | −0.03166 | −500.21458 | −499.97959 | −497.38613 | −33.18553 | −32.47585 | −499.61846 | −499.61846 | −499.72382 |
| AH4-M | 0.013 | −0.03725 | −540.18543 | −539.93121 | −537.04222 | −35.96726 | −35.96726 | −539.54474 | −539.54474 | −539.65751 |
| PA0-R | 0.030 | −0.01350 | −417.58423 | −417.40347 | −415.44817 | −25.43637 | −24.61894 | −417.11296 | −417.11251 | −417.20272 |
| PA0-M | 0.017 | −0.01786 | −457.52128 | −457.31960 | −455.08100 | −28.15543 | −28.15543 | −457.00470 | −457.00470 | −457.10144 |
| PH0-R | 0.027 | −0.01408 | −418.12014 | −417.94163 | −416.00864 | −25.49942 | −24.70079 | −417.66012 | −417.65967 | −417.74965 |
| PH0-M | 0.015 | −0.01852 | −458.06645 | −457.86665 | −455.64773 | −28.22924 | −28.22924 | −457.55988 | −457.55988 | −457.65634 |
| PA1-R | 0.028 | −0.01794 | −456.89428 | −456.69262 | −454.49132 | −28.38248 | −27.57348 | −456.37135 | −456.37090 | −456.46953 |
| PA1-M | 0.016 | −0.02250 | −496.83279 | −496.61005 | −494.12472 | −31.10721 | −31.10721 | −496.26395 | −496.26395 | −496.36874 |
| PH1-R | 0.026 | −0.01881 | −457.43612 | −457.23632 | −455.05594 | −28.45235 | −27.65264 | −456.92345 | −456.92300 | −457.02123 |
| PH1-M | 0.015 | −0.02320 | −497.38187 | −497.16071 | −494.69476 | −31.18182 | −31.18182 | −496.82261 | −496.82261 | −496.92707 |
| PA2-R | 0.026 | −0.02268 | −496.20663 | −495.98394 | −493.53648 | −31.33455 | −30.52758 | −495.63148 | −495.63103 | −495.73790 |
| PA2-M | 0.016 | −0.02725 | −536.14653 | −535.90278 | −533.17074 | −34.05965 | −34.05965 | −535.52539 | −535.52539 | −535.63819 |
| PH2-R | 0.025 | −0.02357 | −496.75205 | −496.53101 | −494.10373 | −31.40576 | −30.60528 | −496.18673 | −496.18628 | −496.29275 |
| PH2-M | 0.014 | −0.02799 | −536.69737 | −536.45504 | −533.74215 | −34.13453 | −34.13453 | −536.08556 | −536.08556 | −536.19801 |
| PA3-R | 0.025 | −0.02742 | −535.51906 | −535.27525 | −532.58108 | −34.28587 | −33.48020 | −534.89157 | −534.89112 | −535.00626 |
| PA3-M | 0.015 | −0.03196 | −575.45996 | −575.19505 | −572.21613 | −37.01173 | −37.01173 | −574.78636 | −574.78636 | −574.90718 |
| PH3-R | 0.024 | −0.02832 | −536.06698 | −535.82470 | −533.15038 | −34.35830 | −33.55766 | −535.44909 | −535.44863 | −535.56340 |
| PH3-M | 0.014 | −0.03274 | −576.01213 | −575.74858 | −572.78865 | −37.08686 | −37.08686 | −575.34776 | −575.34776 | −575.46824 |

[symbol]

indicates data missing or illegible when filed

TABLE 17
Acyclic homologues series. All values, except for T1 diagnostics, are in
Hartrees. Homologues are named as follows: first letter denotes alkoxyl (A) and peroxyl
(P) species, second state denotes anionic (A) and protonated (H) carboxylic group,
number corresponds to n in Example 7a, R denotes radical species and M - the
corresponding methyl ethers.

| Species | T1^{a} | Dispersion^{b} | B3LYP^{c} | M06-2X^{c} | UHF^{d} | Alpha X^{e} | Beta X^{e} | G3(MP2)-RAD | G3(MP2,CC) | G4(MP2)-6X |
|---|---|---|---|---|---|---|---|---|---|---|
| •CH3 | 0.007 | −0.00139 | −39.84263 | −39.80797 | −39.57643 | −3.31248 | −2.89324 | −39.78528 | −39.78477 | −39.79360 |
| AA1-R | 0.024 | −0.01657 | −381.73099 | −381.55266 | −379.67805 | −24.26613 | −23.53801 | −381.27734 | −381.27692 | −381.35747 |
| AA1-M | 0.016 | −0.02217 | −421.69116 | −421.49953 | −419.33235 | −27.03610 | −27.03610 | −421.19892 | −421.19892 | −421.28752 |
| AH1-R | 0.019 | −0.01751 | −382.27148 | −382.10090 | −380.24777 | −24.32961 | −23.61997 | −381.83351 | −381.83312 | −381.91367 |
| AH1-M | 0.014 | −0.02294 | −422.24178 | −422.05154 | −419.90349 | −27.11058 | −27.11058 | −421.75888 | −421.75888 | −421.84725 |
| AA2-R | 0.020 | −0.02131 | −421.03806 | −420.84043 | −418.72176 | −27.21014 | −26.49630 | −420.53407 | −420.53367 | −420.62249 |
| AA2-M | 0.015 | −0.02601 | −461.00625 | −460.79255 | −458.38037 | −29.98775 | −29.98775 | −460.46084 | −460.46084 | −460.55757 |
| AH2-R | 0.017 | −0.02225 | −421.58601 | −421.39411 | −419.29412 | −27.28107 | −26.57209 | −421.09546 | −421.09506 | −421.18353 |
| AH2-M | 0.014 | −0.02690 | −461.55964 | −461.34739 | −458.95388 | −30.06276 | −30.06276 | −461.02354 | −461.02354 | −461.11990 |
| AA3-R | 0.019 | −0.02596 | −460.35409 | −460.13491 | −457.76948 | −30.16908 | −29.44303 | −459.79702 | −459.79663 | −459.89479 |
| AA3-M | 0.015 | −0.03160 | −500.31867 | −500.08479 | −497.42392 | −32.94077 | −32.94077 | −499.72145 | −499.72145 | −499.82621 |
| AH3-R | 0.017 | −0.02700 | −460.90121 | −460.68810 | −458.34110 | −30.23497 | −29.52433 | −460.35805 | −460.35765 | −460.45478 |
| AH3-M | 0.013 | −0.03244 | −500.87161 | −500.63898 | −497.99695 | −33.01551 | −33.01551 | −500.28357 | −500.28357 | −500.38799 |
| AA4-R | 0.018 | −0.03070 | −499.66879 | −499.42784 | −496.81547 | −33.11304 | −32.40132 | −499.05860 | −499.05860 | −499.16387 |
| AA4-M | 0.014 | −0.03632 | −539.63291 | −539.37782 | −536.47003 | −35.89223 | −35.89223 | −538.98315 | −538.98315 | −539.09713 |
| AH4-R | 0.016 | −0.03166 | −500.21458 | −499.97959 | −497.38613 | −33.18553 | −32.47585 | −499.61846 | −499.61846 | −499.72382 |
| AH4-M | 0.013 | −0.03725 | −540.18543 | −539.93121 | −537.04222 | −35.96726 | −35.96726 | −539.54474 | −539.54474 | −539.65751 |
| PA0-R | 0.030 | −0.01350 | −417.58423 | −417.40347 | −415.44817 | −25.43637 | −24.61894 | −417.11296 | −417.11251 | −417.20272 |
| PA0-M | 0.017 | −0.01786 | −457.52128 | −457.31960 | −455.08100 | −28.15543 | −28.15543 | −457.00470 | −457.00470 | −457.10144 |
| PH0-R | 0.027 | −0.01408 | −418.12014 | −417.94163 | −416.00864 | −25.49942 | −24.70079 | −417.66012 | −417.65967 | −417.74965 |
| PH0-M | 0.015 | −0.01852 | −458.06645 | −457.86665 | −455.64773 | −28.22924 | −28.22924 | −457.55988 | −457.55988 | −457.65634 |
| PA1-R | 0.028 | −0.01794 | −456.89428 | −456.69262 | −454.49132 | −28.38248 | −27.57348 | −456.37135 | −456.37090 | −456.46953 |
| PA1-M | 0.016 | −0.02250 | −496.83279 | −496.61005 | −494.12472 | −31.10721 | −31.10721 | −496.26395 | −496.26395 | −496.36874 |
| PH1-R | 0.026 | −0.01881 | −457.43612 | −457.23632 | −455.05594 | −28.45235 | −27.65264 | −456.92345 | −456.92300 | −457.02123 |
| PH1-M | 0.015 | −0.02320 | −497.38187 | −497.16071 | −494.69476 | −31.18182 | −31.18182 | −496.82261 | −496.82261 | −496.92707 |
| PA2-R | 0.026 | −0.02268 | −496.20663 | −495.98394 | −493.53648 | −31.33455 | −30.52758 | −495.63148 | −495.63103 | −495.73790 |
| PA2-M | 0.016 | −0.02725 | −536.14653 | −535.90278 | −533.17074 | −34.05965 | −34.05965 | −535.52539 | −535.52539 | −535.63819 |
| PH2-R | 0.025 | −0.02357 | −496.75205 | −496.53101 | −494.10373 | −31.40576 | −30.60528 | −496.18673 | −496.18628 | −496.29275 |
| PH2-M | 0.014 | −0.02799 | −536.69737 | −536.45504 | −533.74215 | −34.13453 | −34.13453 | −536.08556 | −536.08556 | −536.19801 |
| PA3-R | 0.025 | −0.02742 | −535.51906 | −535.27525 | −532.58108 | −34.28587 | −33.48020 | −534.89157 | −534.89112 | −535.00626 |
| PA3-M | 0.015 | −0.03196 | −575.45996 | −575.19505 | −572.21613 | −37.01173 | −37.01173 | −574.78636 | −574.78636 | −574.90718 |
| PH3-R | 0.024 | −0.02832 | −536.06698 | −535.82470 | −533.15038 | −34.35830 | −33.55766 | −535.44909 | −535.44863 | −535.56340 |
| PH3-M | 0.014 | −0.03274 | −576.01213 | −575.74858 | −572.78865 | −37.08686 | −37.08686 | −575.34776 | −575.34776 | −575.46824 |

^{a}T1 diagnostics from CCSD(T)/6-31 + G(d) calculations;
^{b}Dispersion correction to B3LYP, calculated using DFT-D3 procedure with BJ damping;
^{c}Calculated using 6-31 + G(d) basis set;
^{d}Calculated using GTMP2Large basis set;
^{e}Alpha and beta exchange contributions to the HF/CBS energies.

TABLE 18
Cyclic homologues series. All values, except for T1 diagnostics, are in
Hartrees. Homologues are named as follows: first letter denotes alkoxyl (O) and aminoxyl
(N) species, second state denotes anionic (A) and protonated (H) carboxylic group,
number corresponds to n in Example 7a, R denotes radical species and M - the
corresponding methyl ethers.

| Species | T1 a | Dispersion b | B3LYP c | M06-2X c | UHF c | Alpha X e | Beta X e | MP2 d | G3(MP2,CC) | ONIOM f |
|---|---|---|---|---|---|---|---|---|---|---|
| •CH3 | 0.007 | −0.00139 | −39.8426 | −39.808 | −39.561 | −3.31518 | −2.89638 | −39.7313 | −39.7848 | −39.7848 |
| NA0-R | 0.015 | −0.06132 | −671.769 | −671.476 | −667.619 | −45.1612 | −44.3805 | −670.443 | −670.965 | −670.889 |
| NA0-M | 0.015 | −0.07003 | −711.674 | −711.365 | −707.214 | −47.9043 | −47.9043 | −710.26 | −710.832 | −710.755 |
| NH0-R | 0.016 | −0.06238 | −672.31 | −672.018 | −668.174 | −45.2345 | −44.4529 | −670.986 | −671.515 | −671.433 |
| NH0-M | 0.013 | −0.07107 | −712.222 | −711.915 | −707.775 | −47.9784 | −47.9784 | −710.812 | −711.389 | −711.307 |
| NA1-R | 0.015 | −0.06736 | −711.083 | −710.77 | −706.653 | −48.1174 | −47.336 | −709.663 | −710.228 | −710.11 |
| NA1-M | 0.014 | −0.07599 | −750.989 | −750.66 | −746.248 | −50.8599 | −50.8599 | −749.481 | −750.096 | −749.976 |
| NH1-R | 0.015 | −0.06809 | −711.626 | −711.314 | −707.21 | −48.1906 | −47.4088 | −710.208 | −710.78 | −710.655 |
| NH1-M | 0.013 | −0.07675 | −751.538 | −751.21 | −746.81 | −50.9339 | −50.9339 | −750.033 | −750.653 | −750.528 |
| NA2-R | | −0.07176 | −750.392 | −750.057 | −745.683 | −51.0712 | −50.2896 | −748.876 | | −749.322 |
| NA2-M | | −0.08045 | −790.3 | −789.949 | −785.28 | −53.8142 | −53.8142 | −788.696 | | −789.191 |
| NH2-R | | −0.07271 | −750.94 | −750.606 | −746.243 | −51.1452 | −50.3635 | −749.426 | | −749.873 |
| NH2-M | | −0.08141 | −790.852 | −790.503 | −785.844 | −53.8887 | −53.8887 | −789.251 | | −789.746 |
| NA3-R | | −0.07666 | −789.705 | −789.35 | −784.717 | −54.0267 | −53.245 | −788.093 | | −788.54 |
| NA3-M | | −0.08534 | −829.614 | −829.242 | −824.314 | −56.7696 | −56.7696 | −827.914 | | −828.409 |
| NH3-R | | −0.07755 | −790.255 | −789.9 | −785.279 | −54.1004 | −53.3187 | −788.645 | | −789.092 |
| NH3-M | | −0.08624 | −830.166 | −829.796 | −824.879 | −56.8437 | −56.8437 | −828.47 | | −828.965 |
| NA4-R | | −0.08137 | −829.019 | −828.641 | −823.75 | −56.9815 | −56.1998 | −827.311 | | −827.757 |
| NA4-M | | −0.09006 | −868.927 | −868.535 | −863.348 | −59.7246 | −59.7246 | −867.132 | | −867.627 |
| NH4-R | | −0.08229 | −829.569 | −829.193 | −824.313 | −57.0554 | −56.2736 | −827.864 | | −828.311 |
| NH4-M | | −0.09099 | −869.481 | −869.09 | −863.914 | −59.7988 | −59.7988 | −867.689 | | −868.184 |
| NA5-R | | −0.08613 | −868.332 | −867.934 | −862.784 | −59.9366 | −59.1548 | −866.529 | | −866.976 |
| NA5-M | | −0.09482 | −908.242 | −907.828 | −902.383 | −62.6797 | −62.6797 | −906.351 | | −906.846 |
| NH5-R | | −0.08705 | −868.884 | −868.487 | −863.348 | −60.0105 | −59.2287 | −867.084 | | −867.53 |
| NH5-M | | −0.09574 | −908.796 | −908.383 | −902.949 | −62.7538 | −62.7538 | −906.908 | | −907.403 |
| NA4I-R g | | | −829.021 | −828.656 | −823.749 | −56.9908 | −56.2089 | −827.327 | | −827.773 |
| NA4I-M g | | | −868.927 | −868.546 | −863.344 | −59.732 | −59.732 | −867.145 | | −867.64 |
| NH4I-R g | | | −829.567 | −829.196 | −824.31 | −57.0606 | −56.2787 | −827.868 | | −828.315 |
| NH4I-M g | | | −869.478 | −869.092 | −863.911 | −59.8035 | −59.8035 | −867.692 | | −868.187 |
| NAs-R h | | | | −711.943 | | | | | | |
| NAs-M h | | | | −751.837 | | | | | | |
| NHs-R h | | | | −712.498 | | | | | | |
| NHs-M h | | | | −752.394 | | | | | | |
| NA6-R | | −0.09088 | −907.646 | −907.227 | −901.819 | −62.8916 | −62.1097 | −905.747 | | −906.27 |
| NA6-M | | −0.09958 | −947.556 | −947.121 | −941.417 | −65.6347 | −65.6347 | −945.57 | | −946.142 |
| NA7-R | | | | −946.52 | | | | | | |
| NA7-M | | | | −986.414 | | | | | | |
| NA8-R | | | | −985.813 | | | | | | |
| NA8-M | | | | −1025.71 | | | | | | |
| NA9-R | | | | −1025.11 | | | | | | |
| NA9-M | | | | −1065 | | | | | | |
| NA10-R | | | | −1064.4 | | | | | | |
| NA10-M | | | | −1104.29 | | | | | | |
| OA0-R | 0.017 | −0.06428 | −655.72 | −655.429 | −651.637 | −44.6156 | −43.9016 | −654.393 | −654.929 | |
| OA0-M | 0.014 | −0.07255 | −695.681 | −695.375 | −691.272 | −47.3961 | −47.3961 | −694.275 | −694.851 | |
| OH0-R | 0.016 | −0.06527 | −656.266 | −655.977 | −652.197 | −44.6885 | −43.9747 | −654.943 | −655.484 | |
| OH0-M | 0.013 | −0.07355 | −696.232 | −695.927 | −691.835 | −47.4706 | −47.4706 | −694.829 | −695.41 | |
| OA1-R | | −0.07026 | −695.035 | −694.724 | −690.672 | −47.5713 | −46.8573 | −693.614 | | −694.15 |
| OA1-M | | −0.0785 | −734.996 | −734.67 | −730.306 | −50.3517 | −50.3517 | −733.496 | | −734.072 |
| OH1-R | | −0.07124 | −695.58 | −695.271 | −691.231 | −47.6434 | −46.9296 | −694.164 | | −694.705 |
| OH1-M | | −0.07951 | −735.546 | −735.221 | −730.869 | −50.4253 | −50.4253 | −734.049 | | −734.63 |
| OA2-R | | −0.07469 | −734.347 | −734.013 | −729.703 | −50.5252 | −49.8112 | −732.828 | | −733.364 |
| OA2-M | | −0.08295 | −774.307 | −773.96 | −769.338 | −53.306 | −53.306 | −772.711 | | −773.287 |
| OH2-R | | −0.07571 | −734.894 | −734.563 | −730.265 | −50.5981 | −49.8843 | −733.382 | | −733.922 |
| OH2-M | | −0.08399 | −774.859 | −774.512 | −769.902 | −53.3799 | −53.3799 | −773.267 | | −773.848 |
| OA3-R | | −0.07957 | −773.662 | −773.305 | −768.737 | −53.4805 | −52.7666 | −772.046 | | −772.582 |
| OA3-M | | −0.08784 | −813.621 | −813.253 | −808.373 | −56.2614 | −56.2614 | −811.93 | | −812.505 |
| OH3-R | | −0.08051 | −774.208 | −773.856 | −769.3 | −53.5532 | −52.8393 | −772.601 | | −773.141 |
| OH3-M | | −0.0888 | −814.174 | −813.806 | −808.937 | −56.3349 | −56.3349 | −812.486 | | −813.067 |
| OA4-R | | −0.08429 | −812.977 | −812.597 | −807.771 | −56.4353 | −55.7214 | −811.264 | | −811.8 |
| OA4-M | | −0.09256 | −852.935 | −852.545 | −847.407 | −59.2165 | −59.2165 | −851.148 | | −851.724 |
| OH4-R | | −0.0852 | −813.524 | −813.151 | −808.336 | −56.509 | −55.7951 | −811.821 | | −812.361 |
| OH4-M | | −0.09348 | −853.489 | −853.101 | −847.973 | −59.2906 | −59.2906 | −851.706 | | −852.286 |
| OA5-R | | −0.08905 | −852.292 | −851.89 | −846.805 | −59.3904 | −58.6764 | −850.483 | | −851.019 |
| OA5-M | | −0.09732 | −892.25 | −891.838 | −886.441 | −62.1715 | −62.1715 | −890.367 | | −890.942 |
| OH5-R | | −0.08996 | −852.839 | −852.445 | −847.371 | −59.4641 | −58.7502 | −851.04 | | −851.58 |
| OH5-M | | −0.09823 | −892.804 | −892.394 | −887.008 | −62.2457 | −62.2457 | −890.925 | | −891.506 |

a T1 diagnostics from CCSD(T)/6-31 + G(d) calculations;
b Dispersion correction to B3LYP, calculated using DFT-D3 procedure with BJ damping;
c Calculated using 6-31 + G(d) basis set;
d Calculated using 6-311 + G(3df,2p) basis set;
e Alpha and beta exchange contributions to the corresponding HF energies;
f For the aminoxyl series, core layer is the non-substituted TEMPO. for the alkoxyl series core layer is the first homologue of the series;
g Lowest energy conformer of carboxy-TEMPO n = 4 homologue;
h Supermolecule complex of TEMPO and acetic acid, for which the M06-2X energies shown are corrected for the BSSE using counterpoise correction.

TABLE 19
Gas-phase acidities. All values, except for T1 diagnostics, are in Hartrees.

| Species | T1^{a} | TC²⁹⁸ | ZPVE | MP2^{b} | ΔE^{c} | ΔH²⁹⁸ |
|---|---|---|---|---|---|---|
| H⁺ | | | | | 0 | 0.00234 |
| COOH- | 0.016 | 0.01628 | 0.27033 | −670.99094 | −671.51609 | −671.22947 |
| TEMPO• | | | | | | |
| COO⁻-TEMPO• | 0.015 | 0.01611 | 0.25679 | −670.44932 | −670.96611 | −670.69321 |
| COOH- | 0.013 | 0.01772 | 0.30984 | −710.81188 | −711.38915 | −711.06159 |
| TEMPO-CH₃ | | | | | | |
| COO⁻-TEMPO- | 0.015 | 0.01757 | 0.29599 | −710.25982 | −710.83194 | −710.51838 |
| CH₃ | | | | | | |
| COO⁻-TEMPO- | 0.015 | 0.01816 | 0.28934 | −809.40363 | −809.99936 | −809.69185 |
| CH₂F | | | | | | |
| COOH- | 0.014 | 0.0183 | 0.30316 | −809.95268 | −810.55359 | −810.23213 |
| TEMPO-CH₂F | | | | | | |
| COO⁻- | 0.015 | 0.01737 | 0.26054 | −770.18759 | −770.74017 | −770.46225 |
| PROXYL-CH₂F | | | | | | |
| COOH- | 0.014 | 0.01746 | 0.27431 | −770.73266 | −771.29035 | −770.99858 |
| PROXYL-CH₂F | | | | | | |
| COO⁻-PROXYL-CH₂Ph | | 0.02115 | 0.34634 | −901.59348 | −902.1225 | −901.75501 |
| COOH-PROXYL-CH₂Ph | | 0.02123 | 0.36024 | −902.14193 | −902.67602 | −902.29455 |
| COOH- | 0.015 | 0.01565 | 0.24118 | −631.7746 | −632.25478 | −631.99795 |
| PROXYL• | | | | | | |
| COO⁻- | 0.015 | 0.01536 | 0.22799 | −631.23571 | −631.70844 | −631.46509 |
| PROXYL• | | | | | | |
| COOH- | 0.014 | 0.01697 | 0.2808 | −671.58928 | −672.12337 | −671.82561 |
| PROXYL-CH₃ | | | | | | |
| COO⁻- | 0.015 | 0.0167 | 0.26731 | −671.04018 | −671.56919 | −671.28518 |
| PROXYL-CH₃ | | | | | | |
| COO⁻-TEMPO-CH₂Ph | | 0.02221 | 0.37478 | −940.8098 | −941.38192 | −940.98493 |
| COOH-TEMPO-CH₂Ph | | 0.02239 | 0.38858 | −941.36004 | −941.93731 | −941.52633 |
| •CH₃ | 0.007 | 0.00412 | 0.02888 | −39.73174 | −39.78528 | −39.75228 |
| •CH₂F | 0.017 | 0.00396 | 0.02438 | | −138.93365 | −138.90531 |
| •CH₂Ph | 0.02 | 0.00689 | 0.11142 | −270.29202 | −270.34555 | −270.22725 |

^{a}T1 diagnostics from CCSD(T)/6-31 + G(d) calculations;
^{b}Calculated using 6-311 + G(3df,2p) basis set;
^{c}G3(MP2)-RAD(+) electronic energy or an ONIOM approximation to it at the R(O)MP2/6-311 + G(3df,2p) level of theory (given in bold colour).

TABLE 20
Different anionic fragments and nucleic acid radicals. All
values, except for T1 diagnostics, are in Hartrees. Species
are named as follows: in the anionic series first word
corresponds to the anionic fragment, first letter denotes
anionic (A) and protonated (H) states, second letter denotes
radical (R) and corresponding methyl ether (M) species; in
the nucleic acid series G stands for the guanine, d for
deoxyribose and r for ribose moieties, a denotes deprotonated
phosphate groups, 4 refers to C4 sugar radicals and 1-to N1
base radicals, core refers to the ONIOM core species.

| Species | T1^{a} | M06-2X^{b} | R(O)MP2^{c} | G3(MP2, CC)(+) |
|---|---|---|---|---|
| Phosphate A-R | 0.015 | −1165.0885 | −1163.5085 | −1164.1487 |
| Phosphate A-M | 0.014 | −1204.9793 | −1203.321 | −1204.0166 |
| Phosphate H-R | 0.015 | −1165.6055 | −1164.0267 | −1164.6748 |
| Phosphate H-M | 0.013 | −1205.5022 | −1203.8475 | −1204.5485 |
| Sulfate A-R | 0.015 | −1181.9 | −1180.4086 | −1181.0028 |
| Sulfate A-M | 0.014 | −1221.7911 | −1220.2213 | −1220.871 |
| Sulfate H-R | 0.016 | −1182.391 | −1180.9008 | −1181.5023 |
| Sulfate H-M | 0.014 | −1222.2885 | −1220.723 | −1221.3769 |
| Alkoxide A-R | 0.015 | −558.10651 | | −557.68385 |
| Alkoxide A-M | 0.015 | −597.99413 | | −597.54881 |
| Alkoxide H-R | 0.015 | −558.69644 | | −558.28107 |
| Alkoxide H-M | 0.012 | −598.59309 | | −598.15474 |
| Thiocarboxylate | 0.016 | −994.43313 | −993.03519 | −993.57214 |
| A-R | | | | |
| Thiocarboxylate | 0.015 | −1034.3234 | −1032.8467 | −1033.4387 |
| A-M | | | | |
| Thiocarboxylate | 0.016 | −994.95587 | −993.55905 | −994.1028 |
| H-R | | | | |
| Thiocarboxylate | 0.013 | −1034.8528 | −1033.3802 | −1033.976 |
| H-M | | | | |

| | | | | G3(MP2)- | |
|---|---|---|---|---|---|
| Species | T1^{a} | B3LYP^{d} | R(O)MP2^{c} | RAD | E-ONIOM^{e} |
| H• | 0 | −0.50027 | −0.49981 | −0.50171 | −0.50171 |
| G-core | 0.017 | −394.92904 | −394.21193 | −394.46927 | |
| dG | | −2098.9808 | −2095.8735 | | −2096.1308 |
| adG | | −2097.8574 | −2094.7778 | | −2095.0351 |
| G1-core | 0.031 | −394.2686 | −393.54192 | −393.79796 | |
| dG-1 | | −2098.3279 | −2095.2103 | | −2095.4664 |
| adG-1 | | −2097.2212 | −2094.1299 | | −2094.386 |
| rG | | −2174.1928 | −2170.9986 | | −2171.2559 |
| arG | | −2173.0779 | −2169.908 | | −2170.1654 |
| rG-1 | | −2173.5399 | −2170.3359 | | −2170.592 |
| arG-1 | | −2172.4409 | −2169.2601 | | −2169.5161 |
| d-core | 0.01 | −402.33341 | −401.58698 | −401.89499 | |
| dG | | −2098.9808 | −2095.8735 | | −2096.1815 |
| adG | | −2097.8574 | −2094.7778 | | −2095.0858 |
| d4-core | 0.014 | −401.68126 | −400.93187 | −401.23439 | |
| dG4 | | −2098.3267 | −2095.2118 | | −2095.5143 |
| adG4 | | −2097.2003 | −2094.1164 | | −2094.4189 |
| r-core | 0.011 | −477.54304 | −476.70744 | −477.04437 | |
| rG | | −2174.1928 | −2170.9986 | | −2171.3355 |
| arG | | −2173.0779 | −2169.908 | | −2170.2449 |
| r4-core | 0.014 | −476.88366 | −476.04491 | −476.37626 | |
| rG4 | | −2173.5369 | −2170.3366 | | −2170.668 |
| arG4 | | −2172.4193 | −2169.2452 | | −2169.5766 |

^{a}T1 diagnostics from CCSD(T)/6-31 + G(d) calculations;
^{b}Calculated using 6-31 + G(d) basis set;
^{c}Calculated using 6-311 + G(3df, 2p) basis set;
^{d}Calculated using 6-31G(d) basis set;
^{e}ONIOM approximation to G3(MP2)-RAD electronic energy at the R(O)MP2/6-311 + G(3df, 2p) level of theory.

TABLE 21
Switching under different conditions - Absolute X-R BDEs and BDE-
switches, calculated from M06-2X/6-31 + G(d) electronic energies and Gibbs free energies
at 25, 60 and 100° C. in the gas phase and various solvents, all in kJ mol⁻¹

| | | | | | | | Solution | |
|---|---|---|---|---|---|---|---|---|
| | | | Gas phase | | | | ΔG298 b, c | |
| X• | R• | State a | ΔEe | ΔG298 | ΔG333 | ΔG373 | H2O | Toluene |

Absolute BDEs

| | | | | | | | | |
|---|---|---|---|---|---|---|---|---|
| HOOC(CH2)4OO• | •H | (—) | 350.15 | 290.29 | 286.18 | 281.43 | 339.72 | 318.72 |
| | | (H) | 362.64 | 304.00 | 300.04 | 295.46 | 343.18 | 326.78 |
| HOOC(CH2)4OO• | •CH3 | (—) | 291.11 | 219.52 | 213.52 | 206.62 | 245.31 | 238.45 |
| | | (H) | 304.75 | 236.78 | 231.21 | 224.79 | 250.02 | 248.85 |
| HOOC- | •H | (—) | 283.29 | 222.26 | 217.92 | 212.90 | 256.80 | 246.17 |
| TEMPO• | | (H) | 302.57 | 241.77 | 237.49 | 232.55 | 261.66 | 258.77 |
| HOOC- | •CH3 | (—) | 214.16 | 140.24 | 133.91 | 126.61 | 157.12 | 155.70 |
| TEMPO• | | (H) | 233.18 | 159.57 | 153.36 | 146.20 | 161.56 | 167.63 |
| HOOC- | •CH2F | (—) | 259.52 | 182.80 | 175.71 | 167.60 | 188.59 | 190.56 |
| TEMPO• | | (H) | 270.38 | 194.20 | 187.26 | 179.30 | 192.97 | 199.58 |
| HOOC- | •CH2Ph | (—) | 173.19 | 101.65 | 95.03 | 87.46 | 102.13 | 102.92 |
| TEMPO• | | (H) | 187.15 | 115.90 | 109.40 | 101.96 | 105.87 | 112.65 |
| HO3SO- | •CH3 | (—) | 218.14 | 144.52 | 138.26 | 131.05 | 159.35 | 159.27 |
| TEMPO• | | (H) | 235.13 | 161.00 | 154.73 | 147.51 | 163.53 | 169.30 |
| HO(—O2)PO- | •CH3 | (—) | 202.06 | 129.30 | 123.07 | 115.88 | 159.32 | 149.53 |
| TEMPO• | | (H) | 217.24 | 144.33 | 138.13 | 130.99 | 159.58 | 158.54 |
| (HO)2(O)PO- | •CH | (H) | 233.22 | 161.88 | 155.91 | 149.03 | 163.36 | 169.56 |
| TEMPO• | | | | | | | | |
| HOO- | •CH3 | (—) | 213.15 | 139.62 | 133.36 | 126.15 | 157.68 | 154.96 |
| TEMPO• | | (H) | 232.55 | 158.64 | 152.38 | 145.18 | 160.50 | 166.31 |
| HO- | •CH3 | (—) | 209.12 | 135.88 | 129.60 | 122.38 | 155.10 | 151.04 |
| TEMPO• | | (H) | 232.83 | 158.95 | 152.68 | 145.47 | 160.34 | 166.66 |
| HOOC—(CH2)3(CH2═CH)2C• | •CH3 | (—) | 277.65 | 202.02 | 195.76 | 188.55 | 218.90 | 217.44 |
| | | (H) | 284.30 | 210.93 | 204.92 | 197.99 | 221.12 | 222.87 |
| HOOC—(CH2)4(NHCH3)(COOCH3)C• | •CH3 | (—) | 338.57 | 277.98 | 273.81 | 268.96 | 304.15 | 296.36 |
| | | (H) | 346.97 | 285.68 | 281.50 | 276.63 | 304.99 | 299.34 |

BDE-switches (Δ)

| | | | | | | | |
|---|---|---|---|---|---|---|---|
| HOOC(CHCH2)4OO• | •H | 12.49 | 13.71 | 13.86 | 14.04 | 3.46 | 8.06 |
| HOOC(CH2)4OO• | •CH3 | 13.64 | 17.26 | 17.68 | 18.17 | 4.71 | 10.40 |
| HOOC- | •H | 19.27 | 19.51 | 19.57 | 19.65 | 4.86 | 12.60 |
| TEMPO• | | | | | | | |
| HOOC- | •CH3 | 19.02 | 19.33 | 19.45 | 19.59 | 4.43 | 11.92 |
| TEMPO• | | | | | | | |
| HOOC- | •CH2F | 10.86 | 11.41 | 11.54 | 11.70 | 4.38 | 9.02 |
| TEMPO• | | | | | | | |
| HOOC- | •CH2Ph | 13.95 | 14.25 | 14.36 | 14.50 | 3.75 | 9.73 |
| TEMPO• | | | | | | | |
| HO3SO- | •CH3 | 16.99 | 16.48 | 16.47 | 16.46 | 4.17 | 10.03 |
| TEMPO• | | | | | | | |
| HO(—O2)PO- | •CH3 | 15.18 | 15.03 | 15.06 | 15.10 | 0.26 | 9.01 |
| TEMPO• | | | | | | | |
| (HO)2(O)PO- | •CH3 | 15.98 | 17.55 | 17.78 | 18.04 | 3.78 | 11.02 |
| TEMPO* | | | | | | | |
| HOO- | •CH3 | 19.40 | 19.01 | 19.02 | 19.04 | 2.82 | 11.36 |
| TEMPO• | | | | | | | |
| HO- | •CH3 | 23.71 | 23.07 | 23.08 | 23.09 | 5.25 | 15.62 |
| TEMPO• | | | | | | | |
| HOOC—(CH2)3(CH2═CH)2C• | •CH3 | 6.66 | 8.91 | 9.15 | 9.44 | 2.22 | 5.44 |
| HOOC—(CH2)4(NHCH3)(COOCH3)C• | •H | 8.40 | 7.70 | 7.69 | 7.67 | 0.84 | 2.98 |

| | | | Solution ΔG298 b, c | | | |
|---|---|---|---|---|---|---|
| X• | R• | State a | DMSO | ACN | Acetone | DCA |
| Absolute BDEs | | | | | | |
| HOOC(CH2)4OO• | •H | (—) | 327.80 | 323.23 | 326.75 | 326.83 |
| | | (H) | 332.17 | 328.03 | 331.50 | 332.22 |
| HOOC(CH2)4OO• | •CH3 | (—) | 243.64 | 243.76 | 241.46 | 241.63 |
| | | (H) | 249.10 | 249.72 | 247.17 | 248.18 |
| HOOC- | •H | (—) | 253.16 | 252.45 | 253.53 | 251.15 |
| TEMPO• | | (H) | 259.19 | 258.31 | 256.51 | 258.23 |
| HOOC- | •CH3 | (—) | 159.72 | 162.90 | 159.30 | 156.79 |
| TEMPO• | | (H) | 164.95 | 168.34 | 161.73 | 163.61 |
| HOOC- | •CH2F | (—) | 192.02 | 196.12 | 192.44 | 190.47 |
| TEMPO• | | (H) | 196.02 | 200.08 | 193.93 | 195.35 |
| HOOC- | •CH2Ph | (—) | 103.21 | 109.83 | 108.11 | 103.47 |
| TEMPO• | | (H) | 106.92 | 113.41 | 107.76 | 107.55 |
| HO3SO- | •CH3 | (—) | 162.20 | 165.21 | 159.10 | 159.81 |
| TEMPO• | | (H) | 166.92 | 170.35 | 163.70 | 165.03 |
| HO(—O2)PO- | •CH3 | (—) | 161.33 | 161.75 | 157.52 | 157.02 |
| TEMPO• | | (H) | 161.21 | 164.14 | 158.20 | 158.87 |
| (HO)2(O)PO- | •CH | (H) | 166.92 | 170.43 | 164.03 | 165.71 |
| TEMPO• | | | | | | |
| HOO- | •CH3 | (—) | 159.77 | 161.82 | 156.55 | 157.30 |
| TEMPO• | | (H) | 163.84 | 167.15 | 160.87 | 162.59 |
| HO- | •CH3 | (—) | 156.90 | 158.74 | 152.97 | 153.38 |
| TEMPO• | | (H) | 163.65 | 166.96 | 160.51 | 162.39 |
| HOOC—(CH2)3(CH2═CH)2C• | •CH3 | (—) | 220.95 | 222.50 | 218.99 | 218.69 |
| | | (H) | 223.63 | 226.05 | 220.57 | 221.66 |
| HOOC—(CH2)4(NHCH3)(COOCH3)C• | •CH3 | (—) | 298.33 | 297.28 | 296.24 | 297.62 |
| | | (H) | 298.54 | 298.04 | 297.37 | 299.13 |
| BDE-switches (Δ) | | | | | | |
| HOOC(CHCH2)4OO• | •H | | 4.38 | 4.80 | 4.76 | 5.38 |
| HOOC(CH2)4OO• | •CH3 | | 5.46 | 5.96 | 5.71 | 6.55 |
| HOOC- | •H | | 6.03 | 5.87 | 2.98 | 7.08 |
| TEMPO• | | | | | | |
| HOOC- | •CH3 | | 5.23 | 5.44 | 2.43 | 6.82 |
| TEMPO• | | | | | | |
| HOOC- | •CH2F | | 4.00 | 3.96 | 1.49 | 4.88 |
| TEMPO• | | | | | | |
| HOOC- | •CH2Ph | | 3.70 | 3.58 | −0.35 | 4.08 |
| TEMPO• | | | | | | |
| HO3SO- | •CH3 | | 4.72 | 5.14 | 4.59 | 5.22 |
| TEMPO• | | | | | | |
| HO(—O2)PO- | •CH3 | | −0.12 | 2.39 | 0.68 | 1.85 |
| TEMPO• | | | | | | |
| (HO)2(O)PO- | •CH3 | | 5.71 | 6.29 | 5.83 | 6.83 |
| TEMPO* | | | | | | |
| HOO- | •CH3 | | 4.07 | 5.33 | 4.33 | 5.29 |
| TEMPO• | | | | | | |
| HO- | •CH3 | | 6.75 | 8.22 | 7.55 | 9.01 |
| TEMPO• | | | | | | |
| HOOC—(CH2)3(CH2═CH)2C• | •CH3 | | 2.68 | 3.55 | 1.59 | 2.97 |
| HOOC—(CH2)4(NHCH3)(COOCH3)C• | •H | | 0.21 | 0.76 | 1.13 | 1.51 |

a (—) corresponds to deprotonated and (H) to protonated state of the acid-base group;
b Gibbs free energies of solvation are calculated using UAKS-(C)PCM/B3LYP/6-31 + G(d) method;
c ‘DMSO’ stands for dimethylsulfoxide, ‘ACN’—acetonitrile, ‘DCA’—dichloroethane.

### GEOMETRIES OF STUDIED SPECIES

All species had either zero imaginary frequencies, as determined from frequency; level of theory is included in each entry, and correspondingly two entries (M06-2X and BMK) are given for some of the compounds. In that regard, reference is made herewith to the publication in Nature Chemistry (Gryn'ova G., Marshall D., Blanksby, S J, Coote M. L. Nature Chem. (2013), 5, 474-481), which is incorporated herewith by reference, inclusive of the supporting information published in association therewith.

1-94. (canceled)

95. A structure of Formula (I):
RAD-L-NEG (I)
wherein:
RAD is a group comprising a radical;
NEG comprises a negative point charge, which is capable of being neutralised;
L links NEG and RAD;
the radical of RAD is not π-conjugated to the negative point charge of NEG; and
wherein, the lowest Singly-Occupied Molecular Orbital (SOMO) of RAD is lower in energy than in the corresponding structure of Formula (I) when the negative point charge of NEG is neutralized;
wherein the radical is selected from the group consisting of: 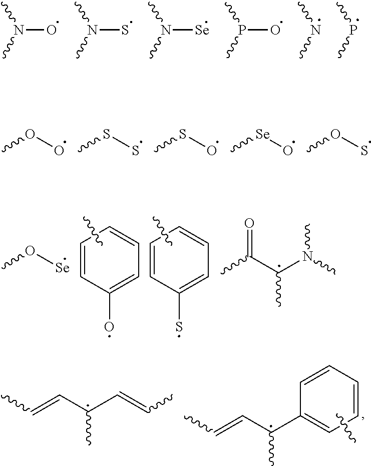
a DNA/RNA-base based radical, or an amino acid based radical; and
wherein the structure of Formula (I) is not:

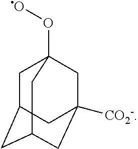

96. The structure of Formula (I) as defined in claim 95, wherein the lowest Singly-Occupied Molecular Orbital (SOMO) of RAD is lower in energy than a Doubly-Occupied Molecular Orbital (DOMO) of NEG; and wherein the SOMO of RAD is higher in energy than the DOMOs of NEG when the negative point charge of NEG is neutralized.

97. The structure of Formula (I) as defined in claim 95, wherein NEG is a surface or structure capable of bearing and/or carrying a negative point charge, and where the negative point charge is capable of being neutralised, wherein the structure capable of bearing and/or carrying a negative point charge is optionally selected from the group comprising: an electrode, a metallic or non-metallic structure, or graphene.

98. The structure of Formula (I) as defined in claim 95, wherein the negative point charge is selected from the group comprising an electron, an electric charge, a negative potential, or a negative coulombic charge; and where the negative point charge is capable of being neutralised by the substantial removal, dissipation or inversion of that charge.

99. The structure of Formula (I) as defined in claim 95, wherein the negative point charge is a group comprising an anion, which is capable of being neutralised by bonding to a proton or other cation, wherein the anion optionally comprises a sterically and/or electronically destabilised anion.

100. The structure of Formula (I) as defined in claim 99, where neutralisation is achieved by:
removal of the proton or other cation bonded to the anion of NEG;
increasing the pH of the reaction medium to remove the proton bonded to the anion of NEG;
addition of a metal chelate to the reaction medium to remove metal cations bonded to the anion of NEG; and/or
addition of anions to the reaction medium, the added anions forming a precipitate with cations present in the reaction medium, thereby removing those cations from bonding with the anions of NEG.

101. A radical protecting group having the structure of Formula (I) as defined in claim 95;
wherein the radical of RAD is capable of forming a bond to a radical to be protected to give the protected radical; and
wherein the radical to be protected is de-protectable when the negative point charge of NEG is not neutralised.

102. A process of deprotecting a radical protected with a radical protecting group as defined in claim 101, by the removal of the cause of neutralisation from a negative point charge that has been neutralised.

103. The process according to claim 102 wherein the deprotected radical reacts with itself, or with one or more reagents in the reaction medium.

104. The process according to claim 103, wherein the radical reaction is selected from the group comprising: radical coupling; Wurtz reaction; nitroxide mediated polymerization; nitroxide radical coupling; double bond addition; cyclization reactions; atom abstraction; and oxidation.

105. The process according to claim 103 wherein the deprotected radical participates in a polymerization reaction.

106. The process according to claim 105, wherein the chain extending polymer radical is capped with the radical protecting group.

107. The process according to claim 106, wherein the capped radical is deprotected, allowing further polymerization of that deprotected radical.

108. The process according to claim 107 wherein the polymer substrate prior to capping is different from the polymer substrate after deprotection.

109. A method of lowering the energy level of the Singly-Occupied Molecular Orbital of RAD and/or raising the energy level of a Doubly-Occupied Molecular Orbital of NEG above the energy level of the Singly-Occupied Molecular Orbital of RAD, by the removal of the cause of neutralisation from a point charge of NEG that has been neutralised, in a structure of Formula (I):
RAD-L-NEG (I)
wherein RAD, NEG, and L are defined in accordance with claim 95.

110. The structure according to claim 95, wherein RAD comprises a steric and/or electronically stabilised radical.

111. The structure according to claim 95, wherein RAD comprises a radical group selected from the group:
2,2′,6,6′-tetramethylpiperidine-N-oxyl (TEMPO),
2,2′,5,5′-tetramethylpyrrolidine-N-oxyl (PROXYL),
2,2,5,5-tetramethyl-4-phenyl-3-azahexane-3-oxyl (TIPNO),
N,N-(1,1-dimethylethyl-1)-(1-diethyl-phosphono-2,2-dimethyl-propyl-1)-N-oxyl (SG1), guanine-based or glycyl-based radical.

112. The structure according to claim 95, wherein NEG comprises an anion selected from the group comprising

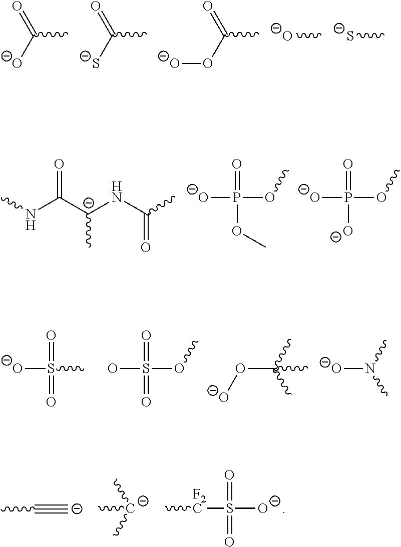

113. The structure according to claim 95, wherein L comprises one or more of: a bond, a hydrogen bond; a non-covalent bond; an electrostatic bond; metal bonding; alkyl, cyclic alkyl, aryl, alkene, alkyne, heterocyclic, heteroaromatic, sugar, metal complex, or is a through-space interaction.

114. The structure according to claim 95, wherein the structure of Formula (I) is selected from the group comprising: 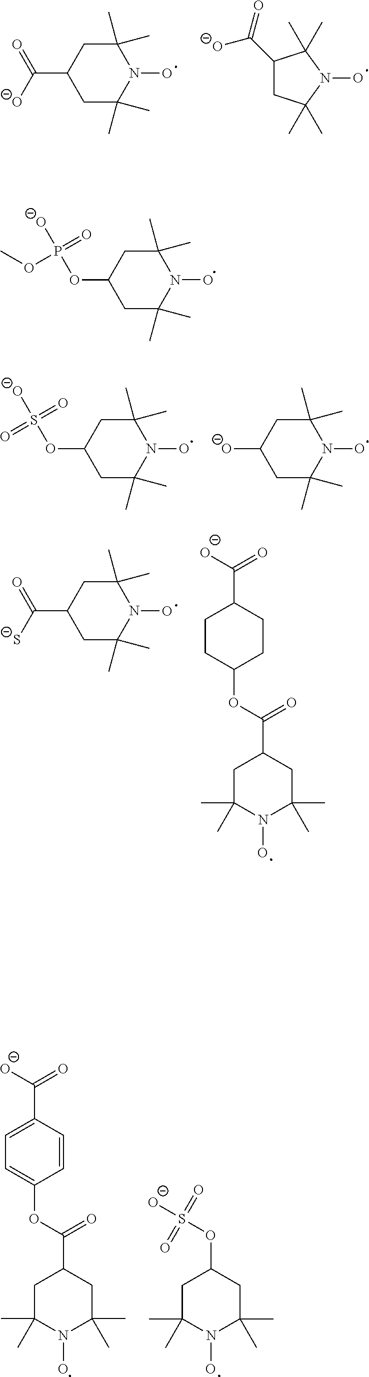 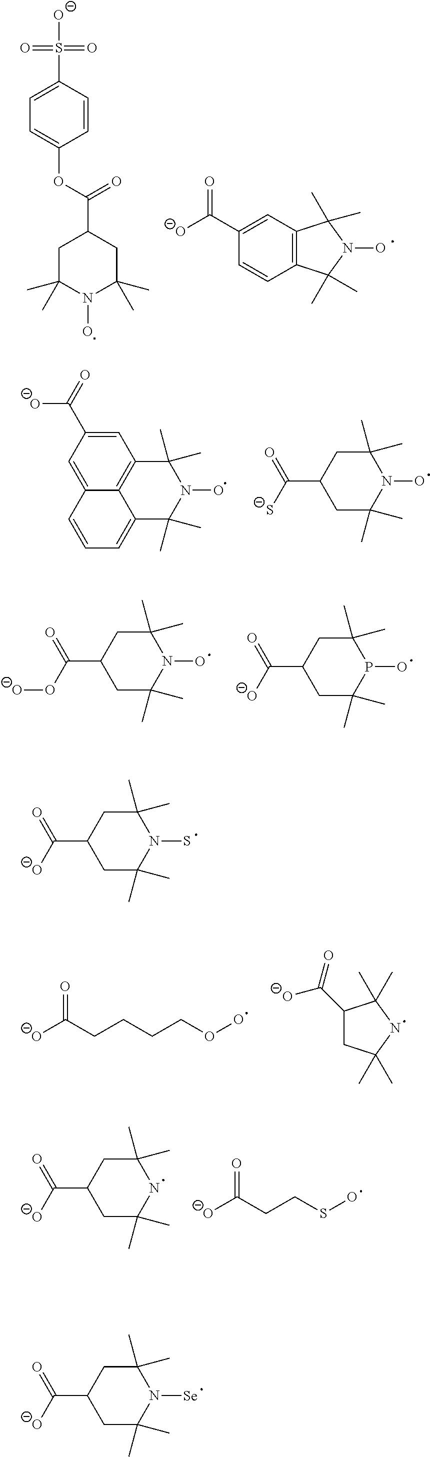 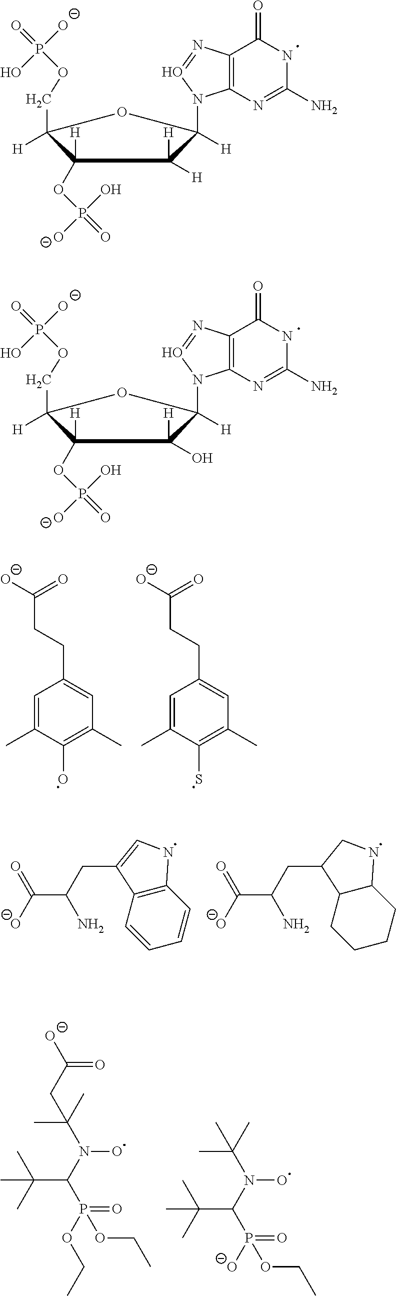 [chemical structure]
